(19) United States
(12) Patent Application Publication
(10) Pub. No.: US 2023/0121608 A1
(43) Pub. Date: Apr. 20, 2023

(54) Compositions and Methods for the Prevention of Stress-Induced Fear, Depressive-Like and Anxiety- Like Behavior

(71) Applicants: The Trustees of Columbia University in the City of New York, New York, NY (US); The Research Foundation for Mental Hygiene, Inc., Menands, NY (US)

(72) Inventors: Christine A. DENNY, Ho-Ho-Kus, NJ (US); Briana K. CHEN, New York, NY (US)

(73) Assignees: The Trustees of Columbia University in the City of New York, New York, NY (US); The Research Foundation for Mental Hygiene, Inc., Menands, NY (US)

(21) Appl. No.: 17/917,339

(22) PCT Filed: Apr. 7, 2021
(86) PCT No.: PCT/US2021/026184

§ 371 Date: Oct. 6, 2022

Related U.S. Application Data
(60) Provisional application No. 63/006,594, filed on Apr. 7, 2020.

(51) Int. Cl. A61K 31/4525; A61K 31/454; A61K 31/445; A61K 31/135; A61P 25/24; A61P 25/22

(52) U.S. Cl. CPC A61K 31/4525; A61K 31/135; A61K 31/445; A61K 31/454; A61P 25/22; A61P 25/24

(57) ABSTRACT

The present disclosure relates to methods and compositions which prevent and protect against all three types of stress-induced maladaptive behaviors- fear, depressive-like, and anxiety-like behavior, which in turn can prevent a wide variety of stress-induced fear, anxiety, and depressive disorders. In some aspects, the compositions and methods use a serotonin 4 receptor (5-hydroxytryptamine (serotonin) receptor 4, or 5-HT₄R) agonist in combination with: ketamine, a ketamine analog, or a pharmaceutically acceptable salt, derivative, or metabolite thereof; an antagonist of the glutamate N-methyl-D-aspartate (NMDA) receptor (NMDAR); or an agonist of the α-amino-3-hydroxy-5-methyl-4-isoxazolepropionic acid (AMPA) receptor (AMPAR). In certain aspects, the present composition or compositions can be administered prior to a stressor. In certain aspects, the present composition or compositions can be administered after a stressor.

### CROSS-REFERENCE TO RELATED APPLICATIONS

The present application claims priority to U.S. Provisional Pat. Application No. 63/006,594 filed on Apr. 7, 2020, which is incorporated herein by reference in its entirety.

### FIELD

The present disclosure relates to methods and compositions which prevent and protect against all three types of stress-induced maladaptive behaviors- fear, depressive-like, and anxiety-like behavior, which in turn can prevent a wide variety of stress-induced fear, anxiety, and depressive disorders. In some aspects, the compositions and methods use a serotonin 4 receptor (5-hydroxytryptamine (serotonin) receptor 4, or 5-HT₄R) agonist in combination with: ketamine, a ketamine analog, or a pharmaceutically acceptable salt, derivative, or metabolite thereof; an antagonist of the glutamate N-methyl-D-aspartate (NMDA) receptor (NMDAR); or an agonist of the α-amino-3-hydroxy-5-methyl-4-isoxazolepropionic acid (AMPA) receptor (AMPAR). In certain aspects, the present composition or compositions can be administered prior to exposure to a stressor. In certain aspects, the present composition or compositions can be administered after exposure to a stressor.

### BACKGROUND

Exposure to stress is a major risk factor for mood and anxiety disorders, including major depressive disorder (MDD) and posttraumatic stress disorder (PTSD). Therapeutic intervention against stress could prevent the onset of mood and anxiety disorders and thereby reduce the associated healthcare burdens. These mental illnesses are a major global health challenge, affecting between 20-30% of adults in the United States alone (NIMH, 2019). The current paradigm for treating these disorders is to mitigate symptoms through medication or therapy after the disorder has already developed. However, this approach can be inefficient, costly, and ineffective in up to one-third of patients (Fava and Davidson, 1996). Enhancing resilience to stress to prevent these disorders before they develop is therefore an innovative approach to significantly reduce the global burden of mood and anxiety disorders.

Previously, the inventors have been shown that a single injection of (R,S)-ketamine prior to a stressor can prevent stress-related depressive-like behavior and attenuate learned fear but does not prevent anxiety-like behavior in male mice (Brachman et al., 2016; McGowan et al., 2017). This result has been replicated in both sexes as well as in rats (Mastrodonato et al., 2018; Amat et al., 2016; Dolzani et al., 2018).

Additionally, the inventors have previously demonstrated that three different serotonin receptor type 4 (5-HT₄R) agonists exhibit prophylactic efficacy (Chen et al., 2020). RS-67,333 attenuates learned fear and prevents anxiety-like behavior in male mice but does not protect against depressive-like behavior. Prucalopride and PF-04995274 attenuate learned fear and prevent depressive-like behavior in male mice but do not alter stress-induced anxiety-like behavior. Moreover, RS-67,333 can protect against anxiety-like behavior in female mice but does not attenuate learned fear or affect depressive-like behavior.

Currently, there are no established drugs given to patients prior to stress exposure to prevent stress-related emotional deficits. The previous disclosures showed that ((R,S)-ketamine, (2R,6R)-HNK, and 5-HT₄R agonists protect against different combinations of fear, depressive-like, and anxiety-like behavior but are not prophylactic against all three types of behaviors in a single drug administration. Thus, there is a need for an improved protocol for preventing all three types of stress related behaviors in both sexes.

### SUMMARY

Disclosed herein are compositions and methods which prevent and protect against all three types of stress-induced maladaptive behaviors- fear, depressive-like, and anxiety-like behavior, making it distinct from earlier compositions and methods which did not protect against all three with a single administration. The current compositions and methods can be used to prevent a wide variety of stress-induced fear, anxiety, and depressive disorders including but not limited to major depressive disorder (MDD) and posttraumatic stress disorder (PTSD).

In addition to reducing the burden of disease, the disclosed compositions and methods can significantly reduce monetary costs associated with treating and managing symptoms of stress-related psychiatric disorders. The current compositions and methods can be used to develop a novel compound with greater and longer-lasting efficacy against stress.

In one aspect, the present disclosure provides for a method for preventing or delaying a stress-induced affective disorder or stress-induced psychopathology in a subject in need thereof. The method comprises administering an effective amount of one or more compositions comprising an activator of serotonin 4 receptor (5-HT₄R) (e.g., an agonist of serotonin 4 receptor (5-HT₄R)), or a pharmaceutically acceptable salt, analog, derivative, or metabolite thereof, and ketamine, a ketamine analog, or a pharmaceutically acceptable salt, derivative, or metabolite thereof, to the subject prior to a stressor.

The present disclosure also provides for a method for inducing and/or enhancing stress resilience in a subject in need thereof. The method comprises administering an effective amount of one or more compositions comprising an activator of serotonin 4 receptor (5-HT₄R) (e.g., an agonist of serotonin 4 receptor (5-HT₄R)), or a pharmaceutically acceptable salt, analog, derivative, or metabolite thereof, and ketamine, a ketamine analog, or a pharmaceutically acceptable salt, derivative, or metabolite thereof, to the subject prior to a stressor.

The present disclosure further provides for a method for preventing or delaying a stress-induced affective disorder or stress-induced psychopathology in a subject in need thereof by administering an effective amount of one or more compositions comprising an activator of serotonin 4 receptor (5-HT₄R) (e.g., an agonist of serotonin 4 receptor (5-HT₄R)), or a pharmaceutically acceptable salt, analog, derivative, or metabolite thereof, and an antagonist of the glutamate N-methyl-D-aspartate (NMDA) receptor (NMDAR), to the subject prior to a stressor.

The present disclosure also provides for a method for inducing and/or enhancing stress resilience in a subject in need thereof. The method may comprise administering an effective amount of one or more compositions comprising an activator of serotonin 4 receptor (5-HT₄R) (e.g., an agonist of serotonin 4 receptor (5-HT₄R)), or a pharmaceutically acceptable salt, analog, derivative, or metabolite thereof, and an antagonist of the glutamate N-methyl-D-aspartate (NMDA) receptor (NMDAR), to the subject prior to a stressor.

Additionally, the present disclosure provides for a method for preventing or delaying a stress-induced affective disorder or stress-induced psychopathology in a subject in need thereof by administering an effective amount of one or more compositions comprising an activator of serotonin 4 receptor (5-HT₄R) (e.g., an agonist of serotonin 4 receptor (5-HT₄R)), or a pharmaceutically acceptable salt, analog, derivative, or metabolite thereof, and an agonist of the α-amino-3-hydroxy-5-methyl-4-isoxazolepropionic acid (AMPA) receptor (AMPAR), to the subject prior to a stressor.

The present disclosure also provides for a method for inducing and/or enhancing stress resilience in a subject in need thereof. The method may comprise administering an effective amount of one or more compositions comprising an activator of serotonin 4 receptor (5-HT₄R) (e.g., an agonist of serotonin 4 receptor (5-HT₄R)), or a pharmaceutically acceptable salt, analog, derivative, or metabolite thereof, and an agonist of the α-amino-3-hydroxy-5-methyl-4-isoxazolepropionic acid (AMPA) receptor (AMPAR), to the subject prior to a stressor.

In the preceding embodiments, the administration of the activator or agonist of 5-HT₄R in combination with either ketamine, a ketamine analog, or a pharmaceutically acceptable salt, derivative, or metabolite thereof, a NMDAR antagonist, or an AMPAR agonist prevents all three types of stress-induced maladaptive behaviors- fear, depressive-like, and anxiety-like behavior,

Thus, the present disclosure also provides for a method of preventing, delaying or diminishing three types of stress-induced maladaptive behaviors, fear, depressive-like, and anxiety-like behavior. The method comprises administering an effective amount of one or more compositions comprising an activator of serotonin 4 receptor (5-HT₄R) (e.g., an agonist of serotonin 4 receptor (5-HT₄R)), or a pharmaceutically acceptable salt, analog, derivative, or metabolite thereof, and ketamine, a ketamine analog, or a pharmaceutically acceptable salt, derivative, or metabolite thereof, to the subject prior to a stressor.

The present disclosure further provides for a method of preventing, delaying or diminishing three types of stress-induced maladaptive behaviors, fear, depressive-like, and anxiety-like behavior comprising the administration of an effective amount of one or more compositions comprising an activator of serotonin 4 receptor (5-HT₄R) (e.g., an agonist of serotonin 4 receptor (5-HT₄R)), or a pharmaceutically acceptable salt, analog, derivative, or metabolite thereof, and an antagonist of the glutamate N-methyl-D-aspartate (NMDA) receptor (NMDAR), to the subject prior to a stressor.

The present disclosure also provides for a method of preventing, delaying or diminishing three types of stress-induced maladaptive behaviors, fear, depressive-like, and anxiety-like behavior comprising the administration of an effective amount of one or more compositions comprising an activator of serotonin 4 receptor (5-HT₄R) (e.g., an agonist of serotonin 4 receptor (5-HT₄R)), or a pharmaceutically acceptable salt, analog, derivative, or metabolite thereof, and an agonist of the α-amino-3-hydroxy-5-methyl-4-isoxazolepropionic acid (AMPA) receptor (AMPAR), to the subject prior to a stressor.

In a further aspect, the present disclosure provides for a method for preventing or delaying a stress-induced affective disorder or stress-induced psychopathology in a subject in need thereof. The method comprises administering an effective amount of one or more compositions comprising an activator of serotonin 4 receptor (5-HT₄R) (e.g., an agonist of serotonin 4 receptor (5-HT₄R)), or a pharmaceutically acceptable salt, analog, derivative, or metabolite thereof, and ketamine, a ketamine analog, or a pharmaceutically acceptable salt, derivative, or metabolite thereof, to the subject after exposure to a stressor.

The present disclosure also provides for a method for inducing and/or enhancing stress resilience in a subject in need thereof. The method comprises administering an effective amount of one or more compositions comprising an activator of serotonin 4 receptor (5-HT₄R) (e.g., an agonist of serotonin 4 receptor (5-HT₄R)), or a pharmaceutically acceptable salt, analog, derivative, or metabolite thereof, and ketamine, a ketamine analog, or a pharmaceutically acceptable salt, derivative, or metabolite thereof, to the subject after exposure to a stressor.

The present disclosure further provides for a method for preventing or delaying a stress-induced affective disorder or stress-induced psychopathology in a subject in need thereof by administering an effective amount of one or more compositions comprising an activator of serotonin 4 receptor (5-HT₄R) (e.g., an agonist of serotonin 4 receptor (5-HT₄R)), or a pharmaceutically acceptable salt, analog, derivative, or metabolite thereof, and an antagonist of the glutamate N-methyl-D-aspartate (NMDA) receptor (NMDAR), to the subject after exposure to a stressor.

The present disclosure also provides for a method for inducing and/or enhancing stress resilience in a subject in need thereof. The method may comprise administering an effective amount of one or more compositions comprising an activator of serotonin 4 receptor (5-HT₄R) (e.g., an agonist of serotonin 4 receptor (5-HT₄R)), or a pharmaceutically acceptable salt, analog, derivative, or metabolite thereof, and an antagonist of the glutamate N-methyl-D-aspartate (NMDA) receptor (NMDAR), to the subject after exposure to a stressor.

Additionally, the present disclosure provides for a method for preventing or delaying a stress-induced affective disorder or stress-induced psychopathology in a subject in need thereof by administering an effective amount of one or more compositions comprising an activator of serotonin 4 receptor (5-HT₄R) (e.g., an agonist of serotonin 4 receptor (5-HT₄R)), or a pharmaceutically acceptable salt, analog, derivative, or metabolite thereof, and an agonist of the α-amino-3-hydroxy-5-methyl-4-isoxazolepropionic acid (AMPA) receptor (AMPAR), to the subject after exposure to a stressor.

The present disclosure also provides for a method for inducing and/or enhancing stress resilience in a subject in need thereof. The method may comprise administering an effective amount of one or more compositions comprising an activator of serotonin 4 receptor (5-HT₄R) (e.g., an agonist of serotonin 4 receptor (5-HT₄R)), or a pharmaceutically acceptable salt, analog, derivative, or metabolite thereof, and an agonist of the α-amino-3-hydroxy-5-methyl-4-isoxazolepropionic acid (AMPA) receptor (AMPAR), to the subject after exposure to a stressor.

In a further aspect, the disclosure provides for a method of preventing, delaying or diminishing three types of stress-induced maladaptive behaviors, fear, depressive-like, and anxiety-like behavior comprising administering an effective amount of one or more compositions comprising an activator of serotonin 4 receptor (5-HT₄R) (e.g., an agonist of serotonin 4 receptor (5-HT₄R)), or a pharmaceutically acceptable salt, analog, derivative, or metabolite thereof, and ketamine, a ketamine analog, or a pharmaceutically acceptable salt, derivative, or metabolite thereof, to the subject after exposure to a stressor.

The present disclosure further provides for a method of preventing, delaying or diminishing three types of stress-induced maladaptive behaviors, fear, depressive-like, and anxiety-like behavior comprising the administration of an effective amount of one or more compositions comprising an activator of serotonin 4 receptor (5-HT₄R) (e.g., an agonist of serotonin 4 receptor (5-HT₄R)), or a pharmaceutically acceptable salt, analog, derivative, or metabolite thereof, and an antagonist of the glutamate N-methyl-D-aspartate (NMDA) receptor (NMDAR), to the subject after exposure to a stressor.

The present disclosure also provides for a method of preventing, delaying or diminishing three types of stress-induced maladaptive behaviors, fear, depressive-like, and anxiety-like behavior comprising the administration of an effective amount of one or more compositions comprising an activator of serotonin 4 receptor (5-HT₄R) (e.g., an agonist of serotonin 4 receptor (5-HT₄R)), or a pharmaceutically acceptable salt, analog, derivative, or metabolite thereof, and an agonist of the α-amino-3-hydroxy-5-methyl-4-isoxazolepropionic acid (AMPA) receptor (AMPAR), to the subject after exposure to a stressor.

In some embodiments, the activator or agonist of HT₄R and the ketamine, a ketamine analog, or a pharmaceutically acceptable salt, derivative, or metabolite thereof, the NMDAR antagonist, or the AMPAR agonist are in the same composition. In some embodiments, the agonist or activator of HT₄R and the ketamine, a ketamine analog, or a pharmaceutically acceptable salt, derivative, or metabolite thereof, the NMDAR antagonist, or the AMPAR agonist are in different compositions. In these embodiments, the compositions can be administered simultaneously or sequentially.

The activator or agonist of 5-HT₄R includes but is not limited to 1-(4-amino-5-chloro-2-methoxyphenyl)-3-[1(n-butyl)-4-piperidinyl]-1-propanone HCl (RS-67,333 or RS67333), 4-amino-5-chloro-2,3-dihydro-N-[1-3-methoxypropyl)-4-piperidinyl]-7-benzofuran carboxamide monohydrochloride (prucalopride), 4-[4-[4-tetrahydrofuran-3-yloxy)-benzo[d]isoxazol-3-yloxymethyl]-piperidin-1-ylmethyl]-tetrahydropyran-4-ol(PF-04995274), or combinations thereof.

In some embodiments, the disclosure provides for a method for preventing or delaying a stress-induced affective disorder or stress-induced psychopathology in a subject in need thereof by administering an effective amount of one or more compositions comprising prucalopride or a pharmaceutically acceptable salt, analog, derivative, or metabolite thereof, and ketamine, a ketamine analog, or a pharmaceutically acceptable salt, derivative, or metabolite thereof, to the subject either prior to, or after exposure to, a stressor.

The present disclosure also provides for a method for inducing and/or enhancing stress resilience in a subject in need thereof by administering an effective amount of one or more compositions comprising prucalopride or a pharmaceutically acceptable salt, analog, derivative, or metabolite thereof, and ketamine, a ketamine analog, or a pharmaceutically acceptable salt, derivative, or metabolite thereof, to the subject either prior to, or after exposure to, a stressor.

The present disclosure also provides for a method of preventing, delaying or diminishing three types of stress-induced maladaptive behaviors, fear, depressive-like, and anxiety-like behavior. The method comprises administering an effective amount of one or more compositions comprising prucalopride, or a pharmaceutically acceptable salt, analog, derivative, or metabolite thereof, and ketamine, a ketamine analog, or a pharmaceutically acceptable salt, derivative, or metabolite thereof, to the subject prior to, or after exposure to, a stressor.

In some embodiments, the prucalopride and the ketamine, a ketamine analog, or a pharmaceutically acceptable salt, derivative, or metabolite thereof are in the same composition. In some embodiments, the prucalopride and the ketamine, a ketamine analog, or a pharmaceutically acceptable salt, derivative, or metabolite thereof are in different compositions. In these embodiments, the compositions can be administered simultaneously or sequentially.

The composition or compositions are administered to the subject about 48 hours to about 3 weeks prior to a stressor. In certain embodiments, the composition or compositions are administered to the subject about 72 hours to about 2 weeks prior to a stressor. In certain embodiments, the composition or compositions are administered to the subject about 1 week prior to a stressor.

In certain embodiments, the composition or compositions are administered to the subject once prior to a stressor.

The composition or compositions are administered to the subject about 1 hour to about 1 week after exposure to a stressor. In certain embodiments, the composition or compositions are administered to the subject about 12 hours to about 2 weeks after exposure to a stressor. In certain embodiments, the composition or compositions are administered to the subject about 1 day after exposure to a stressor.

In certain embodiments, the composition or compositions are administered to the subject once after exposure to a stressor.

In certain embodiments, the composition or compositions are administered in a booster series.

In certain embodiments, the composition or compositions are administered at least once prior to the stressor and at least once after the stressor.

In certain embodiments, the composition or compositions are administered orally, intravenously, intranasally, or via injection to the subject.

The stress-induced affective disorders that can be prevented or delayed by the disclosed compositions and methods include but are not limited to major depressive disorder (MDD) and/or posttraumatic stress disorder (PTSD). In certain embodiments, the stress-induced affective disorder is selected from the group consisting of: depressive-like behavior and associated affective disorders; anhedonic behavior and associated affective disorders; anxiety and associated affective disorders; cognitive impairments and deficits and associated disorders; stress-induced fear; and combinations thereof.

In additional embodiments, the stress-induced affective disorder comprises stress-induced psychopathology. In certain embodiments, the stress-induced psychopathology comprises depressive and/or anxious behavior.

The present method may further comprise administering to the subject an effective amount of an anti-depressant, an anxiolytic, or combinations thereof.

The present method may further comprise administering an effective amount of a selective serotonin reuptake inhibitor (SSRI), or a pharmaceutically acceptable salt or derivative thereof.

The present method may further comprise administering an effective amount of fluoxetine, paroxetine, sertraline, lithium, riluzole, prazosin, lamotrigine, ifenprodil, or combinations thereof.

The subject may be a mammal. In certain embodiments, the subject is a human. The subject may be female or male.

### BRIEF DESCRIPTION OF THE FIGURES

For the purpose of illustrating the invention, there are depicted in drawings certain embodiments of the invention. However, the invention is not limited to the precise arrangements and instrumentalities of the embodiments depicted in the drawings.

FIG. 1. Combined (R,S)-ketamine and prucalopride protects against stress in male 129S6/SvEv mice. FIG. 1A shows the experimental design. Saline, (R,S)-ketamine, prucalopride, or a combined dose of prucalopride and (R,S)-ketamine at different dosage combinations was administered 1 week prior to CFC stress in male 129S6/SvEv mice. Five days later, mice were re-exposed to the training context and then assayed for fear, behavioral despair, and perseverative behavior. FIG. 1B is a graph showing that the average freezing was comparable across all groups during CFC training. FIG. 1C shows average freezing upon re-exposure in all groups of mice. Mice administered (R,S)-ketamine (30 mg/kg), prucalopride (3 mg/kg), and (R,S)-ketamine + prucalopride (10 + 3 mg/kg) exhibited reduced freezing compared to saline controls. FIG. 1D shows immobility time on day 1 of the FST and that all groups exhibited comparable immobility time. FIG. 1E shows immobility time on day 2 of the FST and that (R,S)-ketamine (30 mg/kg), prucalopride (1.5 and 3 mg/kg), and (R,S)-ketamine + prucalopride (10 + 3, 10 + 10, and 30 + 10 mg/kg) decreased immobility time when compared to saline-administered mice. FIG. 1F is a graph of the distance traveled in the OF. All groups of mice traveled a comparable distance. FIG. 1G is a graph showing the amount of time in the center of the EPM. All groups of mice spent a comparable amount of time in the center of the EPM. FIG. 1H is a graph showing the amount of time in the open arms of the EPM. All groups of mice spent a comparable amount of time in the open arms of the EPM. FIG. 1I is a graph showing the numbers of entries into the open arms of the EPM. (R,S)-ketamine + prucalopride (10 + 1.5 mg/kg) increased the number of entries into the open arms of the EPM compared to saline. FIG. 1J is a graph of the fraction of mice not feeding versus latency to feed in OF in seconds. FIG. 1K is a graph of the latency to feed in OF in seconds in all of the groups of mice. FIGS. 1J and 1K show that (R,S)- ketamine + prucalopride (10 + 3 mg/kg) protected against stress-induced hyponeophagia in the NSF paradigm. FIG. 1L is a graph showing latency to feed in HC in seconds in all of the groups of mice. FIG. 1M is a graph of body weight loss in each group of mice. FIGS. 1L and 1M show that none of the drugs administered altered latency to feed in the home cage or body weight loss in the NSF. (n = 5-15 male mice per group). Error bars represent + SEM. * p < 0.05. ** p < 0.01. *** p < 0.0001. Sal, saline; K, (R,S)-ketamine; P, prucalopride; CFC, contextual fear conditioning; FST, forced swim test; OF, open field; EPM, elevated plus maze; NSF, novelty suppressed feeding; HC, home cage; cm, centimeter; no, number; sec, second; mg, milligram; kg, kilogram; g, gram.

FIG. 2. Combined (R,S)-ketamine and prucalopride protects against stress in female 129S6/SvEv mice. FIG. 2A shows the experimental design. Saline, (R,S)-ketamine, prucalopride, or a combined dose of prucalopride and (R,S)-ketamine at different dosage combinations was administered 1 week prior to CFC stress in female 129S6/SvEv mice. Five days later, mice were re-exposed to the training context and then assayed for fear, behavioral despair, and perseverative behavior. FIG. 2B is a graph showing that the average freezing was comparable across all groups during CFC training. FIG. 2C shows average freezing upon re-exposure in all groups of mice was comparable. FIG. 2D shows immobility time on day 1 of the FST. FIG. 2E shows the immobility time on day 2 of the FST. FIGS. 2D and 2E show that on days 1 and 2 of the FST, (R,S)-ketamine (10 mg/kg), prucalopride (1.5 mg/kg), and (R,S)-ketamine + prucalopride (10 + 1.5 mg/kg) significantly reduced immobility time compared to saline controls. FIG. 2F is a graph of the distance traveled in the OF. No drugs assayed altered behavior in the OF. FIG. 2G is a graph showing the amount of time in the center of the EPM. All groups of mice spent a comparable amount of time in the center of the EPM. FIG. 2H is a graph showing the amount of time in the open arms of the EPM. FIG. 2I is a graph of entry into the open arms of the EPM. FIGS. 2H and 2I show prucalopride (3 mg/kg) increased time in the open arms and entries into the open arms of the EPM when compared with saline controls. FIG. 2J is a graph of the fraction of mice not feeding versus latency to feed in OF in seconds. FIG. 2K is a graph of the latency to feed in OF in seconds of all of the groups of mice. FIGS. 2J and 2K show that (R,S)-ketamine + prucalopride (10 + 1.5 mg/kg) prevented stress-induced hyponeophagia in the NSF paradigm. (n = 4-10 female mice per group). FIG. 2L is a graph showing latency to feed in HC in seconds in all groups of mice. FIG. 2M is a graph of body weight loss in each group of mice. FIGS. 2L and 2M show that none of the drugs administered altered latency to feed in the home cage or body weight loss in the NSF. (n = 6-12 female mice per group). Error bars represent ± SEM. * p < 0.05. ** p < 0.01. *** p < 0.0001. Sal, saline; K, (R,S)-ketamine; P, prucalopride; CFC, contextual fear conditioning; FST, forced swim test; OF, open field; EPM, elevated plus maze; NSF, novelty suppressed feeding; HC, home cage; cm, centimeter; no, number; sec, second; mg, milligram; kg, kilogram; g, gram.

FIG. 3. Combined (R,S)-ketamine and prucalopride attenuates learned fear and reduces behavioral despair when administered directly after stress in male 129S6/SvEv mice. FIG. 3A shows the behavioral paradigm. Saline, (R,S)-ketamine (30 mg/kg), prucalopride (3 mg/kg), or a combined dose of (R,S)-ketamine and prucalopride (10 + 3 mg/kg) was administered 5 minutes after CFC stress in male 129S6/SvEv mice. FIG. 3B is a graph showing freezing was comparable in all mice during CFC training. FIG. 3C is a bar graph showing freezing during CFC re-exposure. FIG. 3C shows that combined (R,S)-ketamine + prucalopride (10 + 3 mg/kg) significantly reduced freezing during CFC re-exposure. FIG. 3D is a graph of immobility in FST day 1. Behavior was comparable across all groups during FST day 1. FIG. 3E is a graph of immobility in FST day 2. FIG. 3E shows that (R,S)-ketamine (30 mg/kg) and combined (R,S)-ketamine + prucalopride (10 + 3 mg/kg) significantly reduced behavioral despair when compared with saline controls. FIG. 3F is a graph of the total distance traveled in the OF. FIG. 3G is a graph showing the amount of time in the center of the EPM. FIG. 3H is a graph showing the distance traveled in the center of the EPM. FIG. 3I is a graph showing the amount of time in the open arms and center of the EPM. FIG. 3J is a graph showing the numbers of entries into the open arms and center of the EPM. FIG. 3K is a graph showing the amount of time in the closed arms of the EPM. FIG. 3L is a graph showing the numbers of entries into the closed arms of the EPM. FIG. 3M is a graph of the fraction of mice not feeding versus latency to feed in OF in seconds. FIG. 3N is a graph of the latency to feed in OF in seconds of all of the groups of mice. FIG. 3O is a graph showing latency to feed in HC in seconds of all of the groups of mice. FIG. 3P is a graph of body weight loss in each group of mice. FIGS. 3F - 3P show that behavior was comparable across all groups in the OF, EPM, and NSF. Error bars represent ± SEM. * p < 0.05. ** p < 0.01. CFC, contextual fear conditioning; Sal, saline; K, (R,S)-ketamine; P, prucalopride; min, minute; FST, forced swim test; OF, open field; EPM, elevated plus maze; NSF, novelty suppressed feeding; mg, milligram; kg, kilogram; sec, second; cm, centimeter; m, meter; no, number; HC, home cage; g, gram.

FIG. 4. Combined (R,S)-ketamine and prucalopride attenuates learned fear and reduces behavioral despair when administered directly after stress in female 129S6/SvEv mice. FIG. 4A shows the behavioral paradigm. Saline, (R,S)-ketamine (10 mg/kg), prucalopride (1.5 mg/kg), or a combined dose of (R,S)-ketamine and prucalopride (10 + 1.5 mg/kg) was administered 5 minutes after CFC stress in female 129S6/SvEv mice. FIG. 4B is a graph showing freezing was comparable in all mice during CFC training. FIG. 4C is a graph showing freezing during CFC re-exposure and that freezing was comparable in all mice during CFC re-exposure. FIG. 4D is a graph of immobility in FST day 1. FIG. 4D shows combined (R,S)-ketamine and prucalopride (10 + 1.5 mg/kg) significantly reduced immobility time on day 1 of the FST. FIG. 4E is a graph of immobility in FST day 2. FIG. 4E shows that (R,S)-ketamine (10 mg/kg) and combined (R,S)-ketamine + prucalopride (10 + 1.5 mg/kg) significantly reduced behavioral despair when compared with saline controls. FIG. 4F is a graph of the distance traveled in the OF. FIG. 4G is a graph showing the amount of time in the center of the EPM. FIG. 4H is a graph showing the distance traveled in the center of the EPM. FIG. 4I is a graph showing the amount of time in the open arms and center of the EPM. FIG. 4J is a graph showing the numbers of entries into the open arms and center of the EPM. FIG. 4K is a graph showing the amount of time in the closed arms of the EPM. FIG. 4L is a graph showing the numbers of entries into the closed arms of the EPM. FIGS. 4F - 4L show that behavior was comparable across all groups in the OF and EPM. FIG. 4M is a graph of the fraction of mice not feeding versus latency to feed in OF in seconds. FIG. 4N is a graph of the latency to feed in OF in seconds in all of the groups of mice. FIGS. 4M and 4N show that combined (R,S)-ketamine and prucalopride (10 + 1.5 mg/kg) suppressed hyponeophagia in the NSF. FIG. 4O is a graph showing latency to feed in HC in seconds in all of the groups of mice. FIG. 4P is a graph of body weight loss in each group of mice. Error bars represent ± SEM. * p < 0.05. ** p < 0.01. CFC, contextual fear conditioning; Sal, saline; K, (R,S)-ketamine; P, prucalopride; min, minute; FST, forced swim test; OF, open field; EPM, elevated plus maze; NSF, novelty suppressed feeding; mg, milligram; kg, kilogram; sec, second; cm, centimeter; m, meter; no, number; HC, home cage; g, gram.

FIG. 5. Prophylactic administration of combined (R,S)-ketamine and prucalopride suppresses hyponeophagia in female C57BL/6 mice. FIG. 5A shows the behavioral paradigm. Saline, (R,S)-ketamine (10 mg/kg), or (R,S)-ketamine + prucalopride (10 + 1.5 mg/kg) was administered one week before CFC stress. FIG. 5B is a graph showing freezing was comparable in all mice during CFC training. FIG. 5C is a graph showing freezing during CFC re-exposure. FIG. 5C shows freezing levels were significantly increased in mice administered (R,S)-ketamine (10 mg/kg) or (R,S)-ketamine and prucalopride (10 + 1.5 mg/kg) when compared with saline-administered mice. FIG. 5D is a graph of immobility in FST day 1. FIG. 5E is a graph of immobility in FST day 2. No drugs tested altered immobility time in the FST. FIG. 5F is a line graph of the distance traveled in the OF. FIG. 5G is a graph showing the amount of time in the center of the EPM. FIG. 5H is a graph showing the time in the open arms and center of the EPM. FIG. 5I is a graph showing the numbers of entries into the open arms and center of the EPM. FIGS. 5F - 5I was comparable across all groups in the OF and EPM. FIG. 5J is a graph of the fraction of mice not feeding versus latency to feed in OF in seconds. FIG. 5K is a graph of the latency to feed in OF in seconds of all of the groups of mice. FIG. 5L is a graph of the fraction of mice not feeding versus latency to feed in HC in seconds. FIG. 5M is a graph of food not eaten. FIGS. 5J - 5M show that (R,S)-ketamine (10 mg/kg) and (R,S)-ketamine and prucalopride (10 + 1.5 mg/kg) suppressed hyponeophagia in the NSF but did not alter latency to feed in the home cage or body weight loss. Error bars represent + SEM. * p < 0.05. ** p < 0.01. Sal, saline; K, (R,S)-ketamine; P, prucalopride; CFC, contextual fear conditioning; FST, forced swim test; OF, open field; EPM, elevated plus maze; NSF, novelty suppressed feeding; min, minute; sec, second; mg, milligram; kg, kilogram; cm, centimeter; no, number; HC, home cage; g, gram.

FIG. 6. Combined (R,S)-ketamine and prucalopride does not alter behavioral despair in non-stressed male 129S6/SvEv mice. FIG. 6A shows the behavioral paradigm. Mice were administered saline, (R,S)-ketamine (30 mg/kg), prucalopride (3 mg/kg), or (R,S)-ketamine + prucalopride (10 + 3 mg/kg) one hour prior to FST day 1. FIG. 6B is a graph of immobility in FST day 1. FIG. 6C is a graph of immobility in FST day 2. Immobility time was comparable across all groups on day 1 and day 2 of the FST. Error bars represent + SEM. Sal, saline; K, (R,S)-ketamine; P, prucalopride; hr, hour; FST, forced swim test; sec, second; min, minute; mg, milligram; kg, kilogram.

FIG. 7. Combined (R,S)-ketamine and prucalopride does not alter behavioral despair in non-stressed female 129S6/SvEv mice. FIG. 7A shows the behavioral paradigm. Mice were administered saline, (R,,S)-ketamine (10 mg/kg), prucalopride (1.5 mg/kg), or (R,S)-ketamine + prucalopride (10 + 1.5 mg/kg) one hour prior to FST day 1. FIG. 7B is a graph of immobility in FST day 1. FIG. 7C is a graph of immobility in FST day 2. Immobility time was comparable across all groups on day 1 and day 2 of the FST. Error bars represent + SEM. Sal, saline; K, (R,S)-ketamine; P, prucalopride; hr, hour; FST, forced swim test; sec, second; min, minute; mg, milligram; kg, kilogram.

FIG. 8. Combined (R,S)-ketamine and prucalopride does not alter perseverative behavior in non-stressed male 129S6/SvEv mice. FIG. 8A shows the behavioral paradigm. Mice were administered saline, (R,S)-ketamine (30 mg/kg), prucalopride (3 mg/kg), or (R,S)-ketamine + prucalopride (10 + 3 mg/kg) one hour prior to the OF. FIG. 8B is a graph showing the distance traveled in minutes in the OF. FIG. 8C is a graph showing the total distance traveled in the OF. FIG. 8D is a graph showing the amount of time in the center of the EPM. FIG. 8E is a graph showing the time in the open arms of the EPM. FIG. 8F is a graph showing the number of entries into the open arms of the EPM. FIG. 8G is a graph showing number of marbles buried. FIGS. 8B - 8G show that behavior was comparable across all groups in the OF, EPM and MB assay. FIG. 8H is a graph of the fraction of mice not feeding versus latency to feed in OF. FIG. 8I is a graph of the latency to feed in OF in seconds in all of the groups of mice. FIG. 8J is a graph of body weight loss. FIG. 8K is a graph of the fraction of mice not feeding versus latency to feed in HC. FIG. 8L is a graph of the fraction of mice not feeding versus latency to feed in HC in seconds in all of the groups of mice. FIG. 8M is a graph of food not eaten. FIGS. 8H - 8M show behavior in the NSF was not significantly altered by any drugs assayed. Error bars represent ± SEM. Sal, saline; K, (R,S)-ketamine; P, prucalopride; h, hour; OF, open field; EPM, elevated plus maze; MB, marble burying; NSF, novelty suppressed feeding; cm, centimeter; min, minute; mg, milligram; kg, kilogram; sec, second; no, number; g, gram; HC, home cage.

FIG. 9. Combined (R,S)-ketamine and prucalopride reduces perseverative behavior in non-stressed female 129S6/SvEv mice. FIG. 9A shows the behavioral paradigm. Mice were administered saline, (R,S)-ketamine (10 mg/kg), prucalopride (1.5 mg/kg), or (R,S)-ketamine + prucalopride (10 + 1.5 mg/kg) one hour prior to the OF. FIG. 9B is a graph showing the distance traveled in minutes in the OF. FIG. 9C is a graph showing the total distance traveled in the OF. FIGS. 9B and 9C show that prucalopride (1.5 mg/kg) significantly increased distance travelled in the OF when compared with saline controls. FIG. 9D is a graph showing the amount of time in the center of the EPM. Time spent in the center of the OF was comparable across all groups. FIG. 9E is a graph showing the time in the open arms of the EPM. FIG. 9F is a graph showing the number of entries into the open arms of the EPM. FIGS. 9E and 9F show that no drugs tested altered behavior in the EPM. FIG. 9G is a graph showing number of marbles buried. Mice given prucalopride (1.5 mg/kg) or (R,S)-ketamine + prucalopride (10 + 1.5 mg/kg) significantly reduced the number of marbles buried in the MB assay. FIG. 9H is a graph of the fraction of mice not feeding versus latency to feed in OF. FIG. 9I is a graph of the latency to feed in seconds in the OF in all of the groups of mice. FIG. 9J is a graph of body weight loss. FIG. 9K is a graph of the fraction of mice not feeding versus latency to feed in HC. FIG. 9L is a graph of the fraction of mice not feeding versus latency to feed in HC in seconds in all groups of mice. FIG. 9M is a graph of food not eaten. FIGS. 9H - 9M show behavior was comparable across all groups. Error bars represent ± SEM. * p < 0.05. Sal, saline; K, (R,S)-ketamine; P, prucalopride; h, hour; OF, open field; MB, marble burying; EPM, elevated plus maze; NSF, novelty suppressed feeding; cm, centimeter; min, minute; mg, milligram; kg, kilogram; sec, second; no, number; g, gram; HC, home cage.

### DETAILED DESCRIPTION

### Definitions

The terms used in this specification generally have their ordinary meanings in the art, within the context of this invention and the specific context where each term is used. Certain terms are discussed below, or elsewhere in the specification, to provide additional guidance to the practitioner in describing the methods of the invention and how to use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of the other synonyms. The use of examples anywhere in the specification, including examples of any terms discussed herein, is illustrative only, and in no way limits the scope and meaning of the invention or any exemplified term. Likewise, the invention is not limited to its preferred embodiments.

The term “subject” as used in this application means mammals. Mammals include canines, felines, rodents, bovine, equines, porcines, ovines, and primates. Thus, the compositions and methods disclosed herein can be used in veterinary medicine, e.g., to treat companion animals, farm animals, laboratory animals in zoological parks, and animals in the wild. The disclosure is particularly desirable for human medical applications

The term “patient” as used in this application means a human subject. In some embodiments of the present disclosure, the patient has been exposed to stress or will be exposed to stress or expected to be exposed to stress or at risk for being exposed to stress. In some embodiments, the patient has or is at risk for a stress-induced fear, anxiety, and/or depressive disorder.

The phrase “therapeutically effective amount” or “effective amount” or “therapeutically effective dose” or “effective dose” is used herein to mean an amount sufficient to cause an improvement in a clinically significant condition in the subject, or delays or minimizes or mitigates one or more symptoms associated with the disease or disorder, or results in a desired beneficial change of physiology in the subject.

The terms “treat”, “treatment”, and the like refer to a means to slow down, relieve, ameliorate or alleviate at least one of the symptoms of the disease or disorder, or reverse the disease or disorder after its onset.

The terms “prevent”, “prevention”, and the like refer to acting prior to overt disease or disorder onset, to prevent the disease or disorder from developing or minimize the extent of the disease or disorder or slow its course of development.

The term “in need thereof” would be a subject who has been exposed to stress or will be exposed to stress or expected to be exposed to stress or at risk for being exposed to stress. In some embodiments, the subject is “in need thereof” because they have or are at risk for a stress-induced fear, anxiety, and/or depressive disorder.

The term “agent” as used herein means a substance that produces or is capable of producing an effect and would include, but is not limited to, chemicals, pharmaceuticals, biologics, small organic molecules, antibodies, nucleic acids, peptides, and proteins.

As used herein “an adverse effect” is an unwanted reaction caused by the administration of a drug.

The term “pharmaceutically acceptable derivative” refers to any pharmaceutically acceptable salt, solvate, prodrug, e.g. ester, or other precursors, of a compound which upon administration to the recipient is capable of providing (directly or indirectly) the active compound or an active metabolite or residue thereof. Such salts include pharmaceutically acceptable basic or acid addition salts as well as pharmaceutically acceptable metal salts, ammonium salts and alkylated ammonium salts. Such derivatives are recognizable to those skilled in the art, without undue experimentation. Derivatives are described, for example, in Burger’s Medicinal Chemistry and Drug Discovery, 5th Edition, Vol 1: Principles and Practice, which is incorporated herein by reference. In certain embodiments, pharmaceutically acceptable derivatives include salts, solvates, esters, carbamates, and phosphate esters.

Unless otherwise indicated, all numbers expressing quantities of ingredients, properties and so forth used in the present disclosure and associated claims are to be understood as being modified in all instances by the term “about.” Accordingly, unless indicated to the contrary, the numerical parameters set forth in this disclosure and attached claims are approximations that may vary depending upon the desired properties sought to be obtained by the examples of the present invention. The term “about” or “approximately” means within an acceptable error range for the particular value as determined by one of ordinary skill in the art, which will depend in part on how the value is measured or determined, i.e., the limitations of the measurement system, i.e., the degree of precision required for a particular purpose, such as a pharmaceutical formulation. For example, “about” can mean within 1 or more than 1 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

### Abbreviations

CFC- contextual fear conditioning

FST- forced swim test

OF- open field

EPM- elevated plus maze

NSF- novelty suppressed feeding

HC- housed cage

MB- marble burying

### The Combined Administration of Two Different Agents or Compounds as an Intervention Against Stress-Related Fear, Depressive-Like Behavior and Anxiety-Like Behavior

The current disclosure employs a combined administration of two different compounds or agents, a 5-HT₄R agonist, and ketamine, a ketamine analog, or a pharmaceutically acceptable salt, derivative, or metabolite thereof, an NMDAR antagonist or an AMPAR agonist, as an intervention against stress-related behaviors. At varying dosages, this combination successfully protects against each of stress-related fear, depressive-like behavior, and anxiety-like behavior. While the intervention of the behaviors can be accomplished administering these compounds or agents in one or more compositions, the disclosure herein also provides the basis for a novel compound that will integrate the functional structures of both of the compounds or agents. As such, this disclosure presents a pharmacological intervention against mood and anxiety disorders related to stress.

Stress is a major risk factor for mood and anxiety disorders. However, there is currently no prophylactic drug that intervenes against stress in order to prevent the onset of mental health disorders such as major depressive disorder (MDD) or post-traumatic stress disorder (PTSD). To address this problem, a combined dose of prucalopride and (R,S)-ketamine was administered at different dosage combinations one week prior to contextual fear conditioning (CFC) stress in male 129S6/SvEv mice. Five days later, mice were re-exposed to the training context and then assayed for fear, depressive- and anxiety-like behavior using the forced swim test (FST), open field (OF), elevated plus maze (EPM), and novelty-suppressed feeding (NSF). Prucalopride + (R,S)-ketamine attenuated learned fear. Prucalopride + (R,S)-ketamine protected against depressive-like behavior in the FST and novelty-induced hyponeophagia in the NSF, i.e., anxiety. In particular, the combination of prucalopride and (R,S)-ketamine decreased anxiety, as shown by a decrease in hyponeophagia, when either drug alone had no effect. Additionally, less ketamine could be administered and achieve the same effect when it was combined with prucalopride, in some cases, a third less.

The same protocol used in two separate female mouse lines showed the same results including that the combination of prucalopride and (R,S)-ketamine decreased anxiety, as shown by a decrease in hyponeophagia, when either drug alone had no effect.

Also shown herein was the effect of the administration of different combined doses of prucalopride and (R,S)-ketamine to male mice five minutes after CFC stress. Five days later, mice were re-exposed to the training context and then assayed for fear, depressive- and anxiety-like behavior using the forced swim test (FST), open field (OF), elevated plus maze (EPM), and novelty-suppressed feeding (NSF). Prucalopride + (R,S)-ketamine attenuated learned fear. Prucalopride + (R,S)-ketamine reduced behavioral despair. In particular, the combination of prucalopride and (R,S)-ketamine decreased fear when either drug alone had no effect. Additionally, less ketamine could be administered and achieve the same effect when it was combined with prucalopride, in some cases, a third less.

The same protocol used in female 129S6/SvEv mice showed the same results including that the combination of prucalopride and (R,S)-ketamine attenuated learned fear and reduced behavioral despair. Also the combination decreased anxiety, as shown by a decrease in hyponeophagia, when either drug alone had no effect.

These results indicate that a combined administration of prucalopride, an 5- HT₄R agonist, and (R,S)-ketamine is prophylactic against all three types of stress-induced behaviors in a dose-specific manner, whether the administration is done prior to, or after, exposure to stress.

Lastly, shown herein is that the combined administration of prucalopride and (R,S)-ketamine to female mice not exposed to any stressors, reduced perseverative behavior.

The present compounds, agents or compositions may be administered by various routes, including oral, intravenous (i.v. or IV), intranasal (i.n. or IN), intramuscular (i.m. or IM), caudal, intrathecal, and subcutaneous (s.c.) routes.

### 5-HT₄R Activators or Agonists

The serotonin 4 receptor (5-HT₄R) is a G-protein coupled receptor (GPCR) that activates G protein Gs and stimulates the cAMP/PKA signaling pathway, resulting in the phosphorylation of cAMP response element binding protein (CREB) and as a consequence the expression of a number of genes involved in neuroplasticity (Vidal et al., 2014). The majority of 5-HT₄Rs are expressed in the brain of primates and rodents specifically in the medium spiny neurons of the striatum, the ammon’s horns (Cornu Ammonis 1 (CA1) and CA3) of the hippocampus, the granule cells of the dentate gyrus and glutamatergic neurons in the cortex and amygdala (Rebholz et al., 2018). In addition, 5-HT₄Rs are also found in hypothalamus, ventral pallidum, olfactory bulbs, septal area, and substantia nigra. Mice lacking the 5-HT₄R display anhedonia and a context-dependent anxiety-like behavior (Amigo et al., 2016) and various 5-HT₄R agonists can exert an antidepressant and anxiolytic-like activity (Samuels et al., 2016).

Whether in humans or in rodents, the expression of the 5-HT₄ is found in the limbic regions (mPFC, HPC and NAc). In addition, the basal ganglia, i.e., the caudate nucleus and the lenticular nucleus (putamen and pallidum), the black matter, and the amygdala, also express the 5-HT₄ receptor. The 5-HT₄ receptor is expressed at the somatodendritic level and at the level of the axon terminals of efferent spinal GABAergic neurons of the striatum, the CA1 and CA3 of the hippocampus, the granular cells of the dentate gyrus, and glutamatergic neurons of the cortex, the hippocampus and the amygdala.

5-HT₄ receptor is also found at the peripheral level, in particular at the cardiac level, where activation thereof exerts a positive inotropic effect, at the level of the gastro-intestinal tract where it is involved in intestinal motility, at the level of the adrenal glands where it plays a role in secretion of corticosterone, and at the level of the bladder where it causes contraction of the smooth muscles.

The 5-HT₄ receptor has seven transmembrane domains. The N-terminal region faces towards the extracellular environment, whereas the C-terminal domain, coupled to a Gs protein, faces towards the cytoplasm. The activation of the 5-HT₄ receptor, e.g., by an agonist, can lead to the recruitment of the Gs protein which stimulates adenylate cyclase (AC) which is responsible for the production of cAMP. Protein kinase A (PKA), activated by the cAMP, modulates different ionic currents and in particular potassium currents, the inhibition of which results in neuronal hyperexcitability. The PKA is also capable of phosphorylating the protein binding the response element to the cAMP (CREB - cAMP response element binding protein), which results in an increase in the transcription of neurotrophic brain factor (BDNF, brain-derived neurotrophic factor), involved in cognition, mood and cell survival.

The term “agonist” may refer to a substance, an agent or a compound capable of binding to and activating one or more receptors, such as 5-HT₄R. The term “agonist” may refer to a compound having the ability to initiate or enhance a biological function of a target protein (e.g., one or more receptors, such as 5-HT₄R), whether by enhancing or initiating the activity or expression of the target protein. 5-HT₄R agonists may be compounds that activate the action of the 5-HT4 receptor. The term “agonist” may be defined in the context of the biological role of the target protein. In one embodiment, an agonist is an agent that binds to a receptor (e.g., 5-HT₄R) and activates the receptor to produce a biological response. While agonists provided herein can specifically interact with (e.g., bind to) the target protein, compounds that initiate or enhance a biological activity of the target protein by interacting with other members of the signal transduction pathway of which the target protein is a member are also specifically included within this definition. A 5-HT₄R agonist may be a compound or an agent that activates the action of 5-HT₄R. A 5-HT₄R agonist may be any agent that acts directly or indirectly through or upon 5-HT₄R to produce a pharmacological effect. The terms “agonist of 5-HT₄R”, “agonist of the 5-HT4 receptor”, “5-HT4 receptor agonist”, and “5-HT₄R agonist” are used interchangeably herein.

The 5-HT₄R agonist may be selective for 5-HT4 receptors or it may be non-selective, exhibiting agonist or antagonist activity at other serotonin receptors. In one embodiment, the 5-HT₄R agonist is selective for 5-HT4 receptors.

The 5-HT₄R agonists may include full agonists, partial agonists, or mixed 5-HT₄R agonists/antagonists.

“Full agonists” may refer to agents bind to and activate a receptor with the maximum response that an agonist can elicit at the receptor. An agent may act as a full agonist in some tissues and as a partial agonist in other tissues, depending upon the relative numbers of receptors and differences in receptor coupling.

“Partial agonists” may refer to compounds able to bind and activate a given receptor, but having only partial efficacy at the receptor relative to a “full agonist” or complete agonist. Partial agonists can act as antagonists when competing with a full agonist for receptor occupancy and producing a net decrease in the receptor activation compared to the effects or activation observed with the full agonist alone. Partial agonists may refer to mixed agonists/antagonists, which differentially affect a receptor function within different dose ranges. For example, partial agonists may serve as agonists at lower doses, and as antagonists at higher doses. Partial agonists may be compounds that have reduced efficacy for inducing conformational change in receptors (typically 40-80%) relative to full agonists, and which may induce agonist effects at low dose but antagonist effects at high dose.

The 5-HT₄R agonist may be an indole, a benzamide, a benzoate, an arylketone or a benzamide.

Non-limiting examples of 5-HT₄R agonists include, 1-(4-amino-5-chloro-2-methoxyphenyl)-3-[1(n-butyl)-4-piperidinyl]-1-propanone HCl (RS-67,333 or RS67333), 4-amino-5-chloro-2,3-dihydro-N-[1-3-methoxypropyl)-4-piperidinyl]-7-benzofuran carboxamide monohydrochloride (prucalopride), 4-[4-[4-Tetrahydrofuran-3-yloxy)-benzo[d]isoxazol-3-yloxymethyl]-piperidin-1-ylmethyl]-tetrahydropyran-4-ol (PF-04995274), and combinations thereof.

Non-limiting examples of 5-HT₄R agonists also include, 2-[1-(4-Piperonyl)piperazinyl]benzothiazole (PPB), 5-methoxytryptamine, PRX-03140, cisapride ((±)-cis-4-amino-5-chloro-N-[1-[3-(4-fluorophenoxy)propyl]-3-methoxy-4-piperidinyl]-2-methoxybenzamide monohydrate), BIMU-8 (2,3-Dihydro-N-[(3-endo)-8-methyl-8-azabicyclo[3.2.1]oct-3-yl]-3-(1-methylethyl)-2-oxo-1H-benzimidazole-1-carboxamide, RS67506 (methylsulphonylamino)ethyl-4-piperidinyl] -1-propanone hydrochloride), mosapride (4-amino-5-chloro-2-ethoxy-N-[[4-[(4-fluorophenyl)methyl]-2-morpholinyl]methyl]benzamide citrate), tegaserod (2-[(5-Methoxy-1H-indol-3-yl)methylene]-N-pentyl-hydrazinecarboximidamide maleate), ML10302 (4-Amino-5-chloro-2-methoxybenzoic acid 2-(1-piperidinyl)ethyl ester hydrochloride), velusetrag (TD-5108) (N-[(1R,3R,5S)-8-[(2R)-2-hydroxy-3-(N-methylmethanesulfonamido)propyl]-8-azabicyclo[3.2.1]octan-3-yl]-2-oxo-1-(propan-2-yl)-1,2-dihydroquinoline-3-carboxamide), naropride (ATI-7505) ([(3R)-1-azabicyclo[2.2.2]octan-3-yl] 6-[(3S,4R)-4-[(4-amino-5-chloro-2-methoxybenzoyl)amino]-3-methoxypiperidin-1-yl]hexanoate, cinitapride (4-amino-N-[1-(cyclohex-3-en-1-ylmethyl)piperidin-4-yl]-2-ethoxy-5-nitrobenzamide), metoclopramide (4-amino-5-chloro-N-(2-(diethylamino)ethyl)-2-methoxybenzamide), renzapride (ATL-1251, BRL 24924, (±)-endo-4-amino-5-chloro-2-methoxy-N-(1-azabicyclo [3.3.1]non-4-yl) benzamide), RQ-00000010 (4-{[4-({[4-(2,2,2-trifluoroethoxy)-1,2-benzisoxazol-3-yl]oxy}methyl)piperidin-1-yl]methyl}tetrahydro-2H-pyran-4-carboxylic acid), SUVN-D4010 (1-isopropyl-3-{5-[1-(3-methoxy propyl) piperidin-4-yl]-[1,3,4]oxadiazol-2-yl}-1H-indazole), TD-8954 (4-{(4-[(2-isopropyl-1H-benzoimidazole-4-carbonyl)amino]methyl}- piperidin-1-ylmethyl)piperidine-1-carboxylic acid methyl ester), SC53116 (4-Amino-5-chloro-N-[[(1S,7aS)-hexahydro-1H-pyrrolizin-1-yl]methyl]-2-methoxy-benzamide), BIMU-1 (3-ethyl-2,3-dihydro-N-(8-methyl-8-azabicyclo[3.2.1]oct-3-yl)-2-oxo-1H- benzimidazole-1-carboxamide hydrochloride), donecopride (MR31147, which is: 1-(4-amino-5-chloro-2-methoxyphenyl)-3-[1-(cyclohexylmethyl)-4-piperidinyl]propan-1-one), LS 650155 (Caeserod, which is: 5-(8-amino-7-chloro-2,3-dihydrobenzo[b][1,4]dioxin-5-yl)-3-(1-phenethylpiperidin-4-yl)-1,3,4-oxadiazol-2(3H)-one hydrochloride), PF-00885706, N-[2-[(1R,8S)-4-[[4-(cyclobutylamino)-5-(trifluoromethyl)pyrimidin-2-yl] amino]-1 1-azatricyclo[6.2.1.02,7]undeca-2(7),3,5-trien-11-yl]-2-oxoethyl]acetamide, and combinations thereof.

RS-67,333 is a high-affinity 5-HT₄R partial agonist (Eglen et al., 1995). This drug is effective in improving behavioral deficits, decreasing the number of amyloid plaques as well as level of amyloid beta (Aβ) species, and decreasing hippocampal astrogliosis and microgliosis in the 5xFAD mouse model of Alzheimer’s disease (AD) (Giannoni et al., 2013). RS67333 is an arylketone. Incorporating an n-butyl group on the piperidine has increased the agonist activity with great effectiveness, optimal selectivity, and excellent bioavailability. Its increased hydrophobicity helps pass the blood-brain barrier, allowing for penetration into the brain (Eglen et al. 1995).

Prucalopride is a selective, high affinity 5-HT₄R agonist (Prins et al., 1999). Prucalopride is a derivative of the family of benzofurans which exhibits increased selectivity for 5-HT₄ receptor but no affinity for the hERG (human Ether-a-go-go Related Gene) channels. In 2018, it was approved by the FDA for chronic constipation and is currently being tested for chronic intestinal pseudo-obstruction. Prucalopride has also been tested in two separate clinical trials to investigate its effects on emotional processing in health volunteers after an acute (e.g., single dose) or chronic (e.g., 1 week) administration (Morris et al., 2017; Zanos and Gould, 2018).

PF-04995274 is a potent, partial 5-HT₄R agonist (Grimwood et al., 2011). A clinical trial was conducted to evaluate PF-04995274, alone or in combination with donepezil, on scopolamine-induced deficits in psychomotor and cognitive function in healthy adults. However, this trial was terminated, but not due to safety concerns. Currently, a clinical trial is underway to test whether adjunctive administration of PF-04995247 has positive effects on emotional processing and neural activity in mediated, treatment-resistant (TRD) depressed patients compared to placebo (Morris et al., 2017).

Tegaserod is a partial agonist of the 5-HT₄R, with moderate affinity for the 5-HT₁ (agonist) and 5-HT_{2A-C} (antagonist) receptors.

Cisapride is a parasympathomimetic which, by activating the 5-HT₄R, increases the acetylcholine liberated in the enteric nervous system.

Cinitapride is a benzamide which acts as a 5-HT_{1A} and 5-HT₄ receptor agonist, and a 5-HT_{2A} receptor antagonist.

Mosapride is a selective 5-HT₄R agonist, the main active metabolite of which acts as a 5-HT₃ receptor antagonist.

Metoclopramide is a 5-HT₄ and 5-HT_{3A} receptor agonist. It is a D2 receptor antagonist. It is also an M1 muscarinic receptor agonist, and an acetylcholinesterase inhibitor.

SUVN-D4010 is a powerful, selective and effective 5-HT4R partial agonist, having good bioavailability via the oral route.

Mixed 5-HTR agonists/antagonists include, but are not limited to, buspirone, mianserin, trazodone, and mirtazapine.

The terms “serotonin,” “5-hydroxytryptamine” and “5-HT” refers to a phenolic amine neurotransmitter produced from tryptophan by hydroxylation and decarboxylation in serotonergic neurons of the central nervous system and enterochromaffin cells of the gastrointestinal tract. Serotonin is a precursor of melatonin.

### Ketamine

Ketamine ((RS)-2-(2-chlorophenyl)-2-(methylamino)cyclohexanone) is an antagonist of the glutamate N-methyl-D-aspartate (NMDA) receptor (NMDAR). Ketamine also acts on opioid receptors, sigma receptors, muscarinic receptors, and monoamine transporters.

Ketamine is a chiral compound. As used herein, the term “ketamine” may refer to (S)-ketamine (also referred to as S(+)-ketamine or esketamine), (R)-ketamine (R(-)-ketamine), or a racemic mixture of (S)-ketamine and (R)-ketamine. In certain embodiments, the ketamine compositions contain different proportions of the S(+) and R(-) stereoisomers. In certain embodiments, the ketamine compositions contain only (S)-ketamine or (R)-ketamine or are enantiomerically enriched for a ketamine enantiomer. In certain embodiments, the ketamine composition is enriched to contain, for example, greater than 60%, greater than 70%, greater than 80%, greater than 90%, greater than 95%, greater than 99%, or greater than 99.9 of (S)-ketamine or (R)-ketamine. See Paul et al., 2009; Paskalis et al., 2010; Noppers et al., 2011; Matthews et al., 2012 and International Patent Publication No. WO2013/138322.

Ketamine is a derivative of arylcyclohexylamineand contains a chiral center. Since the 1950s, a large number of arylcyclohexylamines have been synthesized: these compounds have shown a wide range of possible pharmacological activities. When administered orally, it undergoes first-pass metabolism, where it is stereo selectively metabolized into a broad array of metabolites, including norketamine, hydroxyketamines, dehydronorketamine and hydroxynorketamine (HNK). After ketamine administration, (2S,6S;2R,6R)-HNK are the two major HNK metabolites found in the plasma and brain. Interestingly, a study has shown that the (2R,6R)-HNK metabolite is: 1) essential for the antidepressant effects of ketamine; 2) dependent on α-amino-3-hydroxy-5-methyl-4-isoxazolepropionic acid (AMPA) receptor activation; and 3) non-hypnotic (Zanos et al., 2016). All of these compounds are expected to behave similarly in the presently described methods, including enantiomers and non-psychotomimetic metabolites of ketamine.

The present disclosure also encompasses ketamine’s enantiomers and non-psychotomimetic metabolites. Such compounds include:
(2R,6R)-HNK, a metabolite of ketamine that may mediate the antidepressant effects of ketamine and lacks the ketamine-related side effects (Zanos et al., 2016);
(2S,6S)-HNK, a metabolite of ketamine (synthesis of these compounds (2R,6R)-HNK and(2S,6S)-HNK are described in Zanos et al., 2016 and WO 2013/056229. The use of (2R,6R)-hydroxynorketamine, (S)-dehydronorketamine and other stereoisomeric dehydro and hydroxylated metabolites of (R,S)-ketamine in the treatment of depression and neuropathic pain);
(R)-ketamine, the R-enantiomer of ketamine, which has rapid-onset and sustained antidepressant effects without psychotomimetic side effects (Yang et al., 2015); and
(S)-ketamine, the S-enantiomer of ketamine, which is being developed as an intranasal spray, currently in phase III clinical trials for treatment-resistant depression.

Finally, other ketamine analogs are also expected to be protective. Such compounds include:
Fluorodeschloroketamine, an analog of ketamine where the chlorine (Cl) group has been replaced by fluorine (F); and
Tiletamine, an analog of ketamine commonly used as a veterinary anesthetic.

### NMDA Receptor Antagonists—Ketamine and Other Compounds

NMDA receptor antagonists are compounds that antagonize, or inhibit, the action of the NMDA receptor. An NMDA receptor antagonist may be a competitive antagonist, an uncompetitive antagonist, a noncompetitive antagonist, and/or a glycine antagonist.

Non-limiting examples of NMDA receptor antagonists include, ketamine, dextromethorphan (DXM), histogranin, memantine, meperidine, methadone, methoxetamine (MXE), phencyclidine (PCP), nitrous oxide (N₂O), AP5 (APV, R-2-amino-5-phosphonopentanoate), AP7 (2-amino-7-phosphonoheptanoic acid), CPPene ((3-[(R)-2-carboxypiperazin-4-yl]-prop-2-enyl-l-phosphonic acid), Selfotel, Amantadine, Atomoxetine, AZD6765, Agmatine, chloroform, dextrallorphan, dextromethorphan, dextrorphan, diphenidine, dizocilpine (MK-801), ethanol, eticyclidine, gacyclidine, ibogaine, magnesium, memantine, nitromemantine, rolicyclidine, tenocyclidine, methoxydine, tiletamine, neramexane, eliprodil, dexoxadrol, etoxadrol, remacemide, delucemine, WMS-2539, NEFA, 8A-PDHQ, HU-211, Aptiganel (Cerestat, CNS-1102), rhynchophylline, kynurenic acid, Rapastinel (GLYX-13), NRX-1074, 7-Chlorokynurenic acid, 4-Chlorokynurenine (AV-101), TK-40, 1-Aminocyclopropanecarboxylic acid (ACPC), L-Phenylalanine, Xenon, or analogs or derivatives thereof. Ketamine derivatives such as Rapastinel or Glyx-13 are also included. Rapastinel is an NMDA receptor glycine site partial agonist. It is an amidated tetrapeptide (Thr—Pro—Pro—Thr—NH₂) which rapidly crosses the blood brain barrier, but is not active orally.

Compounds that are mechanistically similar to ketamine are expected to be protective against stress-induced de novo psychopathology. Such compounds include:
Ro 25-6981, a GluN2B-selective antagonist, which has been shown to have rapid antidepressant actions in rodent models of depression;
CP-101,606, a GluN2B-selective antagonist: A placebo-controlled trial of the NR2B specific NMDA antagonist CP-101, 606 plus paroxetine for treatment resistant depression (TRD);
GLYX-13, a novel N-methyl-D-aspartate receptor (NMDAR) glycine-site functional partial agonist and rapid-acting antidepressant. GLYX-13 received Breakthrough Therapy designation from the U.S. Food and Drug Administration (FDA) for adjunctive treatment of MDD in January, 2016; and
CX546 (Tocris), an ampakine (an AMPA receptor agonist), which relieves the respiratory depression induced by fentanyl.

Non-limiting examples of the NMDA receptor antagonists also include anti-receptor antibodies, and anti-ligand antibodies.

Several synthetic opioids function as NMDA receptor-antagonists, such as pethidine, methadone, meperidine, dextropropoxyphene, tramadol, levorphanol, and ketobemidone.

### AMPA Receptor Agonists

AMPA receptor agonists are compounds that activate the action of the α-amino-3-hydroxy-5-methyl-4-isoxazolepropionic acid (AMPA) receptor. It is expected that compounds that activate the AMPA receptor, including metabolites, will have a similar effect as the present effects shown with ketamine, in view of findings that a ketamine metabolite’s antidepressant activity in mice was due to sustained activation of the AMPA receptor, rather than inhibiting NMDAR (Zanos et al.,2016).

Thus, in certain embodiments, AMPA receptor agonists may be used in the methods described herein. Non-limiting examples of the AMPA receptor agonists include glutamate, AMPA, 5-fluorowillardiine, domoic acid, quisqualic acid, (2R,6R)-hydroxynorketamine, and CX546, as well as pharmaceutically acceptable salts, derivatives, or metabolites thereof.

### Pharmaceutical Compounds

The 5-HT₄R agonists, ketamine, NMDAR antagonists and AMPAR agonists used in the present methods include all hydrates, solvates, and complexes of the compounds described herein. If a chiral center or another form of an isomeric center is present in a present compound, all forms of such isomer or isomers, including enantiomers and diastereomers, are intended to be covered herein. Compounds containing a chiral center may be used as a racemic mixture, an enantiomerically enriched mixture, or the racemic mixture may be separated using well-known techniques and an individual enantiomer may be used alone. The compounds described in the present disclosure may be in racemic form or as individual enantiomers. The enantiomers can be separated using known techniques, such as those described in IUPAC (1997) Pure and Applied Chemistry 69:1469-1474. In cases in which compounds have unsaturated carbon-carbon double bonds, both the cis (Z) and trans (E) isomers are within the scope of this disclosure. In cases wherein compounds may exist in tautomeric forms, such as keto-enol tautomers, each tautomeric form is contemplated as being included within this disclosure whether existing in equilibrium or predominantly in one form.

When the structure of the compounds used in this disclosure includes an asymmetric carbon atom such compound can occur as racemates, racemic mixtures, and isolated single enantiomers. All such isomeric forms of these compounds are expressly included in this disclosure. Each stereogenic carbon may be of the R or S configuration. It is to be understood accordingly that the isomers arising from such asymmetry (e.g., all enantiomers and diastereomers) are included within the scope of this disclosure, unless indicated otherwise. Such isomers can be obtained in substantially pure form by classical separation techniques and by stereochemically controlled synthesis, such as those described in “Enantiomers, Racemates and Resolutions” by J. Jacques, A. Collet and S. Wilen, Pub. John Wiley & Sons, NY, 1981. For example, the resolution may be carried out by preparative chromatography on a chiral column.

The present disclosure is also intended to include use of all isotopes of atoms occurring on the compounds disclosed herein. Isotopes include those atoms having the same atomic number but different mass numbers. Isotopically-labeled compounds can generally be prepared by conventional techniques known to those skilled in the art or by processes analogous to those described herein using appropriate isotopically-labeled reagents in place of the non-labeled reagents employed.

The compounds of the instant disclosure may be in a salt form. As used herein, a “salt” is a salt of the instant compound which has been modified by making acid or base, salts of the compounds. In the case of compounds used for treatment of mammals, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately treating a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The present methods also encompass administering a physiologically functional derivative of the present compound. As used herein, the term “physiologically functional derivative” refers to a compound (e.g., a drug precursor) that is transformed in vivo to yield the present compound or its active metabolite, or a pharmaceutically acceptable salt, hydrate or solvate of the compound. The transformation may occur by various mechanisms (e.g., by metabolic or chemical processes), such as, for example, through hydrolysis in blood. Prodrugs are such derivatives, and a discussion of the use of prodrugs is provided by T. Higuchi and W. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987.

### Pharmaceutical Compositions

While it is possible that the 5-HT₄R agonists, ketamine, a ketamine analog, or a pharmaceutically acceptable salt, derivative, or metabolite thereof, NMDAR antagonists and AMPAR agonists, as well as salts, solvates and physiological functional derivatives thereof, may be administered as the raw chemical, it is possible to present the active ingredient as a pharmaceutical composition. Accordingly, the disclosure further provides a pharmaceutical composition, which comprises the present agent or compound and/or salts, solvates and physiological functional derivatives thereof, and one or more pharmaceutically acceptable carriers, diluents, or excipients. The carrier(s), diluent(s) or excipient(s) must be acceptable in the sense of being compatible with the other ingredients of the formulation and not deleterious to the recipient thereof. In accordance with another aspect of the disclosure there is also provided a process for the preparation of a pharmaceutical composition including admixing the present compound, or salts, solvates and physiological functional derivatives thereof, with one or more pharmaceutically acceptable carriers, diluents or excipients.

The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound or compounds, and pharmaceutically acceptable excipients.

Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

As used herein, the phrase “pharmaceutically acceptable” refers to molecular entities and compositions that are “generally regarded as safe”, e.g., that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopoeia or other generally recognized pharmacopeias for use in animals, and more particularly in humans.

Pharmaceutical compositions of the present disclosure may be presented in unit dose forms containing a predetermined amount of active ingredient per unit dose. Such a unit may contain, for example, 5 µg to 1 g, preferably 1 mg to 700 mg, more preferably 5 mg to 100 mg of the present compound, depending on the condition being treated, the route of administration and the age, weight and condition of the patient. Such unit doses may therefore be administered more than once a day. Preferred unit dosage compositions are those containing a daily dose or sub-dose (for administration more than once a day), as herein above recited, or an appropriate fraction thereof, of an active ingredient. Furthermore, such pharmaceutical compositions may be prepared by any of the methods well known in the pharmacy art.

Pharmaceutical compositions of the disclosure may be adapted for administration by any appropriate route, for example by the oral (including buccal or sublingual), inhaled, nasal, ocular, or parenteral (including intravenous and intramuscular) route. The present composition may be injected. Such compositions may be prepared by any method known in the art of pharmacy, for example by bringing into association the active ingredient with the carrier(s) or excipient(s).

In a further embodiment, the present disclosure provides a pharmaceutical composition adapted for administration by the oral route.

Pharmaceutical compositions of the present disclosure which are adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

Capsules are made by preparing a powder mixture, as described above, and filling formed gelatin sheaths. Glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, suitable binders, lubricants, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free-flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, syrups and elixirs can be prepared in dosage unit form so that a given quantity contains a predetermined amount of the compound. Syrups can be prepared by dissolving the compound in a suitably flavored aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

It should be understood that, in addition to the ingredients particularly mentioned above, the compositions may include other agents conventional in the art having regard to the type of formulation in question, for example those suitable for oral administration may include flavoring agents.

A further preferred form of administration is parenteral including intravenous administration. Pharmaceutical compositions adapted for parenteral administration, including intravenous administration, include aqueous and non-aqueous sterile injectable solutions or suspensions, which may contain anti-oxidants, buffers, bacteriostats, and solutes that render the compositions substantially isotonic with the blood of the subject. Other components which may be present in such compositions include water, alcohols, polyols, glycerine, and vegetable oils. Compositions adapted for parental administration may be presented in unit-dose or multidose containers, such as sealed ampules and vials, and may be stored in a freeze-dried (lyophilized) condition requiring only the addition of a sterile carrier, immediately prior to use. Extemporaneous injection solutions and suspensions may be prepared from sterile powders, granules, and tablets. Suitable vehicles that can be used to provide parenteral dosage forms of the invention are well known to those skilled in the art. Examples include: water for Injection USP; aqueous vehicles such as Sodium Chloride Injection, Ringer’s Injection, Dextrose Injection, Dextrose and Sodium Chloride Injection, and Lactated Ringer’s Injection; water-miscible vehicles such as ethyl alcohol, polyethylene glycol, and polypropylene glycol; and non-aqueous vehicles such as corn oil, cottonseed oil, peanut oil, sesame oil, ethyl oleate, isopropyl myristate, and benzyl benzoate.

Further methods of administration include mucosal, such as nasal, sublingual, vaginal, buccal, or rectal; or transdermal administration to a subject.

Pharmaceutical compositions adapted for nasal and pulmonary administration may comprise solid carriers such as powders, which can be administered by rapid inhalation through the nose. Compositions for nasal administration may comprise liquid carriers, such as sprays or drops. Alternatively, inhalation directly through into the lungs may be accomplished by inhalation deeply or installation through a mouthpiece. These compositions may comprise aqueous or oil solutions of the active ingredient. Compositions for inhalation may be supplied in specially adapted devices including, but not limited to, pressurized aerosols, nebulizers or insufflators, which can be constructed so as to provide predetermined dosages of the active ingredient.

### Dosages

In certain embodiments, the effective or therapeutically effective amount or dose of the 5-HT₄R agonist, ketamine, a ketamine analog, or a pharmaceutically acceptable salt, derivative, or metabolite thereof, NMDAR antagonist and AMPAR agonist is about 0.01 to about 40 mg per kilogram of body weight of the subject (mg/kg), i.e., from about 0.01 mg/kg to about 40 mg/kg body weight.

In certain embodiments, the effective or therapeutically effective amount or dose of the 5-HT₄R agonist, ketamine, ketamine analog, or a pharmaceutically acceptable salt, derivative, or metabolite thereof, NMDAR antagonist and AMPAR agonist ranges from about 0.01 to about 40 mg/kg body weight, from about 0.01 to about 35 mg/kg body weight, from about 0.01 to about 30 mg/kg body weight, from about 0.01 to about 25 mg/kg body weight, from about 0.01 to about 20 mg/kg body weight, from about 0.01 to about 15 mg/kg body weight, from about 0.01 to about 10 mg/kg body weight, from about 0.01 to about 5 mg/kg body weight, from about 0.01 mg/kg to about 3 mg/kg body weight, from about 0.01 to about 2 mg/kg of body weight, from about 0.01 to about 1.5 mg/kg of body weight, or from about 0.01 to about 1 mg/kg of body weight.

In certain embodiments, the effective or therapeutically effective amount or dose of the 5-HT₄R agonist, ketamine, ketamine analog, or a pharmaceutically acceptable salt, derivative, or metabolite thereof, NMDAR antagonist and AMPAR agonist ranges from about 0.5 to about 40 mg/kg body weight, from about 0.5 to about 35 mg/kg body weight, from about 0.5 to about 30 mg/kg body weight, from about 0.5 to about 25 mg/kg body weight, from about 0.5 to about 20 mg/kg body weight, from about 0.5 to about 15 mg/kg body weight, from about 0.5 to about 10 mg/kg body weight, from about 0.5 to about 5 mg/kg body weight, from about 0.5 mg/kg to about 3 mg/kg body weight, from about 0.5 to about 2 mg/kg of body weight, from about 0.5 to about 1.5 mg/kg of body weight, or from about 0.5 to about 1 mg/kg of body weight.

In certain embodiments, the effective or therapeutically effective amount or dose of the 5-HT₄R agonist, ketamine, ketamine analog, or a pharmaceutically acceptable salt, derivative, or metabolite thereof, NMDAR antagonist and AMPAR agonist ranges from about 1 to about 40 mg/kg body weight, from about 1 to about 35 mg/kg body weight, from about 1 to about 30 mg/kg body weight, from about 1 to about 25 mg/kg body weight, from about 1 to about 20 mg/kg body weight, from about 1 to about 15 mg/kg body weight, from about 1 to about 10 mg/kg body weight, from about 1 to about 5 mg/kg body weight, from about 1 mg/kg to about 3 mg/kg body weight, or from about 1 to about 2 mg/kg of body weight.

In certain embodiments, the effective or therapeutically effective amount or dose of the 5-HT₄R agonist, ketamine, ketamine analog, or a pharmaceutically acceptable salt, derivative, or metabolite thereof, NMDAR antagonist and AMPAR agonist ranges from about 5 to about 40 mg/kg body weight, from about 5 to about 35 mg/kg body weight, from about 5 to about 30 mg/kg body weight, from about 5 to about 25 mg/kg body weight, from about 5 to about 20 mg/kg body weight, from about 5 to about 15 mg/kg body weight, or from about 5 to about 10 mg/kg body weight.

In certain embodiments, the effective or therapeutically effective amount or dose of the 5-HT₄R agonist, ketamine, ketamine analog, or a pharmaceutically acceptable salt, derivative, or metabolite thereof, NMDAR antagonist and AMPAR agonist ranges from about 10 to about 40 mg/kg body weight, from about 10 to about 35 mg/kg body weight, from about 10 to about 30 mg/kg body weight, from about 10 to about 25 mg/kg body weight, from about 10 to about 20 mg/kg body weight, or from about 10 to about 15 mg/kg body weight.

In certain embodiments, the effective or therapeutically effective amount or dose is about 0.01 mg/kg, about 0.05 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, about 2.0 mg/kg, about 3.0 mg/kg body weight, about 5.0 mg/kg body weight, about 10.0 mg/kg body weight, about 15.0 mg/kg body weight, about 20.0 mg/kg body weight, about 25.0 mg/kg body weight, about 30.0 mg/kg body weight, about 35.0 mg/kg body weight, or about 40.0 mg/kg body weight,

In certain embodiments, the effective or therapeutically effective amounts or doses listed above are the amounts of the individual 5-HT₄R agonist, ketamine, ketamine analog, or a pharmaceutically acceptable salt, derivative, or metabolite thereof, NMDAR antagonist and AMPAR agonist. In some embodiments, the effective or therapeutically effective amounts or doses listed above are the total amounts of the total 5-HT₄R agonist and ketamine, ketamine analog, or a pharmaceutically acceptable salt, derivative, or metabolite thereof, NMDAR antagonist or AMPAR agonist in combination. For example, an effective or therapeutically effective amount or dose can comprise about 0.01 to about 40 mg per kilogram of a 5-HT₄R agonist and about 0.01 to about 40 mg per kilogram of ketamine. In a further example, an effective or therapeutically effective amount or dose can comprise about 0.01 to about 40 mg per kilogram total of a 5-HT₄R agonist combined with ketamine.

In certain embodiments, the dose of the present composition or compositions per administration is from about 1 to about 250 mg, about 10 mg to about 300 mg, about 10 mg to about 250 mg, about 10 to about 200 mg, about 15 to about 175 mg, about 20 to about 175 mg, about 8 mg to about 32 mg, about 50 mg to about 75 mg, about 25 to about 150 mg, about 25 to about 125 mg, about 25 to about 100 mg, about 50 to about 100 mg, about 50 mg to about 75 mg, about 75 mg to about 100 mg, or about 75 mg to about 200 mg, or about 100 mg to about 300 mg, or about 100 mg to about 400 mg, or about 250 mg to about 500 mg.

In certain embodiments, the dose of the present composition or compositions per administration is about 1 mg, 2 mg, 4 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 300 mg, 350 mg, 400 mg, 450 mg, and 500 mg.

In certain embodiments, a single composition contains or comprises both active agents or compounds, i.e., 5-HT₄R agonist and ketamine, ketamine analog, or a pharmaceutically acceptable salt, derivative, or metabolite thereof, NMDAR antagonist or AMPAR agonist are in a single composition. In some embodiments, the active agents or compounds are in different compositions. In certain embodiments, an effective amount of the ketamine is a sub-anesthetic amount of ketamine, or a pharmaceutically acceptable salt or solvate thereof, or a physiologically functional derivative thereof.

In certain embodiments, the effective or therapeutically effective amount or dose is below the level that results in one or more side effects of the agent.

In certain embodiments, the effective or therapeutically effective amount or dose may be adjusted depending on conditions of the disease/disorder to be treated or prophetically treated, the age, body weight, general health conditions, sex, and diet of the subject, dose intervals, administration routes, excretion rate, and combinations of drugs.

An initial dose may be larger, followed by one or more smaller maintenance doses. Other ranges are possible, depending on the subject’s response to the treatment. An initial dose may be the same as, or lower or higher than subsequently administered doses.

The agents, compounds, composition or compositions may be administered daily, weekly, biweekly, several times daily, semi-weekly, every other day, bi-weekly, quarterly, several times per week, semi-weekly, monthly, or more. The duration and frequency of treatment may depend upon the subject’s response to treatment.

In certain embodiments, a subject may be administered 1 dose, 2 doses, 3 doses, 4 doses, 5 doses, 6 doses or more of the present agents, compounds, composition or compositions. In certain embodiments, a single dose of the present agents, compounds, composition or compositions is administered in the present method. In certain embodiments, multiple doses of the present agents, compounds, composition or compositions (e.g., 2 doses, 3 doses, 4 doses, 5 doses, 6 doses, 7 doses, 8 doses, 9 doses, 10 doses or more) are administered in the present method.

In certain embodiments, when there are more than one doses of the present agents, compounds, composition or compositions administered to a subject, the second dose is lower than the first dose. In certain embodiments, the second dose is an amount that is at most one-half, one-quarter, or one-tenth the amount of the first dose.

The number and frequency of doses may be determined based on the subject’s response to administration of the agents, compounds, composition or compositions, e.g., if one or more of the patient’s symptoms improve and/or if the subject tolerates administration of the composition without adverse reaction.

In certain embodiments, the agents, compounds present composition or compositions is administered at least once a day, at least twice a day, at least three times per day, or more. In certain embodiments, the agents, compounds, composition or compositions is administered at least once a week, at least twice a week, at least three times per week, or more frequently. In certain embodiments, the present composition or compositions is administered at least twice per month, or at least once per month.

Treatment using the present method can continue as long as needed.

### Dosing Time Frame

In certain embodiments, the 5-HT₄R agonist, and ketamine, ketamine analog, or a pharmaceutically acceptable salt, derivative, or metabolite thereof, NMDAR antagonist or AMPAR agonist or the composition or compositions comprising them, is administered to a subject prior to a stressor. In certain embodiments, the 5-HT₄R agonist, and ketamine, ketamine analog, or a pharmaceutically acceptable salt, derivative, of metabolite thereof, NMDAR antagonist or AMPAR agonist or the composition or compositions comprising them is administered to a subject both prior to and after a stressor. In certain embodiments, the 5-HT₄R agonist, and ketamine, ketamine analog, or a pharmaceutically acceptable salt, derivative, or metabolite thereof, NMDAR antagonist or AMPAR agonist or the composition or compositions comprising them is administered to a subject after a stressor. In certain embodiments, the 5-HT₄R agonist, and ketamine, ketamine analog, or a pharmaceutically acceptable salt, derivative, or metabolite thereof, NMDAR antagonist or AMPAR agonist or the composition or compositions comprising them is administered to a subject prior to a stressor, and again prior to a recurrence of the stressor or a different stressor.

In certain embodiments, the 5-HT₄R agonist, and ketamine, ketamine analog, or a pharmaceutically acceptable salt, derivative, or metabolite thereof, NMDAR antagonist or AMPAR agonist or the composition or compositions comprising them is administered to the subject about 1 hour to about 5 hours, about 1 hour to about 1 day, about 5 hours to about 10 hours, about 10 hours to about 12 hours, 12 hours to about 1 day, 12 hours to about 4 weeks, about 18 hours to about 4 weeks, about 1 day to about 3.5 weeks, about 2 days to about 3 weeks, about 3 days to about 3 weeks, about 4 days to about 3 weeks, about 5 days to about 3 weeks, about 6 days to about 3 weeks, about 2 days to about 2.5 weeks, about 3 days to about 2.5 weeks, about 4 days to about 2.5 weeks, about 5 days to about 2.5 weeks, about 6 days to about 2.5 weeks, about 1 week to about 2.5 weeks, about 1 week to about 2.5 weeks, about 1 week to about 2 weeks, about 5 minutes to about 3 days, about 10 minutes to about 2 days, about 15 minutes to about 24 hours, about 20 minutes to about 12 hours, about 30 minutes to about 8 hours, about 45 minutes to about 5 hours, about 1 hour to about 12 hours, about 2 hours to about 5 hours, about 5 minutes, about 10 minutes, about 15 minutes, about 20 minutes, about 30 minutes, about 45 minutes, about 1 hour, about 2 hours, about 3 hours, about 4 hours, about 5 hours, about 6 hours, about 8 hours, about 10 hours, about 12 hours, about 15 hours, about 1 day, about 1.5 days, about 2 days, about 3 days, about 4 days, about 5 days, about 6 days, about 1 week, about 8 days, about 9 days, about 10 days, about 11 days, about 12 days, about 13 days, about 2 weeks, about 2.5 weeks, about 3 weeks, about 3.5 weeks, or about 4 weeks, prior to, and/or after a stressor.

In certain embodiments, the administration of the 5-HT₄R agonist, and ketamine, ketamine analog, or a pharmaceutically acceptable salt, derivative, or metabolite thereof, NMDAR antagonist or AMPAR agonist or the composition or compositions comprising them is continued over a period of up to 2 days, up to 3 days, up to 4 days, up to 5 days, up to 6 days, up to 1 week, up to 2 weeks, up to 3 weeks, up to 4 weeks, about 2 days, about 3 days, about 4 days, about 5 days, about 6 days, about 1 week, about 2 weeks, about 3 weeks, about 4 weeks, about 5 weeks, about 6 weeks, about 7 weeks, about 8 weeks, about 9 weeks, about 10 weeks, or longer.

In certain embodiments, the 5-HT₄R agonist, and ketamine, ketamine analog, or a pharmaceutically acceptable salt, derivative, or metabolite thereof, NMDAR antagonist or AMPAR agonist or the composition or compositions comprising them is administered once, twice, at least twice, at least three times, at least four times, at least five time, at least six times, at least seven times, at least eight times, at least nine times, or more per treatment.

In certain embodiments, the 5-HT₄R agonist, and ketamine, ketamine analog, or a pharmaceutically acceptable salt, derivative, or metabolite thereof, NMDAR antagonist or AMPAR agonist or the composition or compositions comprising them is administered at least once a day, at least twice a day, at least three times per day, at least once a week, at least twice a week, at least three times a week, at least once per month, at least twice per month, or more frequently. Treatment can continue as long as needed.

In certain embodiments, the 5-HT₄R agonist, and ketamine, ketamine analog, or a pharmaceutically acceptable salt, derivative, or metabolite thereof, NMDAR antagonist or AMPAR agonist or the composition or compositions comprising them is administered at least once before a stressor and at least once after a stressor.

The 5-HT₄R agonist, and ketamine, ketamine analog, or a pharmaceutically acceptable salt, derivative, or metabolite thereof, NMDAR antagonist or AMPAR agonist or the composition or compositions comprising them may be administered daily, weekly, biweekly, several times daily, semi-weekly, every other day, bi-weekly, quarterly, several times per week, semi-weekly, monthly etc. The duration and frequency of treatment may depend upon the subject’s response to treatment.

### Stressors

A stressor is a stimulus that causes stress. It can be an event or other factor that disrupts the body’s homeostasis of temperature, blood pressure, and/or other functions. In certain embodiments, a stressor is a traumatic or stressful event. Because humans have sophisticated brains and thought processes, anticipating a disruption can also be a stressor. In certain embodiments, a stressor is injury, trauma, combat, warfare, surgery, an accident, a criminal assault, child abuse, natural or human-caused disasters, a crash, grief, hunger, heat, cold, chemical exposure, autoimmune disease, infectious disease, viral infection, cancer, exhaustion, physical distress, neuropathy, hyperalgesia, allodynia, emotional distress, or depression. A traumatic event may be an event or something that threatens the person’s life or the life of a close one or it could be something witnessed. U.S. Pat. Application No. 20140018339.

A stressor may be acute or may be chronic.

There are numerous physiological processes that are altered in response to stress. Among these are altered cortisol, corticotropin, catecholamine and serotonin levels. These levels return to baseline after an acute stressor is removed. These biochemical markers of stress in turn lead to ill health and psychosocial disorders. Consequently, stress plays a major role in physical and mental health. Stress can affect the onset of, or susceptibility to disease. It can also affect the progression or course of disease even when there is another underlying pathophysiology of the disease. Recovery from an existing disease can also be delayed due to stress. For example, stress is a contributing factor to high blood pressure, heart disease, headaches, colitis, irritable bowel syndrome, temporo-mandibular joint disorder, cancer, peptic ulcers, insomnia, skin disorders and asthma. Stress can also aggravate other conditions such as multiple sclerosis, diabetes, herpes, mental illness, substance abuse and psychiatric disorders characterized by the presence of violent or aggressive tendencies. Particularly, stress contributes to functional somatic disorders, affective disorders and major depressive disorder (MDD). These include disorders such as chronic fatigue syndrome (CFS), fibromyalgia (FMS), Gulf War Syndrome, anxiety and post-traumatic stress disorder (PTSD). Stressors that disrupt normal exercise or sleep patterns.

Additional examples of use include administration prior to military deployment to protect Service members (active combat soldiers, battlefield surgeons, etc.) and even military working dogs against stress. Potential non-military use cases include, but are not limited to: police, firefighters, first responders, emergency medical technicians (EMTs), emergency room (ER) doctors, prison guards (and prisoners), humanitarian aid workers, and refugees.

In certain embodiments, a subject may be administered the present agents, compounds, composition or compositions prior to a situation in which the subject (such as an early responder or military personnel) is likely to be exposed to traumatic stress, immediately after exposure to traumatic stress, and/or when the subject feels that his or her PTSD symptoms are likely to appear.

### Resilience to Stress

Resilience to stress refers to the capacity of a subject to adapt or change successfully, and/or to maintain physiological, neurological, or psychological homeostasis, in the face of a stressor (e.g., adversity). As used herein, the term “enhancing resilience” refers to increasing the ability of a subject to experience a stressor (e.g., a traumatic event) without suffering a stress-induced affective disorder, and/or with less post-event symptomatology or disruption of homeostasis and/or normal activities of daily living. In certain embodiments, improving resilience can prevent a stress-induced affective disorder. In certain embodiments, improving resilience can reduce at least one of the signs, symptoms, or symptom clusters of a stress-induced affective disorder. In certain embodiments, the present method enhances a subject’s resilience to stress, helps protect against developing stressor-related psychopathology, decrease the functional consequences of stressor-induced disorders (e.g., PTSD, etc.), and reduce medical morbidity and mortality.

The Connor-Davidson Resilience Scale (CD-RISC) is a 25-item self-report scale, each rated on a 5-point scale (0-4), with higher scores reflecting greater resilience (Connor K M & Davidson, J R T. Development of a new resilience scale: the Connor-Davidson Resilience Scale (CD-RISC). Depression and Anxiety, 2003: 18:71-82).

Resilience, psychological growth and life satisfaction may be measured with the CD-RISC, the Purpose in Life Scale, the abbreviated MOS Social Support Survey, the PTGI, and the Q-LES-Q.

### Stress-Induced Affective Disorders

There are numerous disorders that are either caused by or exacerbated by stress. The present compositions and methods can prevent or delay a stress-induced affective disorder or stress-induced psychopathology. Stress-induced affective disorders or stress-induced psychopathologies which may be prevented or treated by the present compositions and methods include, but are not limited to: addictive disorders such as substance abuse, anorexia, bulimia, obesity, smoking addiction, and weight addiction; anxiety disorders such as agoraphobia, anxiety disorder, obsessive compulsive disorder, panic attacks, performance anxiety, phobias, and post-traumatic stress disorder (PTSD); psychiatric disorders such as stress-induced psychiatric disorders; autoimmune diseases such as allergies, arthritis, fibromyalgia, fibromytosis, lupus, multiple sclerosis, rheumatoid arthritis, Sjogren’s syndrome, and vitiligo; cancer such as bone cancer, brain cancer, breast cancer, cervical cancer, colon cancer, Hodgkin’s disease, leukemia, liver cancer, lung cancer, lymphoma, multiple myeloma, ovarian cancer, pancreatic cancer, and prostate cancer; cardiovascular disorders such as arrhythmia, arteriosclerosis, Burger’s disease, essential hypertension, fibrillation, mitral valve prolapse, palpitations, peripheral vascular disease, Raynaud’s disease, stroke, tachycardia, and Wolff-Parkinson-White Syndrome; and developmental disorders such as attention deficit disorder, concentration problems, conduct disorder, dyslexia, hyperkinesis, language and speech disorders, and learning disabilities.

### Anxiety Disorders

The compositions and methods can prevent or delay an anxiety disorder. The five major types of anxiety disorders are: panic disorder; obsessive-compulsive disorder; post-traumatic stress disorder; generalized anxiety disorder; and phobias (including social phobia, also called social anxiety disorder). Each anxiety disorder has its own distinct features, but they are all bound together by the common theme of excessive, irrational fear and dread. It is common for an anxiety disorder to accompany depression, eating disorders, substance abuse, or another anxiety disorder.

Panic disorder is characterized by repeated episodes of intense fear that strike often and without warning. Physical symptoms include chest pain, heart palpitations, shortness of breath, dizziness, abdominal distress, feelings of unreality, and fear of dying. Obsessive-compulsive disorder is characterized by repeated, unwanted thoughts or compulsive behaviors that seem impossible to stop or control. Generalized Anxiety Disorder is characterized by exaggerated worrisome thoughts and tension about everyday routine life events and activities, lasting at least six months. Almost always anticipating the worst even though there is little reason to expect it; accompanied by physical symptoms, such as fatigue, trembling, muscle tension, headache, or nausea. Phobias are characterized into two major types of phobias, social phobia and specific phobia. People with social phobia have an overwhelming and disabling fear of scrutiny, embarrassment, or humiliation in social situations, which leads to avoidance of many potentially pleasurable and meaningful activities. People with specific phobia experience extreme, disabling, and irrational fear of something that poses little or no actual danger; the fear leads to avoidance of objects or situations and can cause people to limit their lives unnecessarily.

### Posttraumatic Stress Disorder (PTSD)

Typically, a subject suffering from PTSD was exposed to a traumatic event in which the person experienced, witnessed, or was confronted with an event or events that involved actual or threatened death or serious injury, or a threat to the physical integrity of self or others and the person’s response involved intense fear, helplessness, or horror.

Having repeated intrusive memories of the trauma exposure is one of the core symptoms of PTSD. Patients with PTSD are known to display impairments in learning and memory during neuropsychological testing. Other core symptoms of PTSD include heightened stress sensitivity (startle), tension and anxiety, memory disturbances, and dissociation.

In certain embodiments, the present compositions and methods prevent or inhibit the development of PTSD in a subject. In certain embodiments, the present compositions and methods prevent or inhibit the development of one or more PTSD-like symptoms. In certain embodiments, a subject may be administered the present agents, compounds or compositions prior to a situation in which the subject (such as an early responder or military personnel) is likely to be exposed to traumatic stress, immediately after exposure to traumatic stress, and/or when the subject feels that his or her PTSD symptoms are likely to appear.

Typically, the traumatic event is persistently re-experienced in one or more of the following ways: recurrent and intrusive distressing recollections of the event, including images, thoughts, or perceptions; recurrent distressing dreams of the event; acting or feeling as if the traumatic event were recurring (includes a sense of reliving the experience, illusions, hallucinations, and dissociative flashback episodes, including those that occur on awakening or when intoxicated); intense psychological distress at exposure to internal or external cues that symbolize or resemble an aspect of the traumatic event; and physiological reactivity on exposure to internal or external cues that symbolize or resemble an aspect of the traumatic event. An individual suffering from PTSD also has persistent avoidance of stimuli associated with the trauma and numbing of general responsiveness (not present before the trauma), as indicated by 3 or more of the following: efforts to avoid thoughts, feelings, or conversations associated with the trauma; efforts to avoid activities, places, or people that arouse recollections of the trauma; inability to recall an important aspect of the trauma; significantly diminished interest or participation in significant activities; feeling of detachment or estrangement from others; restricted range of affect (e.g., unable to have loving feelings); sense of a foreshortened future (e.g., does not expect to have a career, marriage, children, or a normal life span); and persistent symptoms of increased arousal (not present before the trauma); or as indicated by 2 or more of the following: difficulty falling or staying asleep; irritability or outbursts of anger; difficulty concentrating; hypervigilance; and exaggerated startle response. The disturbance, which has lasted for at least a month, causes clinically significant distress or impairment in social, occupational, or other important areas of functioning.

In certain embodiments, the present compositions and methods prevent, reduce, eliminate or delay one or more of the symptoms including, but not limited to: re-experiencing of the traumatic experience in the form of intrusive memories, nightmares, or flashbacks; emotional and physical reactions triggered by reminders of the trauma; distancing from others; decreased interest in activities and other people; numbing of feelings; avoidance of trauma reminders; hyperarousal symptoms, including disrupted sleep, irritability, hypervigilance, decreased concentration; increased startle reflex; and combinations thereof.

Whatever the source of the problem, some people with PTSD repeatedly relive the trauma in the form of nightmares and disturbing recollections during the day. They may also experience other sleep problems, feel detached or numb, or be easily startled. They may lose interest in things they used to enjoy and have trouble feeling affectionate. They may feel irritable, more aggressive than before, or even violent. Things that remind them of the trauma may be very distressing, which could lead them to avoid certain places or situations that bring back those memories.

The disorder may be accompanied by depression, substance abuse, or one or more other anxiety disorders. In severe cases, the person may have trouble working or socializing.

### Major Depressive Disorder

Major depressive disorder refers to a class of syndromes characterized by negative affect and repeated episodes of depression without any history of independent episodes of mood elevation and over-activity that fulfill the criteria of mania. The age of onset and the severity, duration and frequency of the episodes of depression are all highly variable. The disorder may begin at any age. The symptoms of major depressive disorder typically develop over days to weeks. Prodromal symptoms include generalized anxiety, panic attacks, phobias or depressive symptoms and may occur during several months preceding the episode. Individual episodes also last between 3 and 12 months but recur less frequently. Most patients are asymptomatic between episodes, but a minority of patients may develop a persistent depression, mainly in old age. Individual episodes of any severity are often precipitated by stressful life events. Common symptoms of a depressive episode include: reduced concentration and attention; reduced self-esteem and self-confidence; ideas of guilt and unworthiness, ideas or acts of self-harm or suicide; disturbed sleep; and diminished appetite. In certain embodiments, a major depressive episode follows a psychosocial stressor, e.g., death of a loved one, marital separation, childbirth or the end of an important relationship.

The lowered mood varies little from day to day and is often unresponsive to circumstances, yet may show a characteristic diurnal variation as the day goes on. As with manic episodes, the clinical presentation shows marked individual variations, and atypical presentations are particularly common in adolescence. In some cases, anxiety, distress, and motor agitation may be more prominent at times that the depression, and the mood change may also be masked by added features such as irritability, excessive consumption of alcohol, histrionic behavior, and exacerbation of pre-existing phobic or obsessional symptoms, or by hypochondria.

### Combination Therapy

The present agents, compounds, composition or compositions may be administered to a subject alone, or may be administered to a subject in combination with one or more additional agents.

In certain embodiments, the additional agent is an anti-depressant, an anxiolytic, or combinations thereof. In certain embodiments, the additional agent is a serotonin reuptake inhibitor (SRI), or a selective serotonin reuptake inhibitor (SSRI). In certain embodiments, the additional agent is fluoxetine, paroxetine, sertraline, lithium, riluzole, prazosin, lamotrigine, ifenprodil, or combinations thereof. In certain embodiments, the additional agent is a dual serotonin norepinephrine reuptake inhibitor compound (DRI). In certain embodiments, the additional agent is venlafaxine, duloxetine, milnacipran, or combinations thereof. In certain embodiments, the additional agent is a non-tricyclic triple reuptake inhibitor (TRI).

In certain embodiments, the present agents, compounds, composition or compositions are administered to a subject in combination with one or more additional agents such as antidepressants, analgesics, muscle relaxants, anorectics, stimulants, antiepileptic drugs, and sedative/hypnotics. Non-limiting examples of additional agents that can be administered in combination with the present agents, compounds, composition or composition include, but are not limited to, neurontin, pregabalin, pramipexole, L-DOPA, amphetamine, tizanidine, clonidine, tramadol, morphine, tricyclic antidepressants, codeine, carbamazepine, sibutramine, amphetamine, valium, trazodone and combinations thereof.

In certain embodiments, combination therapy means simultaneous administration of the agents in the same dosage form, simultaneous administration in separate dosage forms, or separate administration of the agents.

In certain embodiments, the additional agent is used as adjunctive therapy to the present agents, compounds, composition or compositions. In certain embodiments, the treatment includes a phase wherein treatment with the additional agent takes place after treatment with the present agents, compounds, composition or compositions has ceased. In certain embodiments, the treatment includes a phase where treatment with the present agents, compound, composition or compositions and treatment with the additional agent/treatment overlap.

Combination therapy can be sequential or can be administered simultaneously. In either case, these agents, compounds and compositions are said to be “co-administered.” It is to be understood that “co-administered” does not necessarily mean that the agents, compounds and compositions are administered in a combined form (i.e., they may be administered separately (e.g., as separate compositions or formulations) or together (e.g., in the same formulation or composition) to the same or different sites at the same or different times).

In certain embodiments, a subject is treated concurrently (or concomitantly) with the present agents, compounds, composition or compositions and an additional agent. In certain embodiments, a subject is treated initially with the present agents, compounds, composition or compositions, followed by cessation of the present agents, compounds, composition or compositions and initiation of treatment with an additional agent. In certain embodiments, the present agents, compounds, composition or compositions are used as an initial treatment, e.g., by administration of one, two or three doses, and an additional agent is administered to prolong the effect of the present agents, compounds, composition or compositions, or alternatively, to boost the effect of the present agents, compounds, composition or compositions. A person of ordinary skill in the art will recognize that other variations of the presented schemes are possible, e.g., initiating treatment of a subject with the present agents, compounds, composition or compositions, followed by a period wherein the subject is treated with an additional agent as adjunct therapy to the present agents, compounds, composition or compositions treatment, followed by cessation of the present agents, compounds, composition or compositions treatment.

The present agents, compounds, composition or compositions and the additional pharmaceutically active agent(s) may be administered together or separately and, when administered separately this may occur simultaneously or sequentially in any order. The amounts of the present agents, compounds, composition or compositions and the additional pharmaceutically active agent(s) and the relative timings of administration will be selected in order to achieve the desired combined therapeutic effect.

In various embodiments, the therapies (e.g., agents, compounds, composition or compositions provided herein and an additional agent in a combination therapy) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In certain embodiments, the therapies are administered no more than 24 hours apart or no more than 48 hours apart. In certain embodiments, two or more therapies are administered within the same patient visit. In other embodiments, the agents, compounds, composition or compositions provided herein and the additional agent are administered concurrently. In other embodiments, the agents, compounds, composition or compositions provided herein and the additional agent are administered at about 2 to 4 days apart, at about 4 to 6 days apart, at about 1 week part, at about 1 to 2 weeks apart, or more than 2 weeks apart. In certain embodiments, administration of the same agent may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same agent may be repeated and the administration may be separated by at least at least 1 day, 2 days, 3 days, 5 days, 10 days, 15 days, 30 days, 45 days, 2 months, 75 days, 3 months, or 6 months.

In certain embodiments, the agents, compounds, composition or compositions provided herein and a second agent are administered to a subject in a sequence and within a time interval such that the agents, compounds, composition or compositions provided herein can act together with the additional agent to provide an increased benefit than if they were administered otherwise. In one embodiment, the agents, compounds, composition or compositions provided herein and the additional agent exerts their effect at times which overlap. Each additional agent can be administered separately, in any appropriate form and by any suitable route. In other embodiments, the agents, compounds, composition or compositions provided herein is administered before, concurrently or after administration of the second active agent. The additional agent can act additively or synergistically with the agents, compounds, composition or compositions provided herein.

In one embodiment, the agents, compounds, composition or compositions provided herein is administered concurrently with one or more second agents in the same pharmaceutical composition. In another embodiment, a agents, compounds, composition or compositions composition provided herein is administered concurrently with one or more additional agents in separate pharmaceutical compositions.

Also contemplated are administration of agents, compounds, composition or compositions provided herein and an additional agent by the same or different routes of administration, e.g., oral and parenteral.

### Kits

Also provided are kits for use in the present methods of prophylactically treating a stress-induced affective disorder.

The kits can include agents, compounds, composition or compositions provided herein, and instructions providing information to a health care provider regarding usage in accordance with the present methods. The kit may optionally contain an additional agent or composition. Instructions may be provided in printed form or in the form of an electronic medium such as a floppy disc, CD, or DVD, or in the form of a website address where such instructions may be obtained. A unit dose of agents, compounds, composition or compositions provided herein can include a dosage such that when administered to a subject, a therapeutically or prophylactically effective plasma level of the compound or composition can be maintained in the subject for at least 1 days. In some embodiments, a compound or composition can be included as a sterile aqueous pharmaceutical composition or dry powder (e.g., lyophilized) composition. In some embodiments, suitable packaging is provided. As used herein, “packaging” includes a solid matrix or material customarily used in a system and capable of holding within fixed limits a compound provided herein and/or a second agent suitable for administration to a subject. Such materials include glass and plastic (e.g., polyethylene, polypropylene, and polycarbonate) bottles, vials, paper, plastic, and plastic-foil laminated envelopes and the like.

The kits described herein contain one or more containers, which contain compounds, signaling entities, biomolecules and/or particles as described. The kits also contain instructions for mixing, diluting, and/or administrating the compounds. The kits also include other containers with one or more solvents, surfactants, preservative and/or diluents (e.g., saline (0.9% NaCl), or 5% dextrose) as well as containers for mixing, diluting or administering the components to the sample or to the patient in need of such treatment.

The compositions of the kit may be provided as any suitable form, for example, as liquid solutions or as dried powders. When the composition provided is a dry powder, the powder may be reconstituted by the addition of a suitable solvent, which may also be provided. In embodiments where liquid forms of the composition are used, the liquid form may be concentrated or ready to use. The solvent will depend on the compound and the mode of use or administration. Suitable solvents for drug compositions are well known and are available in the literature. The solvent will depend on the compound and the mode of use or administration.

The kits comprise a carrier being compartmentalized to receive in close confinement one or more container such as vials, tubes, and the like, each of the container comprising one of the separate elements to be used in the method. For example, one of the container may comprise a positive control in an assay. Additionally, the kit may include containers for other components, for example, buffers useful in the assay.

### EXAMPLES

The present invention may be better understood by reference to the following non-limiting examples, which are presented in order to more fully illustrate the preferred embodiments of the invention. They should in no way be construed to limit the broad scope of the invention.

### Example 1 - Materials and Methods for Examples 2-7

### Mice

Male and female 129S6/SvEvTac mice were purchased from Taconic (Hudson, NY) at 7 weeks of age. Female C57BL/6NTac mice were purchased from Taconic (Hudson, NY) at 7 weeks of age. Mice were housed 5 per cage in a 12-h (06:00-18:00) light-dark colony room at 22° C. All experiments were approved by the Institutional Animal Care and Use Committee (IACUC) at the New York Psychiatric Institute (NYSPI).

### Drugs

A single injection of saline (0.9% NaCl), or (R,S)-ketamine (Fort Dodge Animal Health, Fort Dodge, IA) (10 mg/kg or 30 mg/kg), and/or prucalopride (SML1371, Sigma-Aldrich, St. Lois, MO) (1.5 mg/kg or 3 mg/kg), was administered once during the course of each experiment at approximately 6-8 months of age. All drugs were prepared in physiological saline and administered intraperitoneally (i.p.) in volumes of 0.1 cc per 10 mg body weight.

### Behavioral Assays

For all experiments, food and water were provided ad libitum, unless otherwise noted. Behavioral testing was performed during the light phase.

### Contextual Fear Conditioning (CFC)

A 3-shock CFC paradigm was administered as previously described (Denny et al., 2014; Drew et al., 2010). Fear conditioning was conducted in chambers obtained from Coulborn Instruments (Allentown, PA) with internal dimensions of approximately 20 cm wide × 16 cm deep × 20.5 cm high. The chambers had metal walls on each side, clear plastic front and back walls and ceilings, and stainless-steel bars on the floor. A house light (CM1820 bulb, 28 V, 100 mA) mounted directly above the chamber provided illumination. Each chamber was located inside a larger, insulated, plastic cabinet that provided protection from outside light and noise. Each cabinet contained a ventilation fan that was operated during the sessions. A paper towel dabbed with lemon solution was placed underneath the chamber floor. Mice were held outside the experimental room in their home cages prior to testing and transported to the conditioning apparatus individually in standard mouse cages. Chambers were cleaned with 70% EtOH between each set of mice. Training sessions were conducted using a 3-shock protocol. Mice were placed into the conditioning chamber and received shocks at 180 s, 240 s, and 300 s (2 s duration each, 0.75 mA). Fifteen seconds after the last shock, mice were removed from the chamber. Overall, the training session lasted 317 s. During re-exposure, mice were placed in the conditioning chamber for 5 minutes and did not receive any shocks. All sessions were scored for freezing using FreezeFrame4 (Actimetrics, Wilmette, IL).

### Forced Swim Test (FST)

The FST is typically used in rodents to screen for potential human antidepressants. In fact, many papers examining ketamine in mouse models only observe effects in the FST. In the FST, time spent immobile, as opposed to swimming, is used as a measure of depressive behavior.

The FST was administered as previously described (Brachman et al., 2016). Briefly, mice were placed into clear plastic buckets 20 cm in diameter and 23 cm deep filled ⅔ of the way with 22° C. water. Mice were videotaped from the side for 6 minutes and were exposed to the swim test on 2 consecutive days. Immobility time was scored by an experimenter blind to the experimental groups.

### Elevated Plus Maze (EPM)

Testing was performed as previously described (Saxe et al., 2006). Briefly, the maze is a plus-cross-shaped apparatus consisting of four arms, two open and two enclosed by walls, linked by a central platform at a height of 50 cm from the floor. Mice were individually placed in the center of the maze facing an open arm and were allowed to explore the maze for 5 minutes. The time spent in and the number of entries into the open arms was used as an anxiety index. Videos were scored using ANY-maze behavior tracking software (Stoelting, Wood Dale, IL).

### Open Field (OF)

The OF assay was administered as previously described (David et al., 2009). Briefly, motor activity was quantified in four Plexiglas open field boxes 43 ×43 cm² (MED Associates, St. Albans, VT). Two sets of 16 pulse-modulated infrared photobeams on opposite walls 2.5-cm apart recorded x-y ambulatory movements. Activity chambers were computer interfaced for data sampling at 100-ms resolution. The computer defined grid lines that dividing center and surround regions, with the center square consisting of four lines 11 cm from the wall.

### Novelty Suppressed Feeding (NSF)

Testing was performed as previously described (Brachman et al., 2016). Briefly, the testing apparatus consisted of a plastic box (50 × 50 × 20 cm). The floor was covered with approximately 2 cm of wooden bedding and the arena was brightly lit (1100-1200 lux). For 129S6/SvEv experiments, mice were food restricted for 12 h. All food was removed from the home cage. At the time of testing, a single pellet of food (regular chow) was placed on a white paper platform positioned in the center of the box. Each animal was placed in a corner of the box, and a stopwatch was immediately started. The latency of the mice to begin eating was recorded. Immediately after the latency was recorded, the food pellet was removed from the arena. The mice were then placed into their home cage and the amount of food consumed in 5 min was measured (home cage consumption), followed by an assessment of post-restriction weight. Kaplan-Meier survival analysis was used due to the lack of normal distribution of data. The Mantel-Cox log-rank test was used to evaluate differences between the experimental groups.

### Marble Burying (MB)

The MB assay was conducted in a clean cage (10.5 in × 5.5 in) containing soft pliable Beta Chip bedding (Northeastern Products Corp, Warrensburg, NY). The cage contained 16 marbles set up in 4 rows of 4 across. Mice were given 30 minutes to explore and bury. At the end of the assay, the percentage of marbles buried was calculated.

### Statistical Analysis

All data were analyzed using StatView 5.0 (SAS Institute, Cary, NC) or Prism 7.0 (Graphpad Software, La Jolla, CA). Alpha was set to 0.05 for all analyses. Generally, the effect of Drug or Group was analyzed using an analysis of variance (ANOVA), using repeated measures where appropriate. Post-hoc Dunnett, Sidak, or Tukey tests were used where appropriate.

### Example 2 - Combined Prophylactic (R,S)-Ketamine and Prucalopride Decreased Fear, Depressive-Like Behavior and Anxiety-Like Behavior, i.e., Protects Against Stress in Male Mice

In a recent study, the inventors reported that three 5HT₄R agonists are effective as prophylactics against stress (Chen et al., 2020). Prucalopride attenuated learned fear and decreased stress-induced depressive-like behavior but had no prophylactic effect on stress-induced anxiety-like behavior. Electrophysiological recordings following (R,S)-ketamine or prucalopride administration revealed that both drugs alter AMPA receptor-mediated synaptic transmission in CA3 even though their primary targets are NDMARs and 5-HT₄Rs, respectively. Here, it was hypothesized that combined administration of (R,S)-ketamine and prucalopride may result in additive effects or be effective as prophylactics at a lower dose.

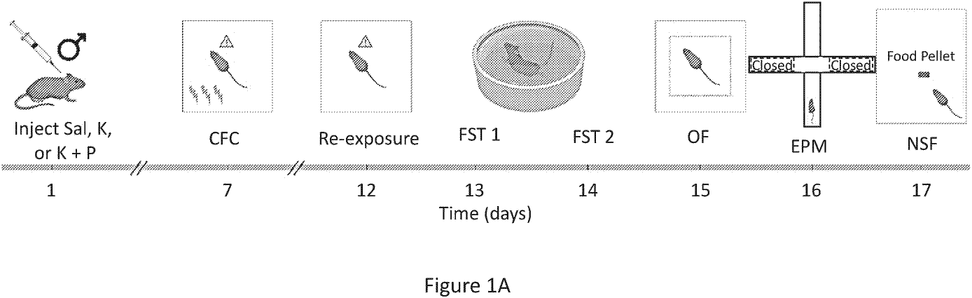

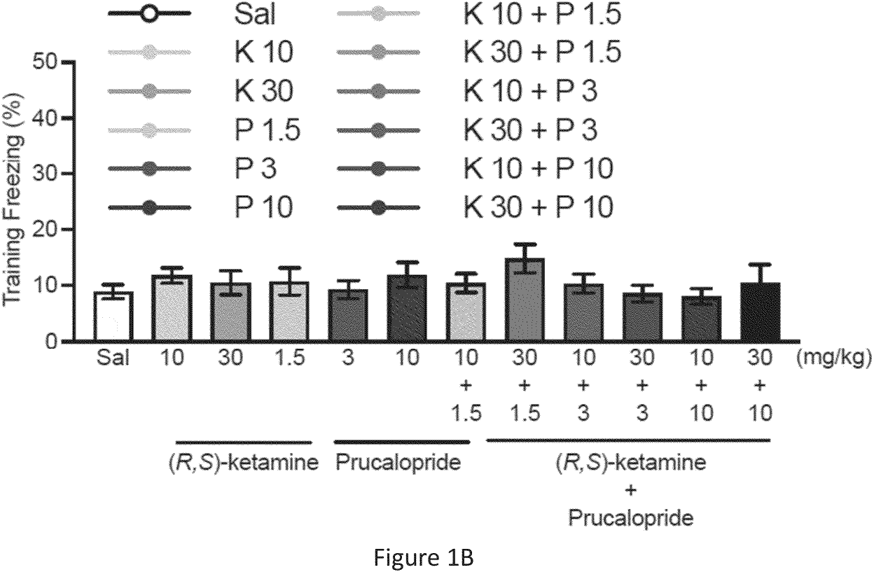

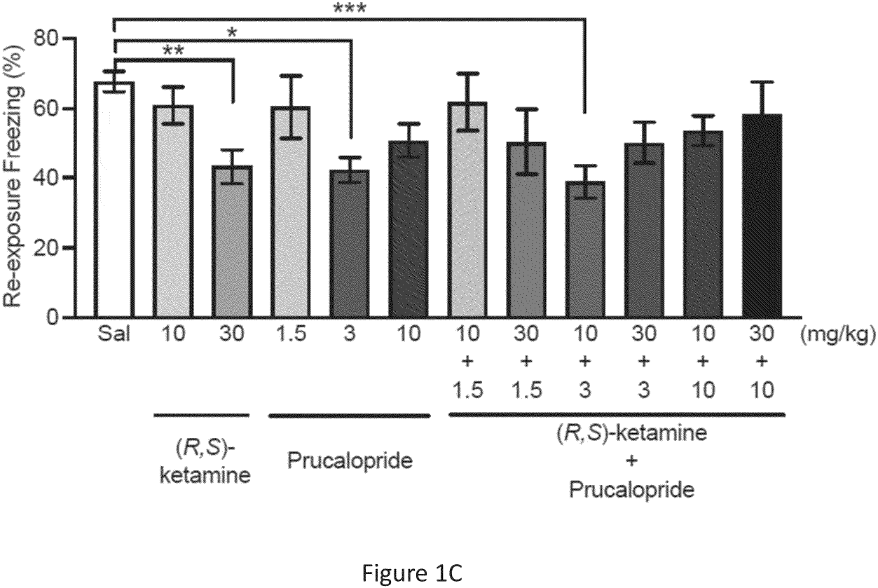

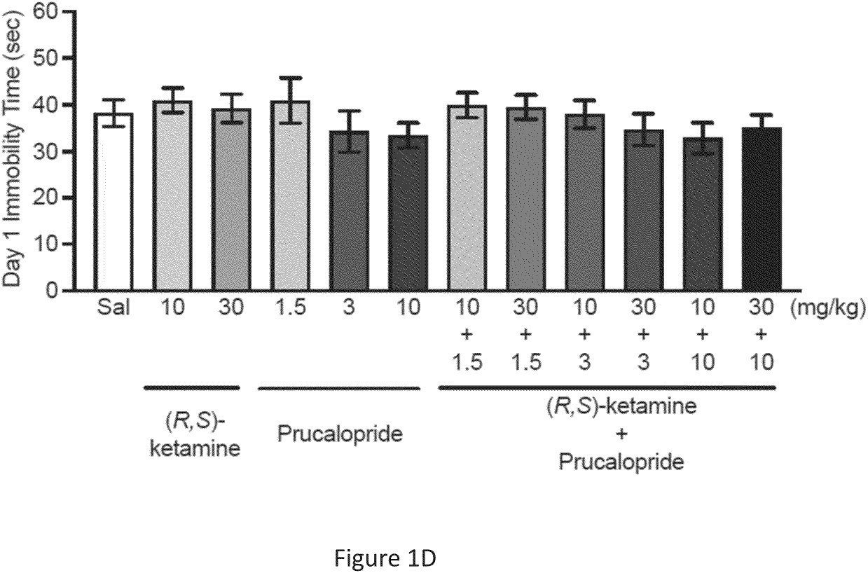

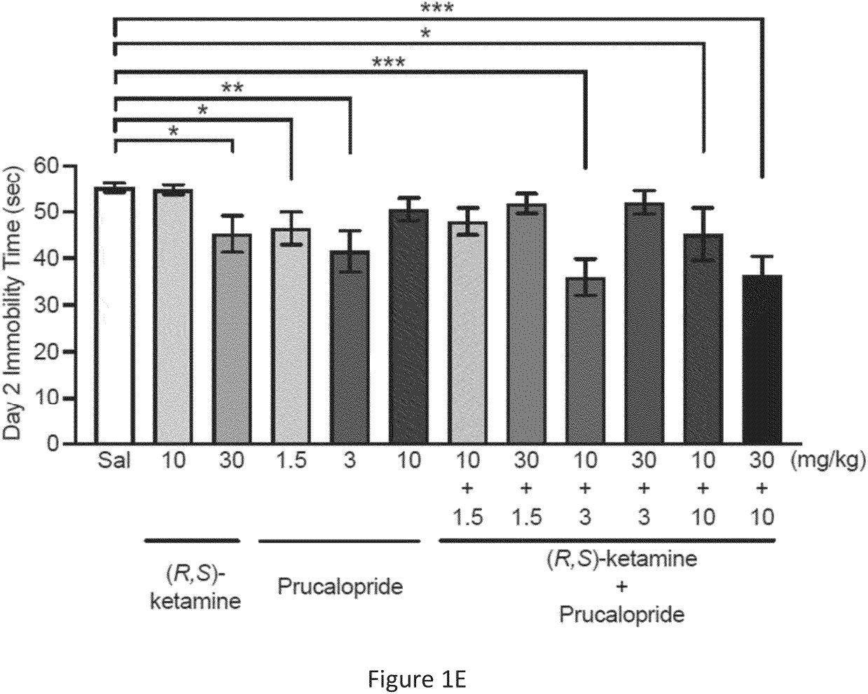

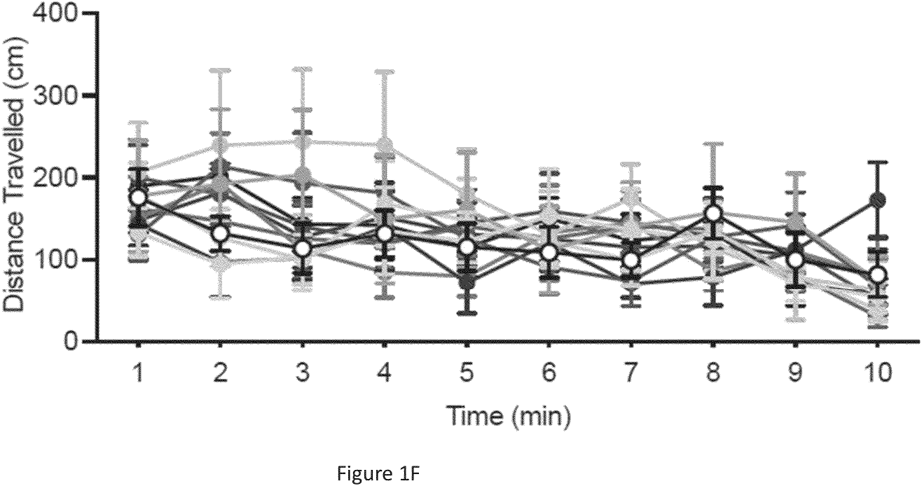

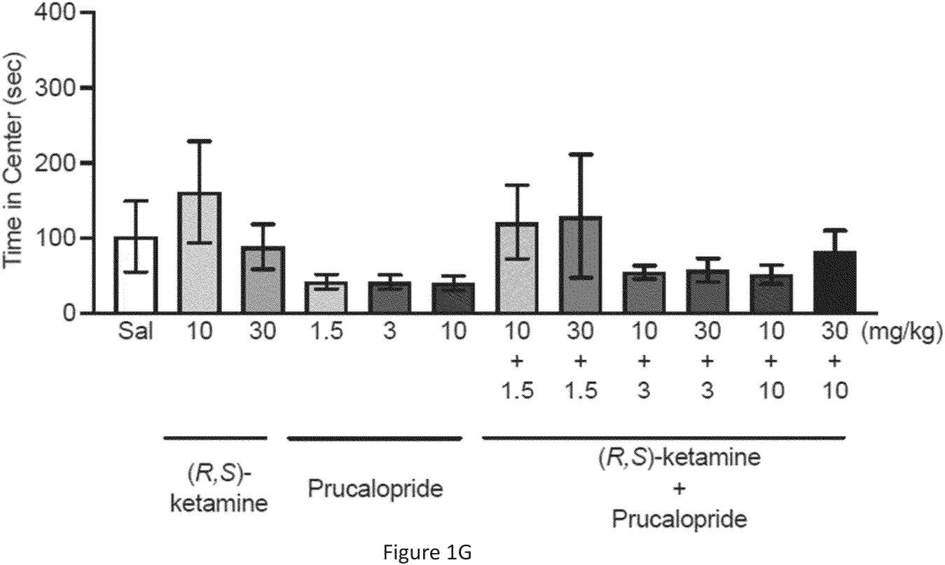

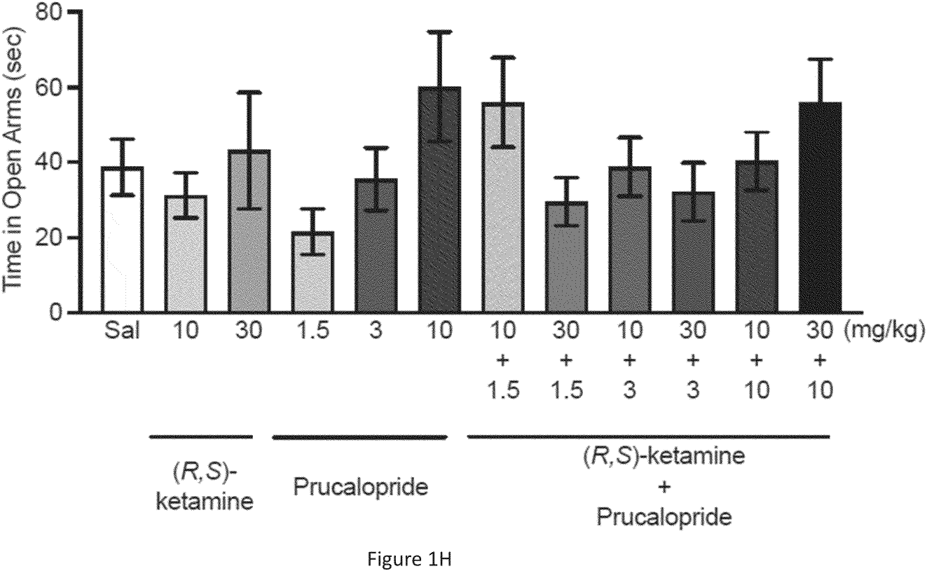

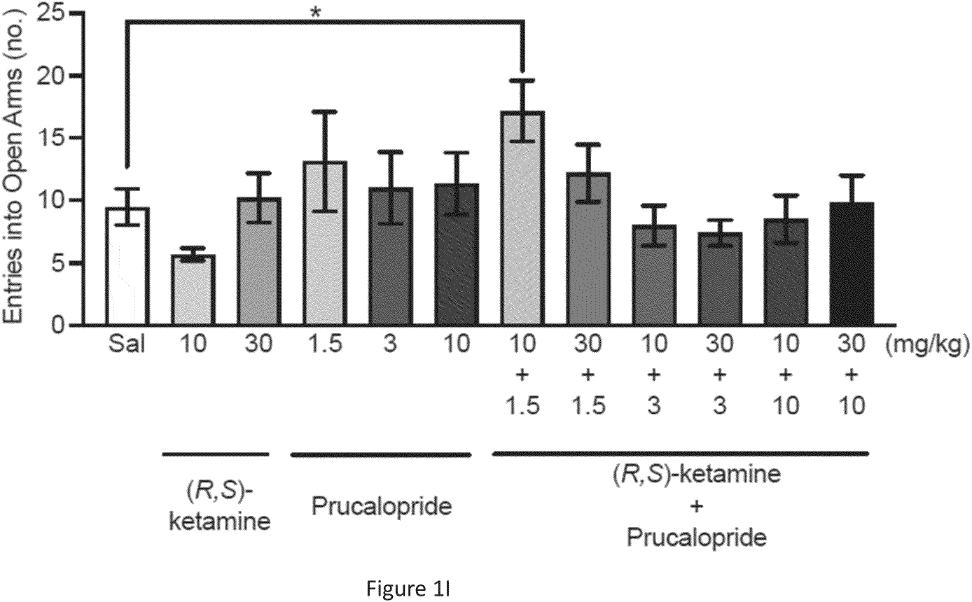

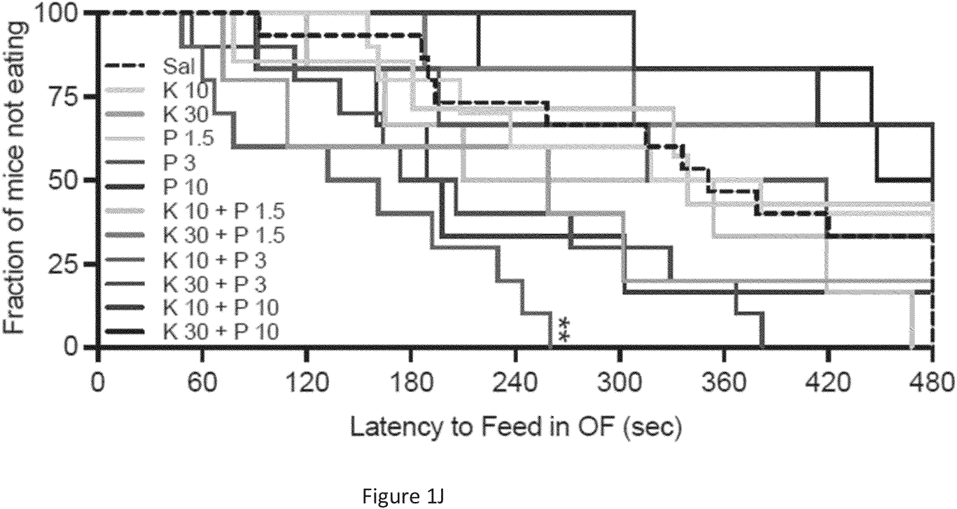

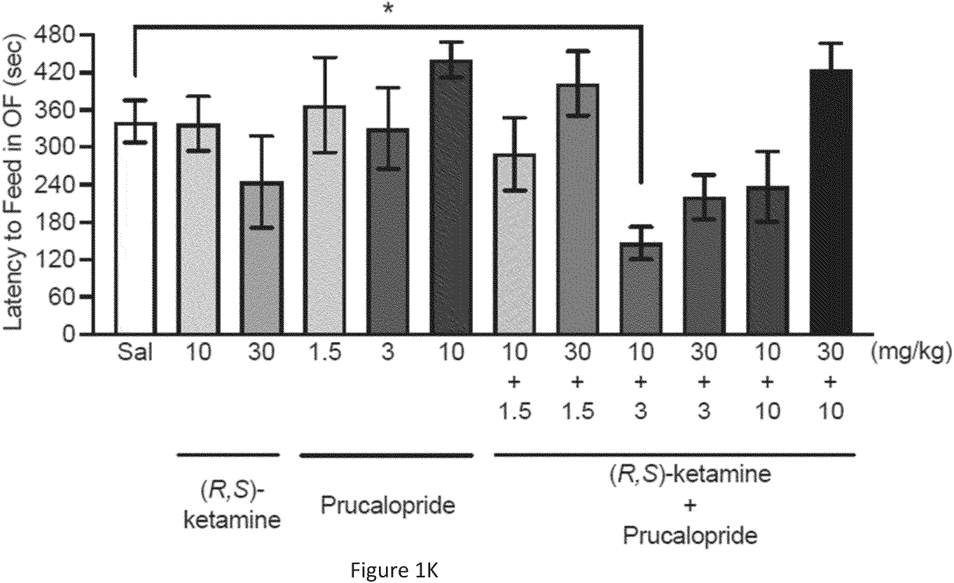

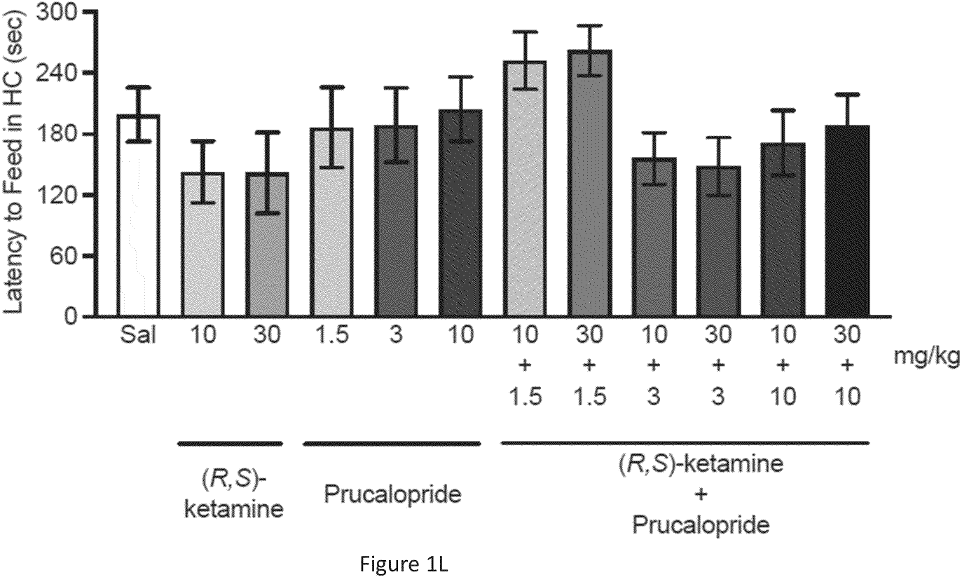

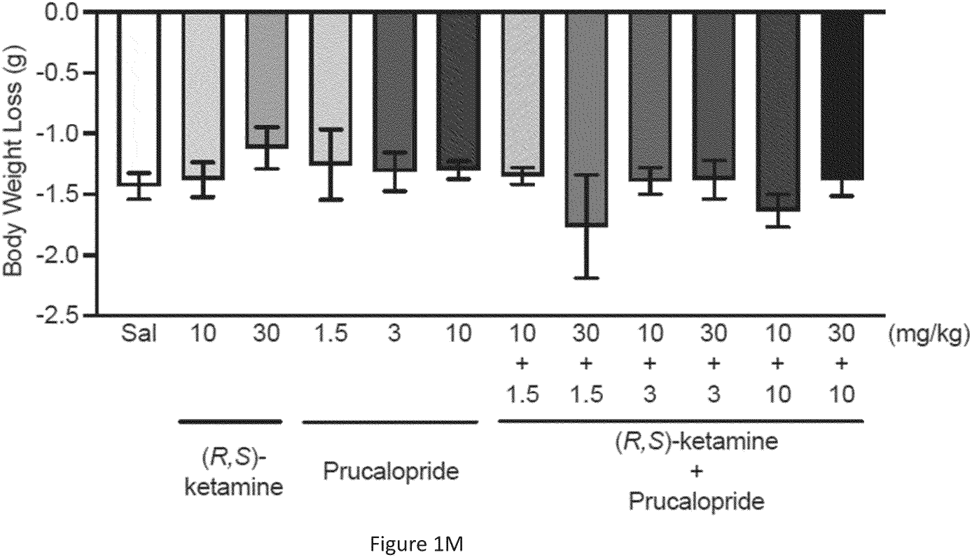

Male mice were injected with saline, (R,S)-ketamine, prucalopride, or combined (R,S)-ketamine and prucalopride. Five days later mice were administered a 3-shock CFC paradigm followed by behavior testing for fear, depressive-, and anxiety-like behavior using the forced swim test (FST), open field (OF), elevated plus maze (EPM), and novelty-suppressed feeding (NSF). See FIG. 1A.

All groups had comparable freezing during CFC training (FIG. 1B). However, during re-exposure to the CFC context (R,S)-ketamine (30 mg/kg), prucalopride (3 mg/kg), and (R,S)-ketamine and prucalopride (10 + 3 mg/kg) were effective at decreasing fear when compared with saline (FIG. 1C). All other groups had comparable freezing levels.

During day 1 of the FST, all groups had comparable immobility time (FIG. 1D). Conversely, during day 2 of the FST, (R,S)-ketamine (30 mg/kg), prucalopride (1.5 and 3 mg/kg), and (R,S)-ketamine and prucalopride (10 + 3 mg/kg; 10 + 10 mg/kg; 30 + 10 mg/kg) were effective at decreasing fear when compared with saline (FIG. 1E). Interestingly, the combined dose of (R,S)-ketamine and prucalopride (10 + 10 mg/kg) was effective at decreasing immobility time, but not when administered separately at those doses.

All groups traveled a comparable distance during the OF (FIG. 1F) and spent a comparable amount of time in the center of the OF (FIG. 1G) as well as in the open arms of the EPM (FIG. 1H). However, (R,S)-ketamine and prucalopride (10 + 1.5 mg/kg) increased the number of entries into the open arms of the EPM (FIG. 1I).

In the NSF paradigm, prior work indicated that (R,S)-ketamine and prucalopride were not effective at reducing hyponeophagia. However, when (R,S)-ketamine and prucalopride were combined at 10 + 3 mg/kg, respectively, the latency to feed in the OF was significantly decreased when compared with saline (FIGS. 1J and 1K). The latency to eat in the home cage (FIG. 1L) and the weight loss (%) (FIG. 1M) did not differ between the groups (results not shown).

### Example 3 - Combined Prophylactic (R,S)-Ketamine and Prucalopride Decreased Fear, Depressive-Like Behavior and Anxiety-Like Behavior, i.e., Protects Against Stress, in Female Mice

In a recent study, the inventors also reported RS-67,333, a 5-HT₄R agonist, was not effective as a prophylactic against fear and depressive-like behavior in female mice (Chen et al., 2020). However, RS-67,333 (10 mg/kg) was effective as a prophylactic against hyponeophagia. Therefore, it remained to be determined if combined (R,S)-ketamine and a 5-HT₄R agonists such as prucalopride could have additive effects.

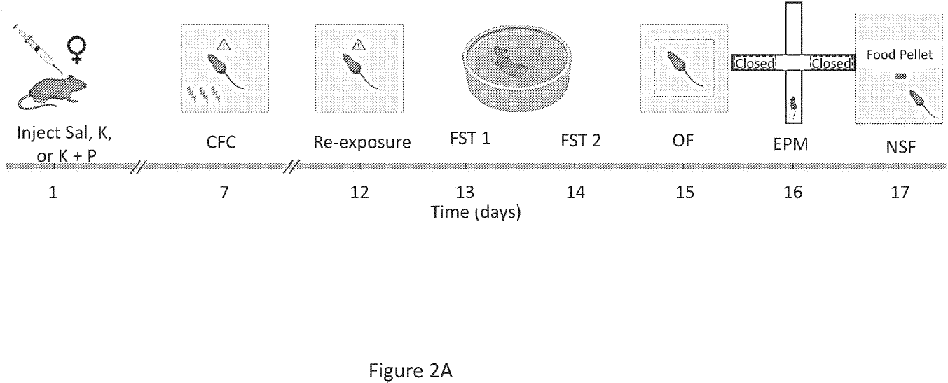

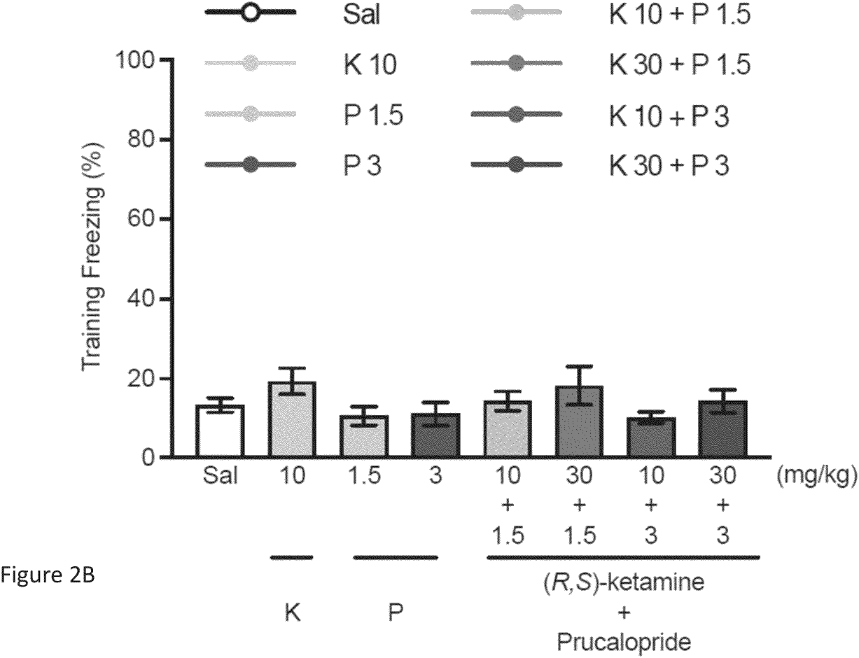

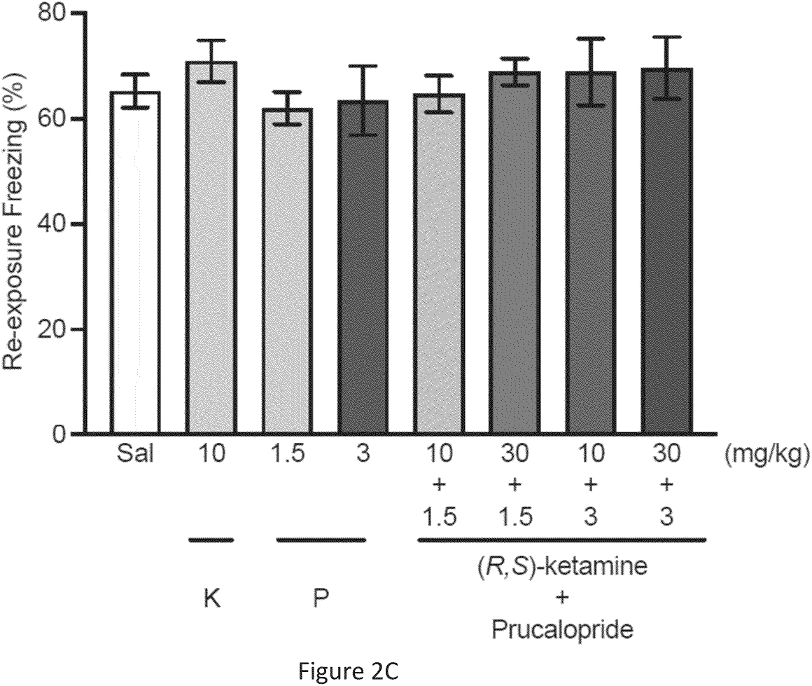

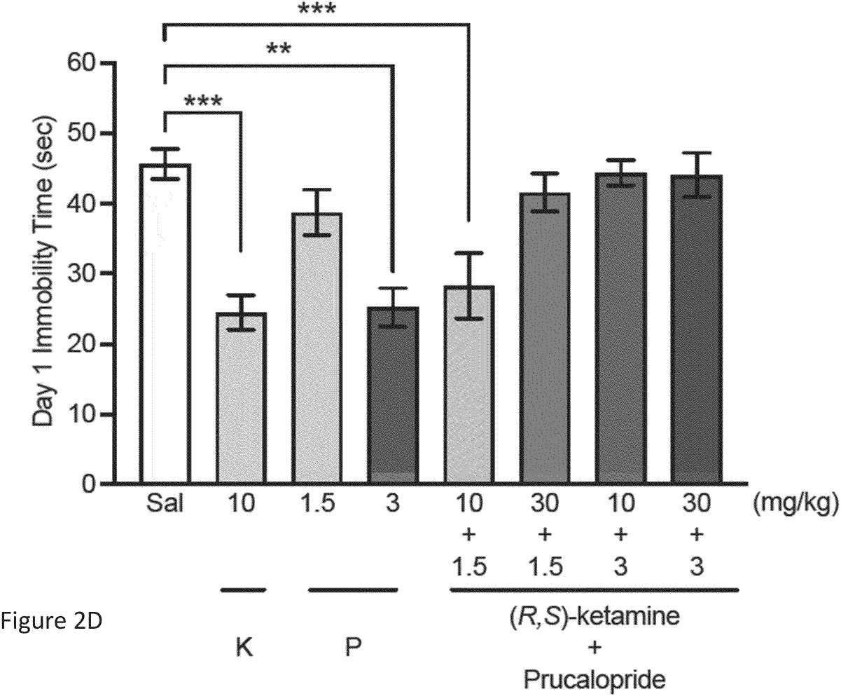

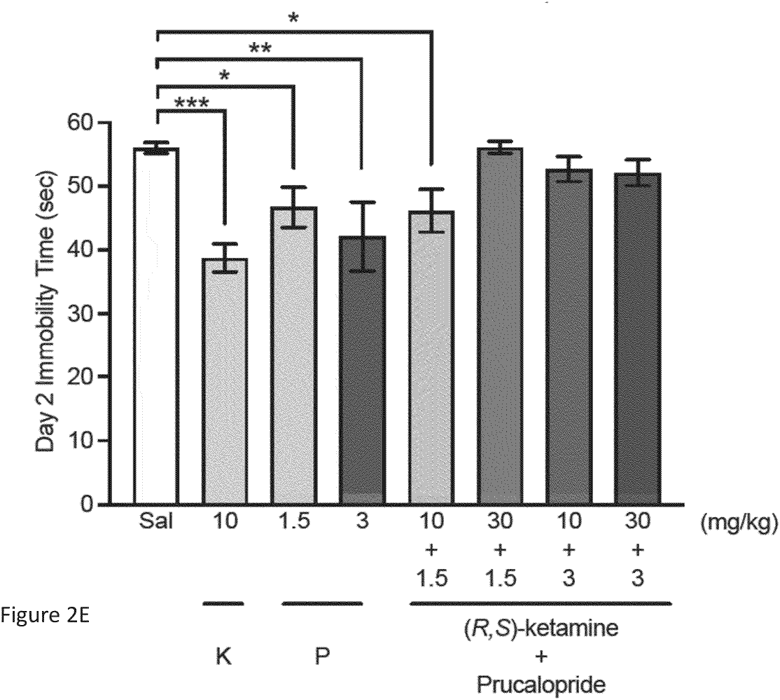

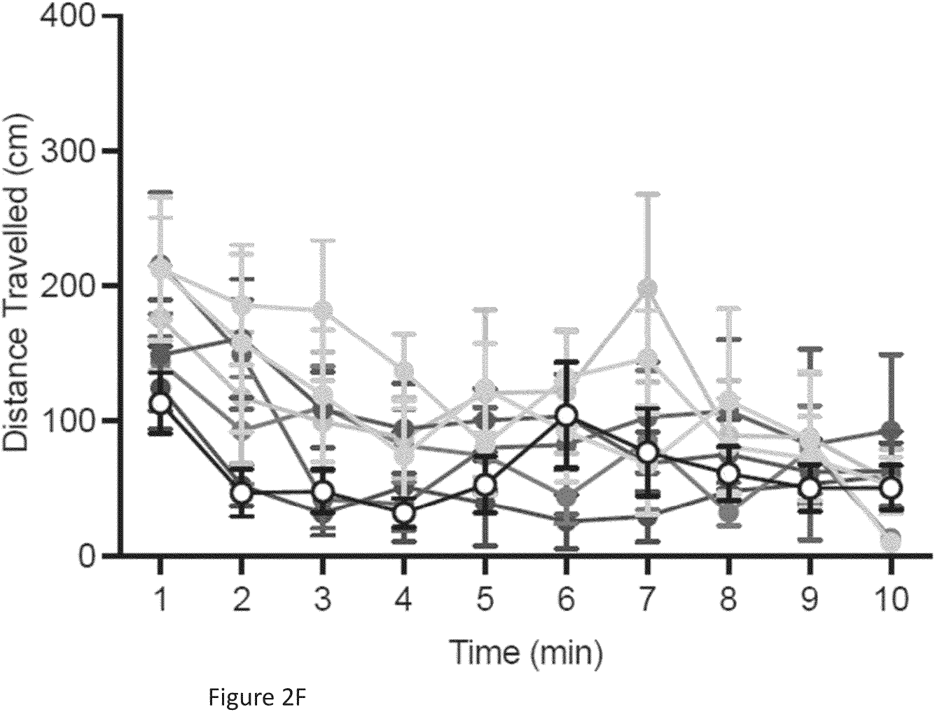

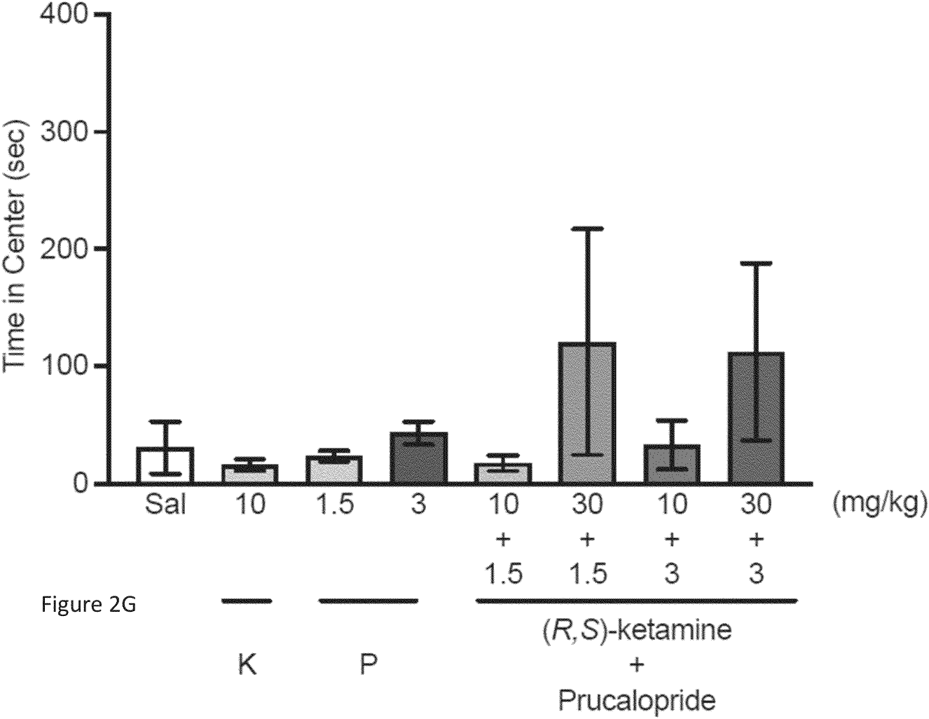

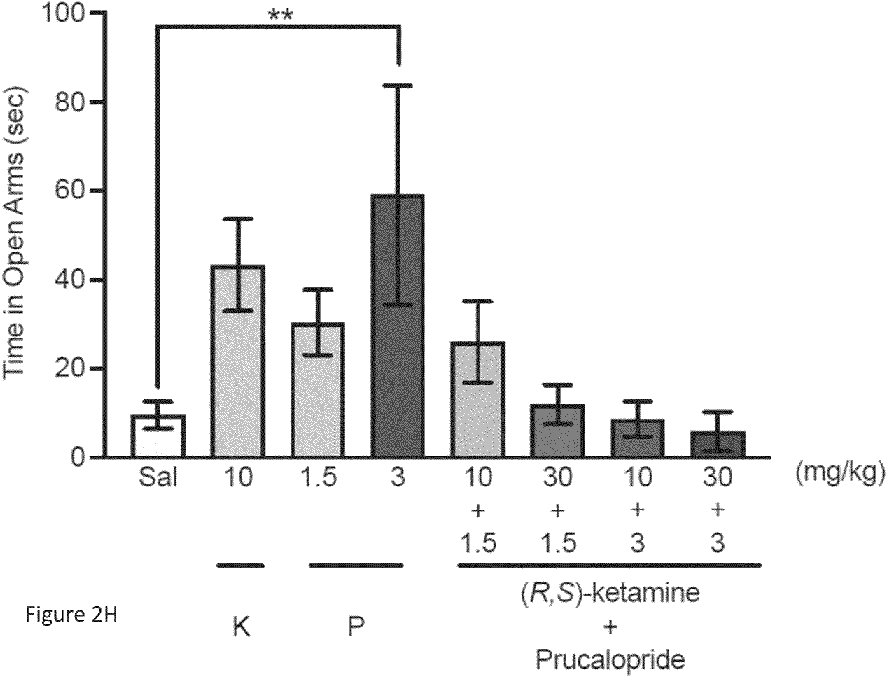

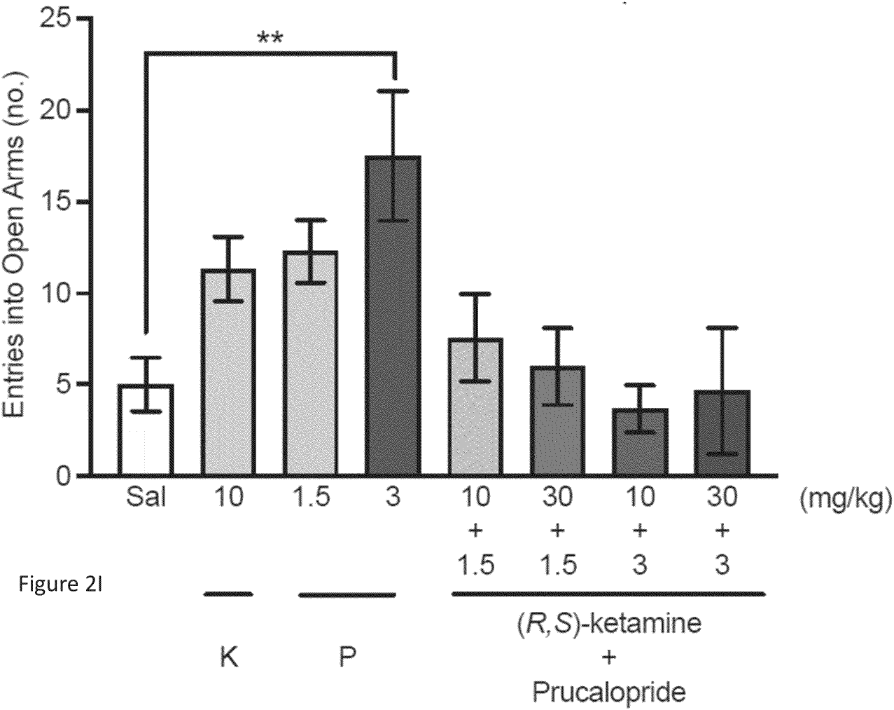

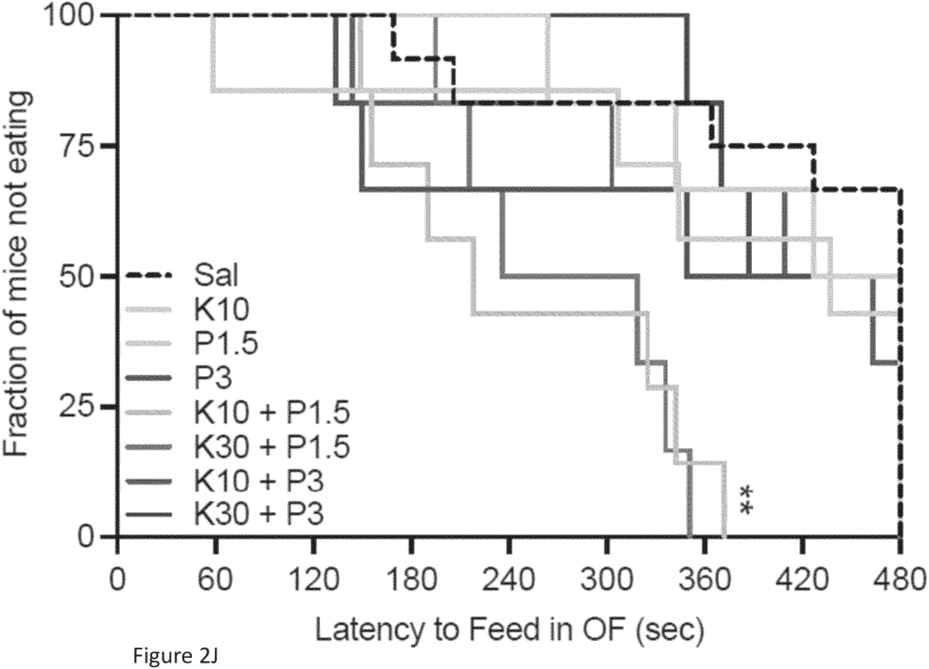

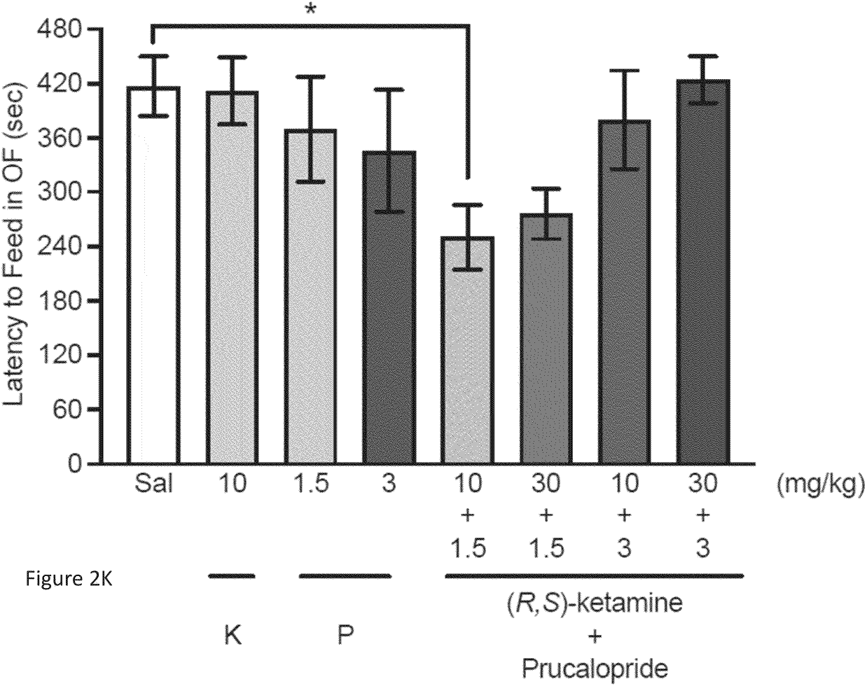

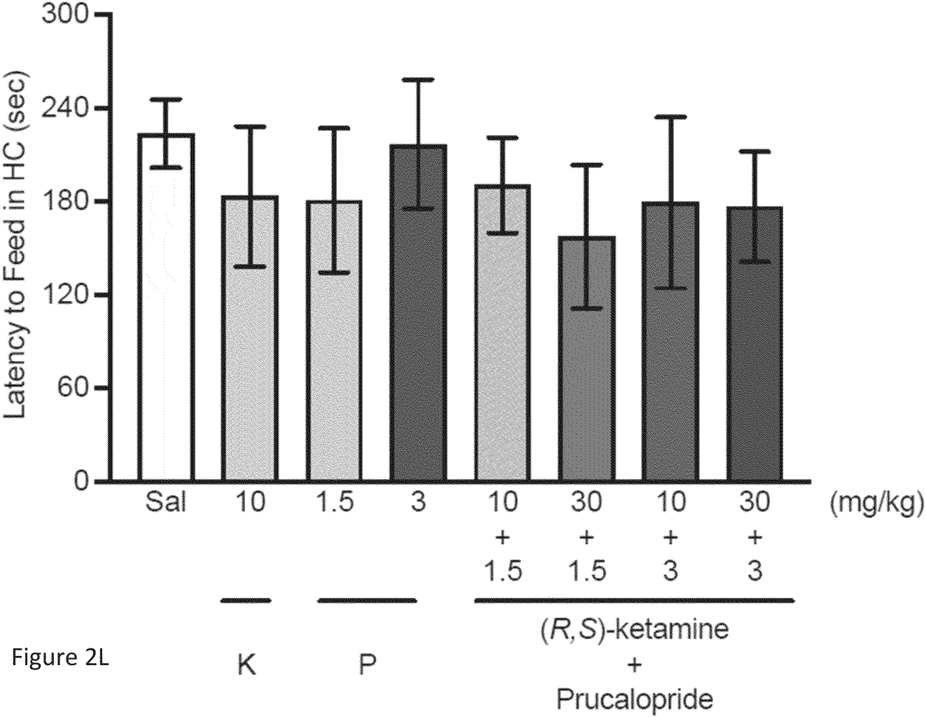

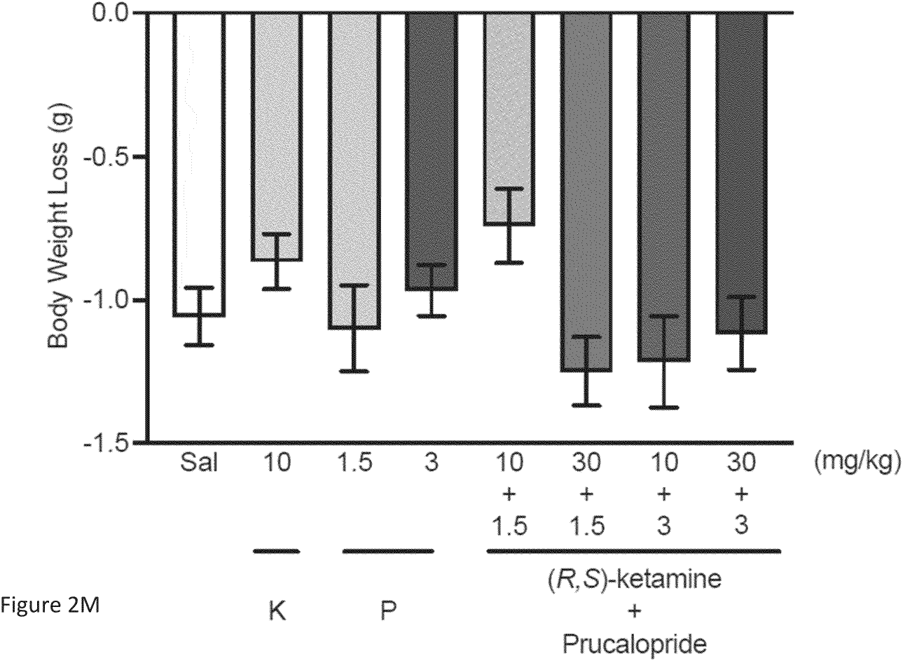

Female mice were injected with saline, (R,S)-ketamine, prucalopride, or combined (R,S)-ketamine and prucalopride. Five days later mice were administered a 3-shock CFC paradigm followed by behavior testing for fear, depressive-, and anxiety-like behavior using the forced swim test (FST), open field (OF), elevated plus maze (EPM), and novelty-suppressed feeding (NSF). See FIG. 2A.

All groups had comparable freezing during CFC training (FIG. 2B) and during re-exposure to the CFC context (FIG. 2C).

During days 1 and 2 of the FST, (R,S)-ketamine (10 mg/kg), prucalopride (1.5 and 3 mg/kg), or combined (R,S)-ketamine and prucalopride (10 + 1.5 mg/kg) reduced immobility time (FIGS. 2D and 2E).

All groups traveled a comparable distance during the OF (FIG. 2F) and spent a comparable amount of time in the center of the OF (FIG. 2G). However, prucalopride (3 mg/kg) increased the time in the open arms (FIG. 2H) and the number of entries into the open arms of the EPM (FIG. 2I).

In the NSF paradigm, prior work indicated that RS-67,333 was effective at reducing hyponeophagia in female 129S6/SvEv mice. However, here all groups had a similar latency to eat in the OF except mice administered combined (R,S)-ketamine and prucalopride (10 + 1.5 mg/kg) (FIGS. 2J and 2K). Combined (R,S)-ketamine and prucalopride (10 + 1.5 mg/kg) administration reduced the latency to feed in the OF. The latency to eat in the home cage (FIG. 2L) and the weight loss (%) (FIG. 2M) did not differ between the groups.

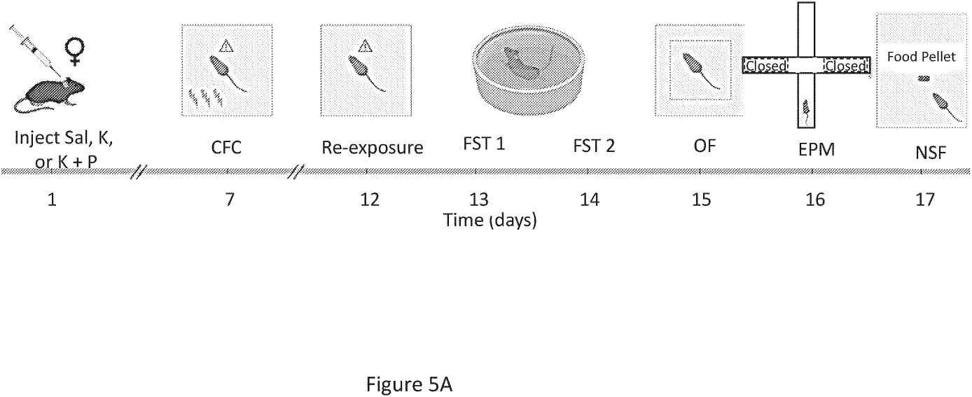

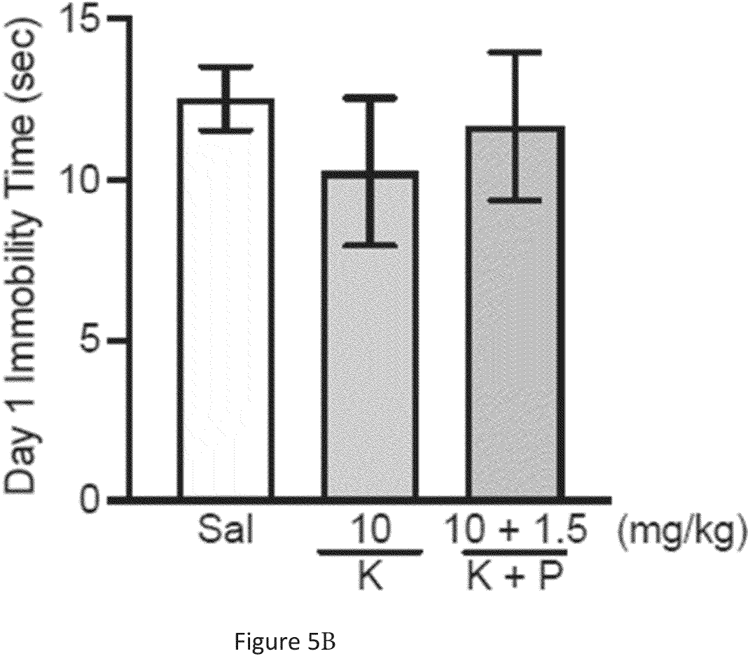

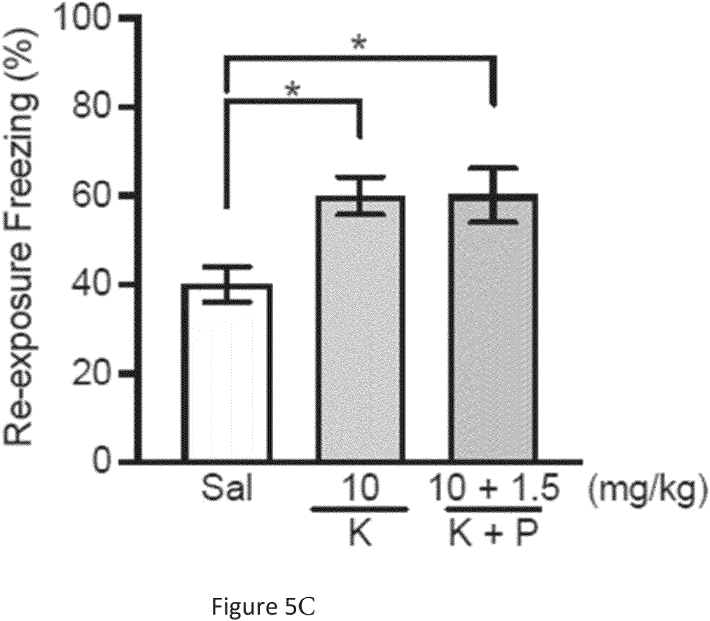

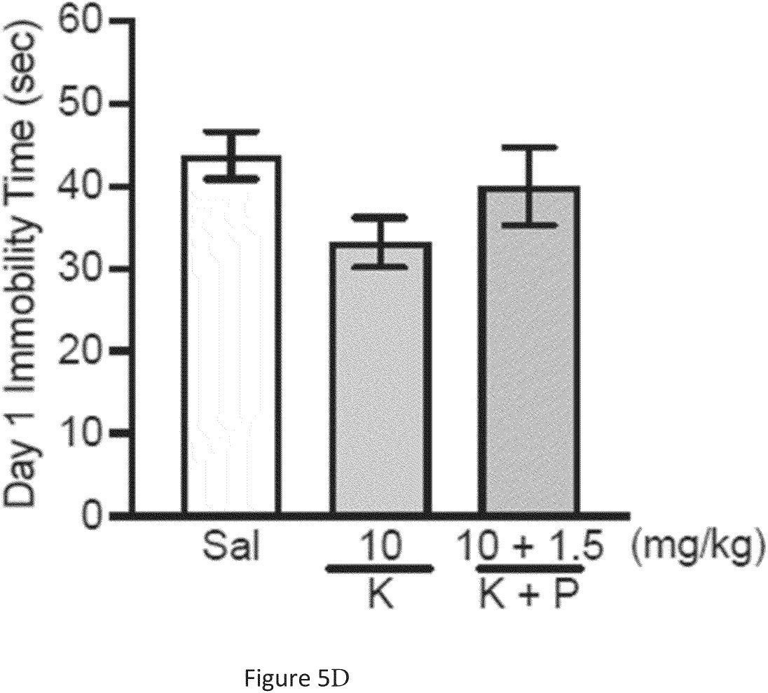

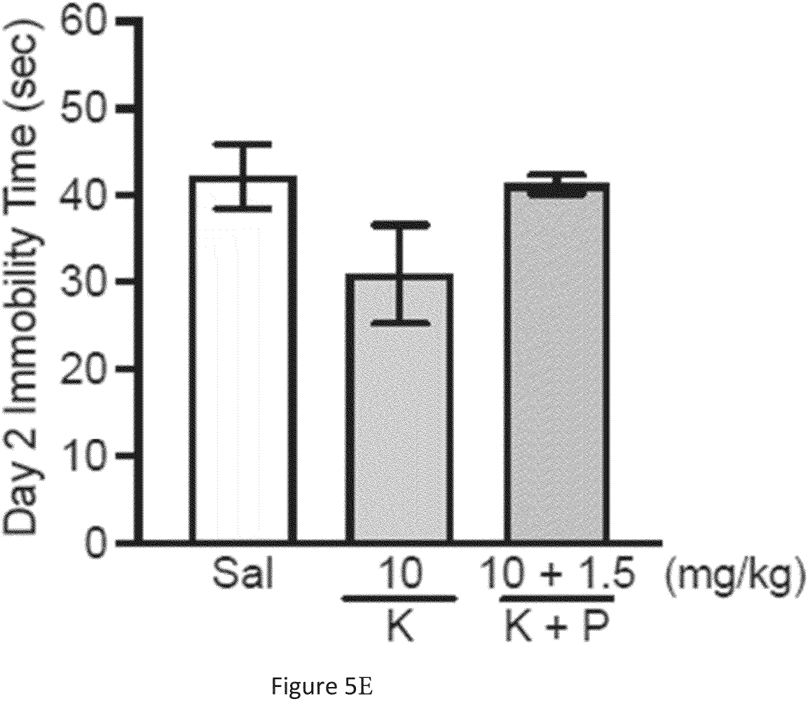

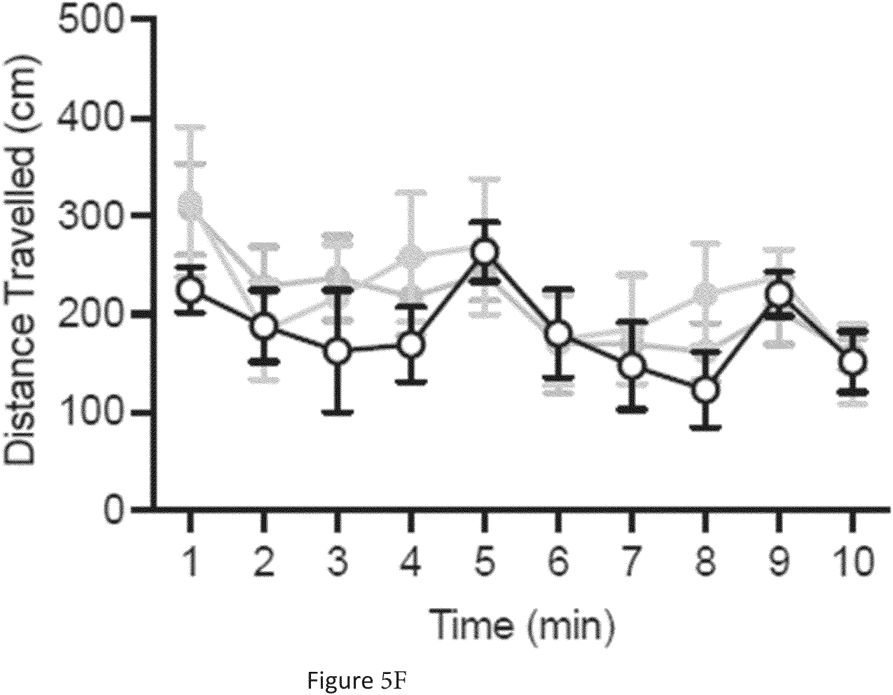

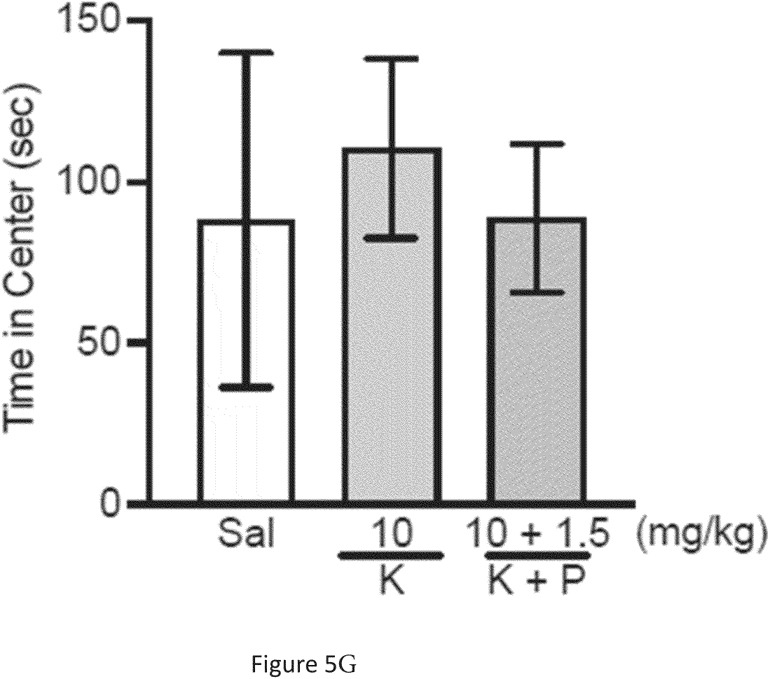

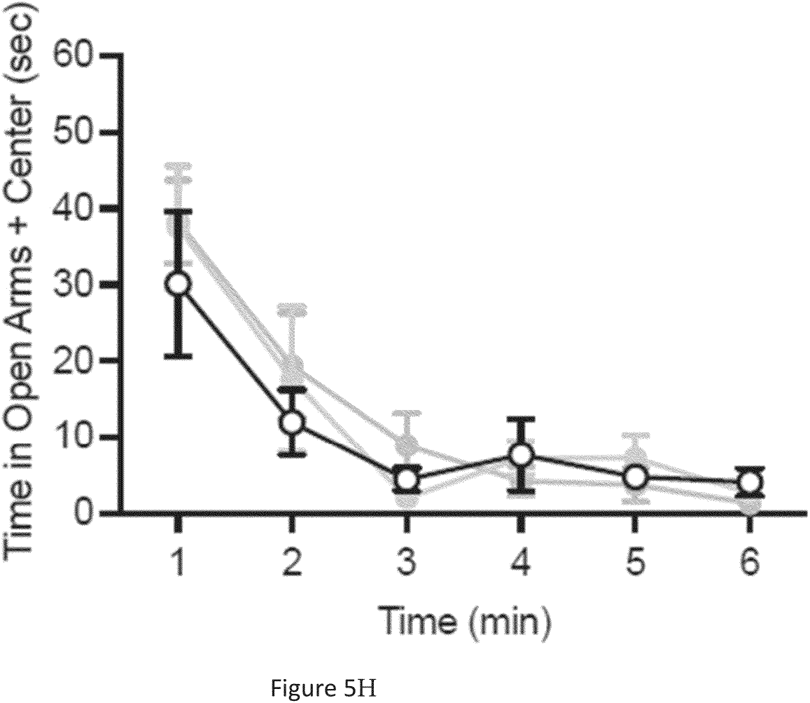

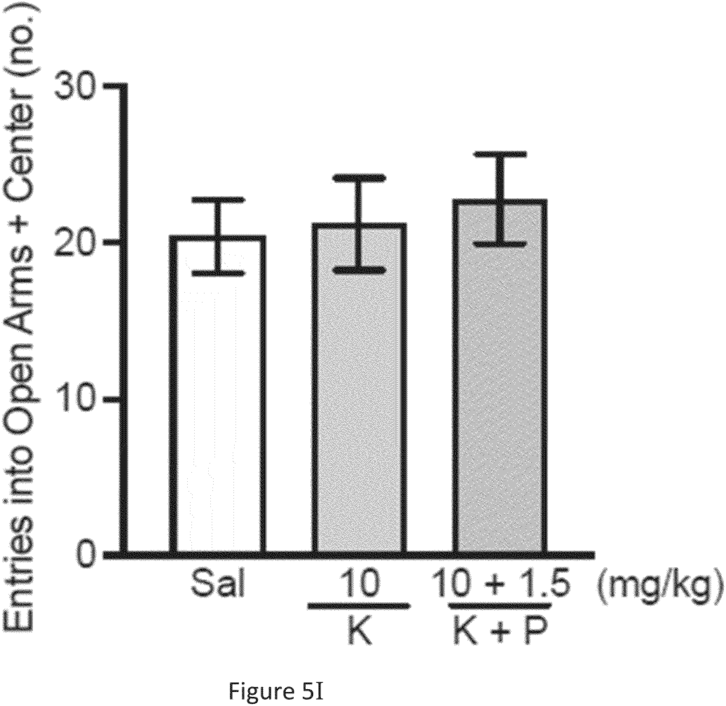

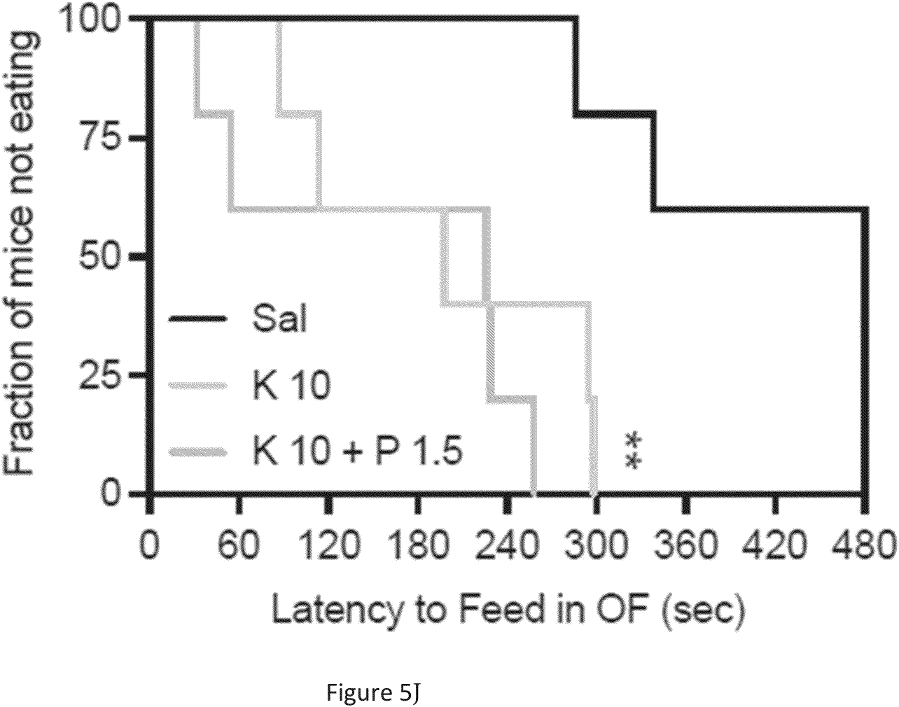

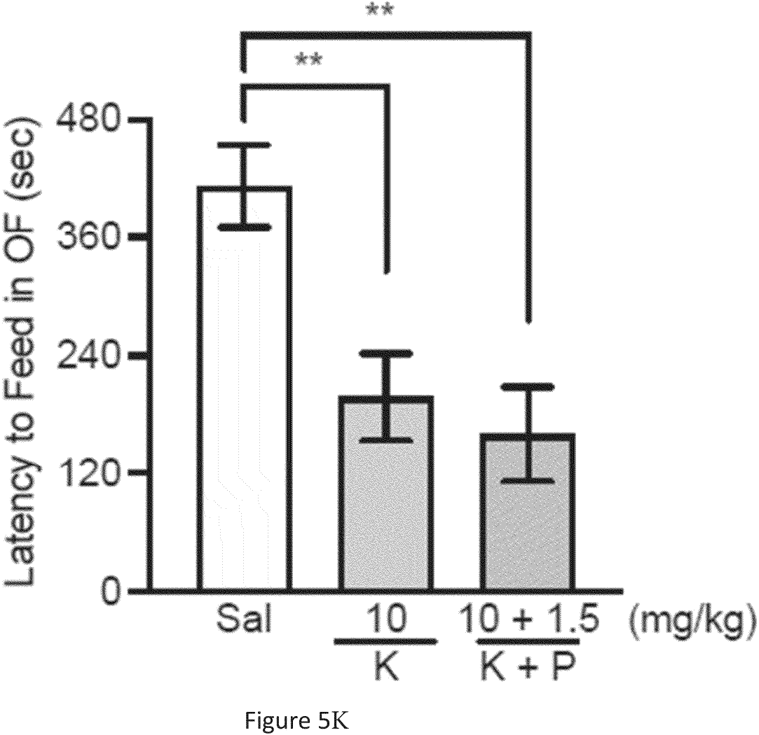

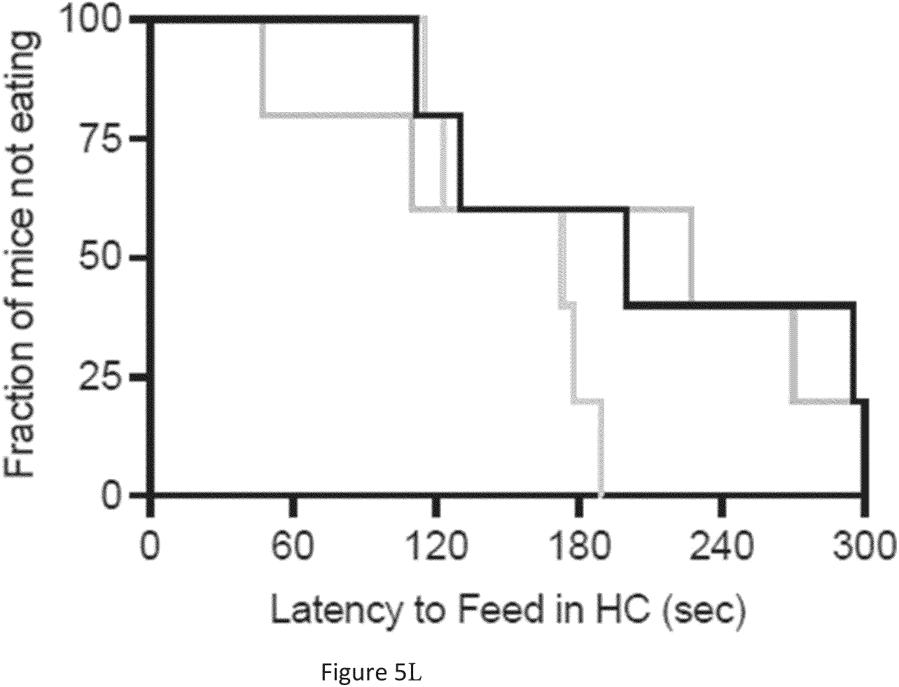

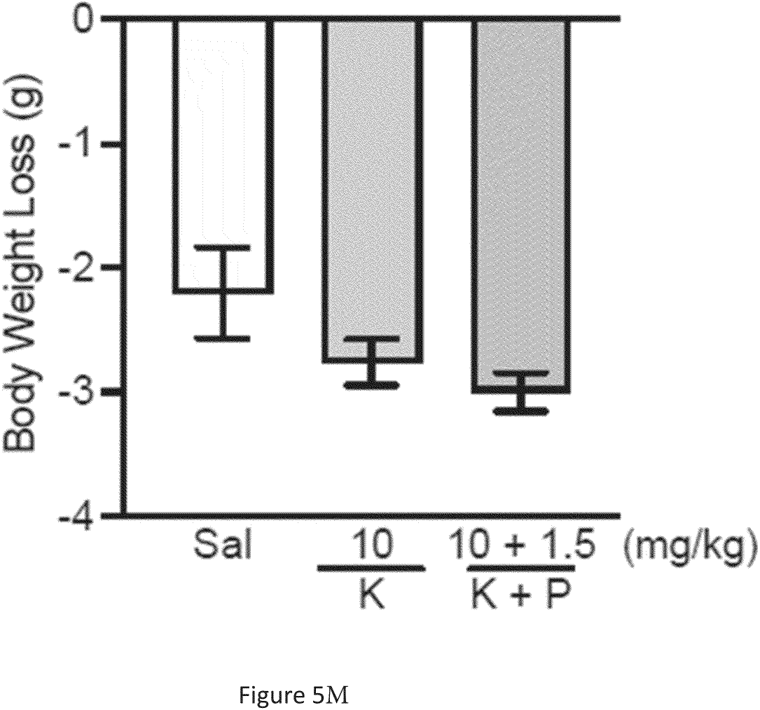

Similar results were seen in another female mouse model (FIG. 5). Combined (R,S)-ketamine and prucalopride (10 + 1.5 mg/kg) administration reduced the latency to feed in the OF (FIGS. 5J and 5K). The latency to eat in the home cage (FIG. 5L) and food not eaten (FIG. 5M) did not differ between the groups.

### Example 4 - Combined Prophylactic (R,S)-Ketamine and Prucalopride Decreased Fear and, Depressive-Like Behavior, i.e., Protects Against Stress in Male Mice When Administered After Stress

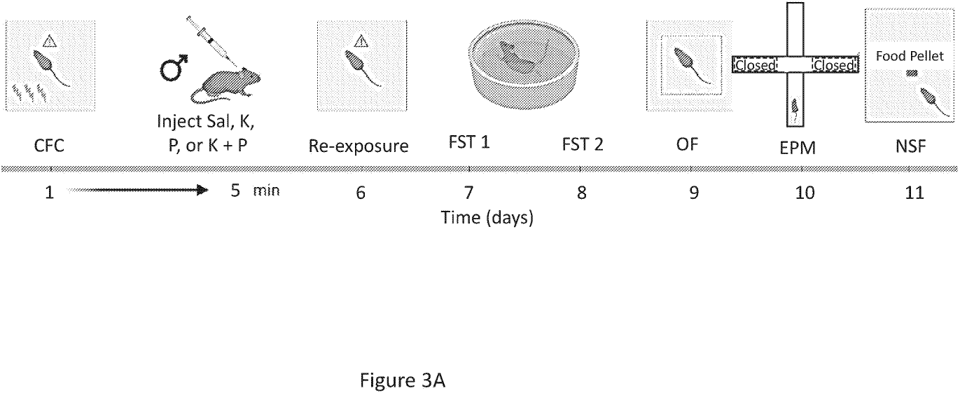

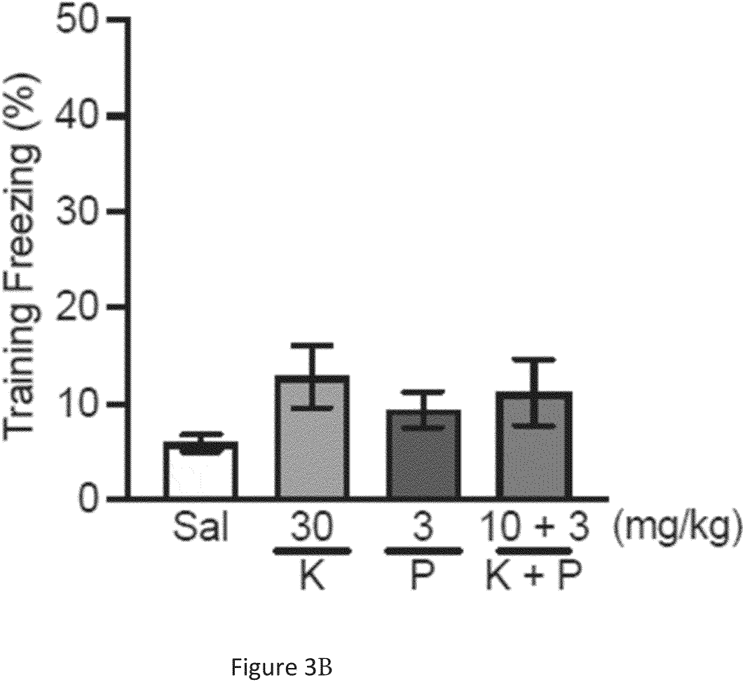

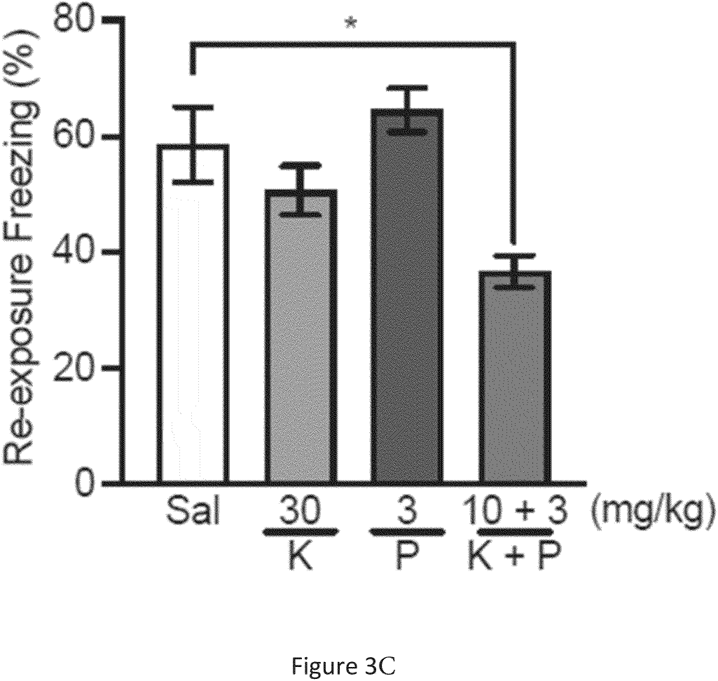

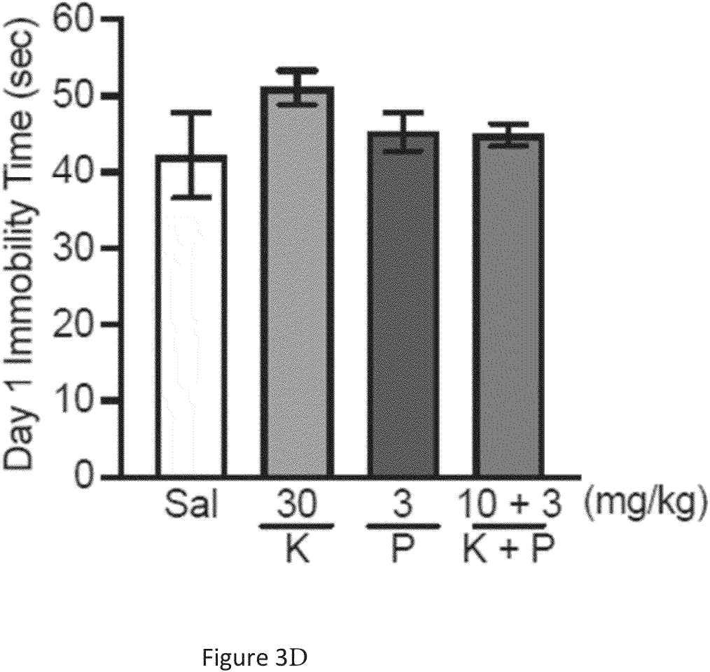

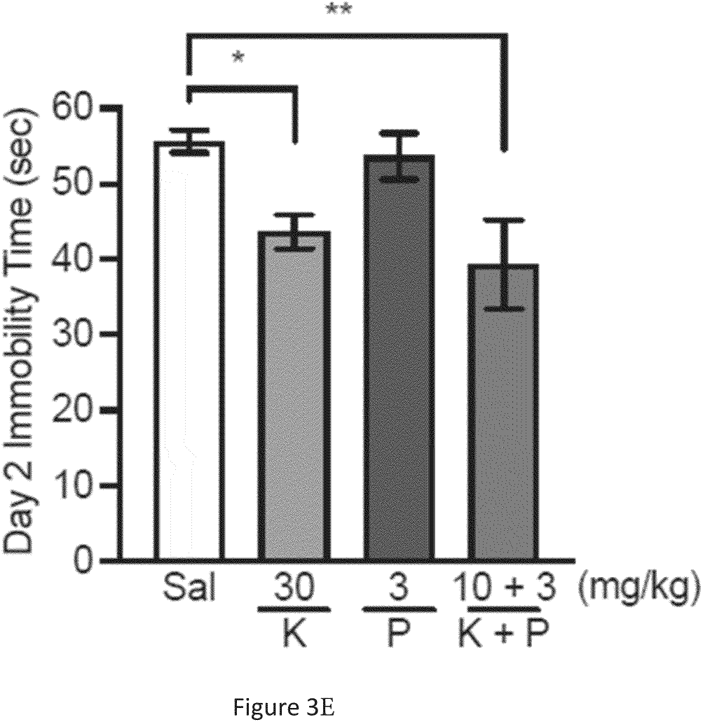

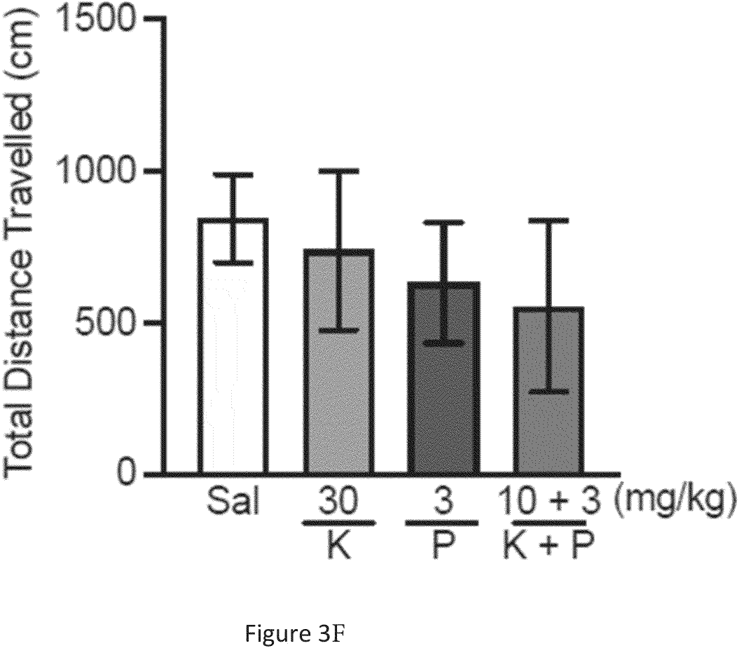

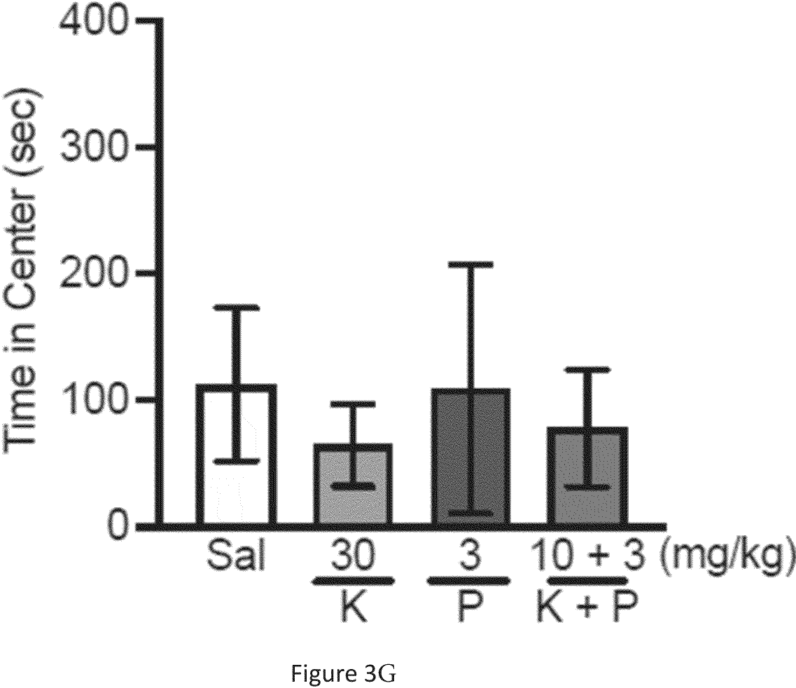

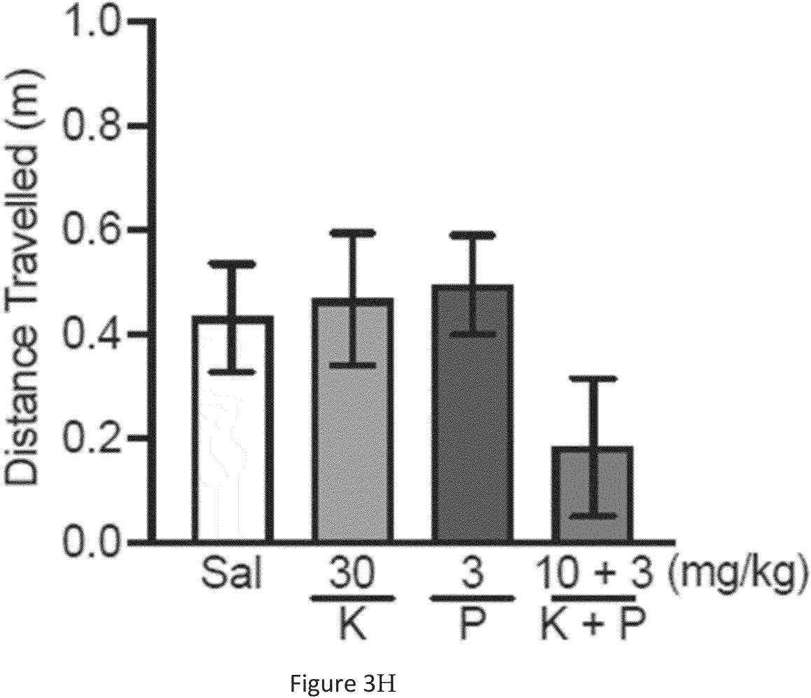

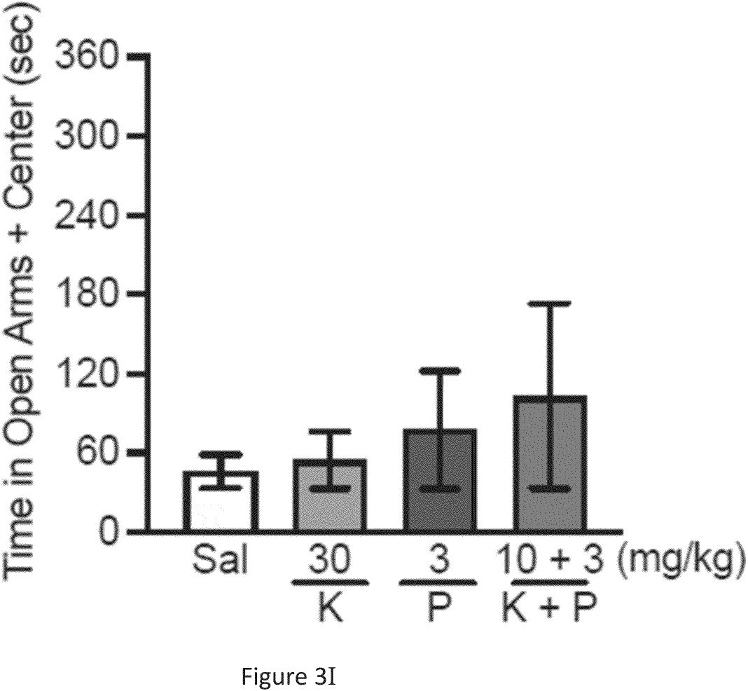

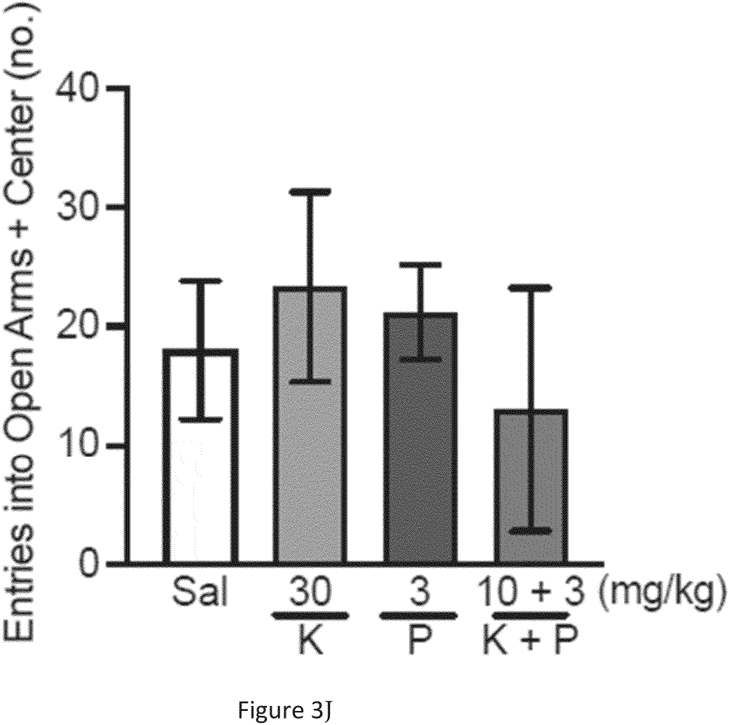

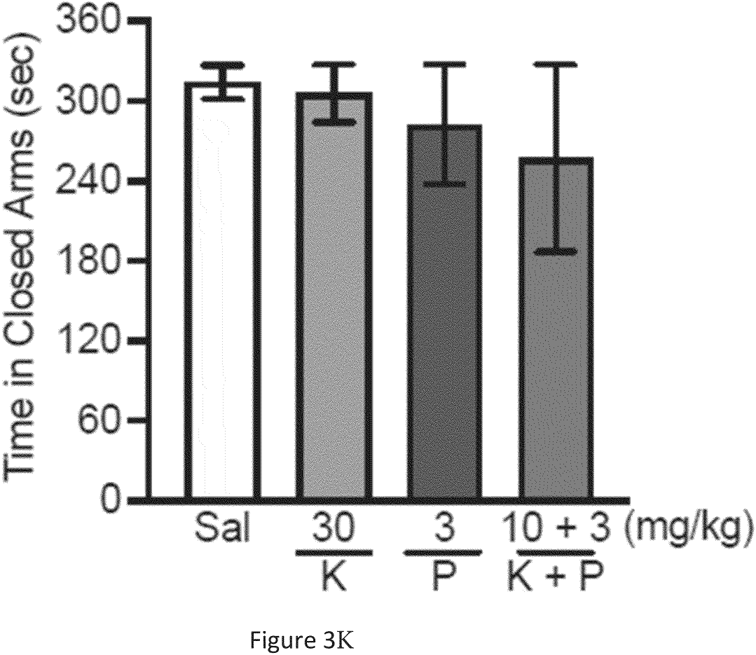

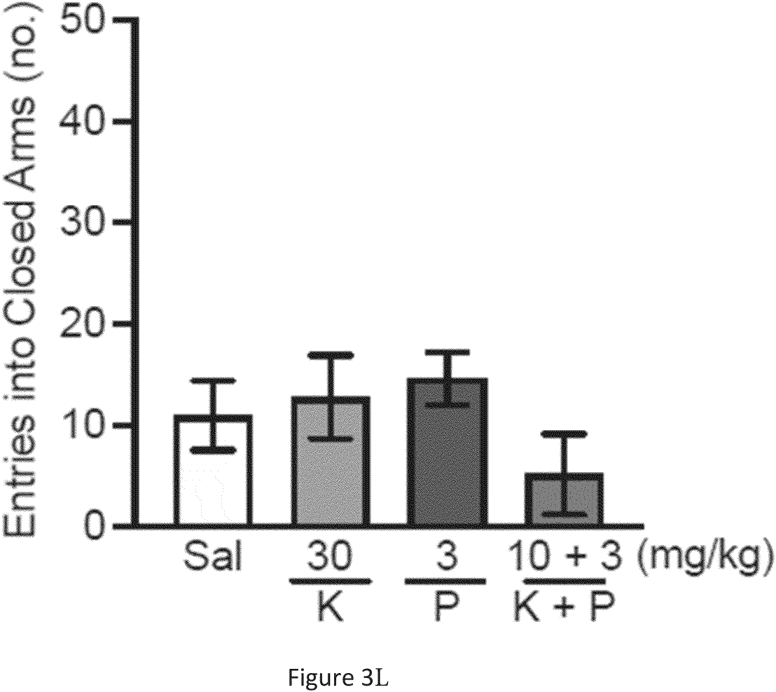

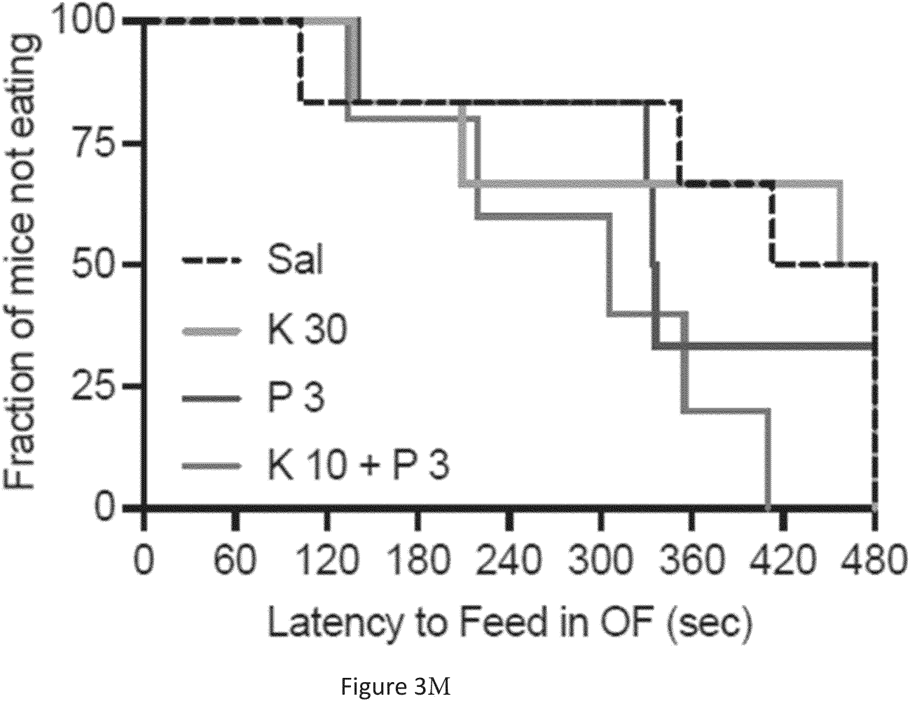

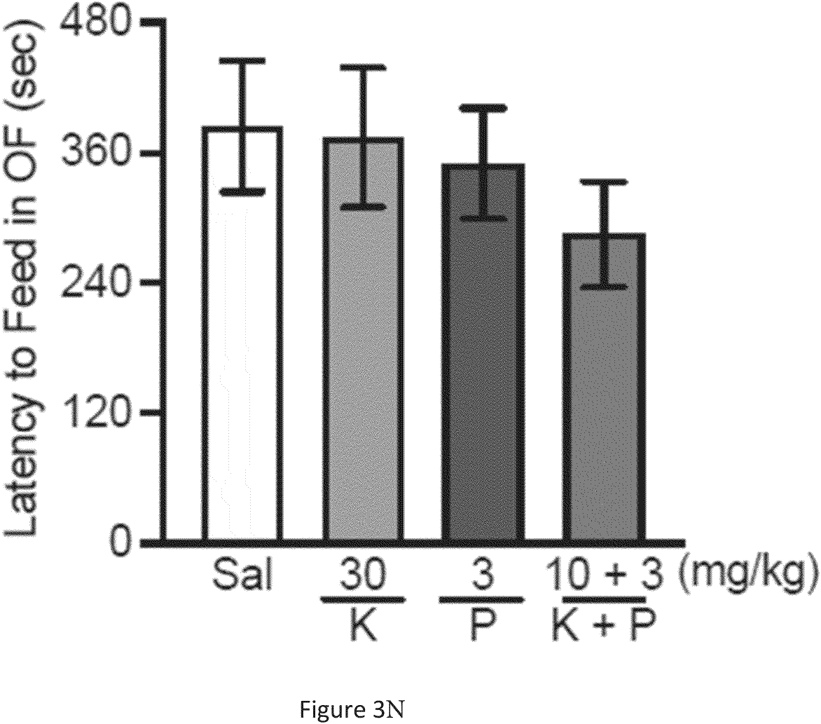

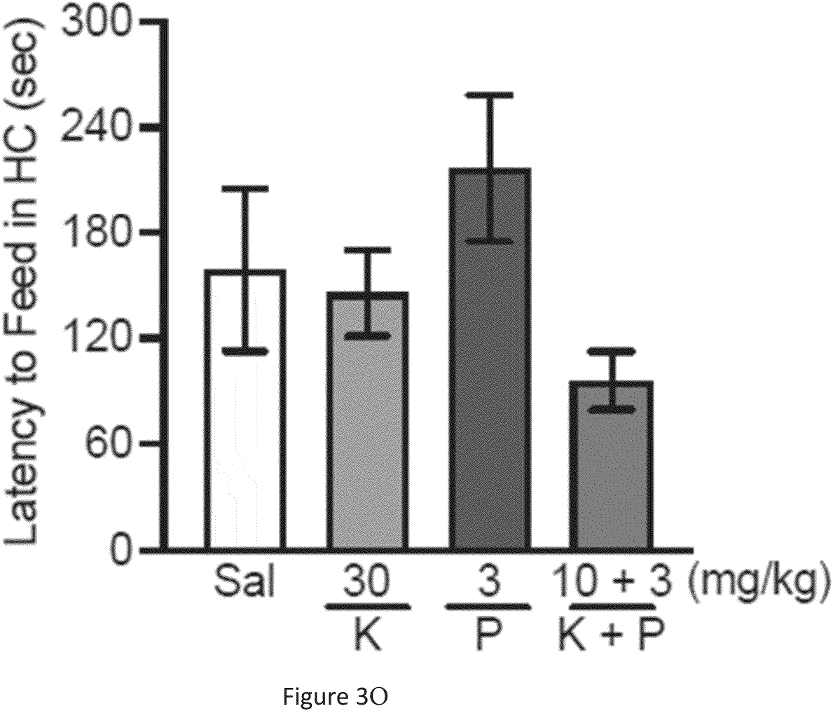

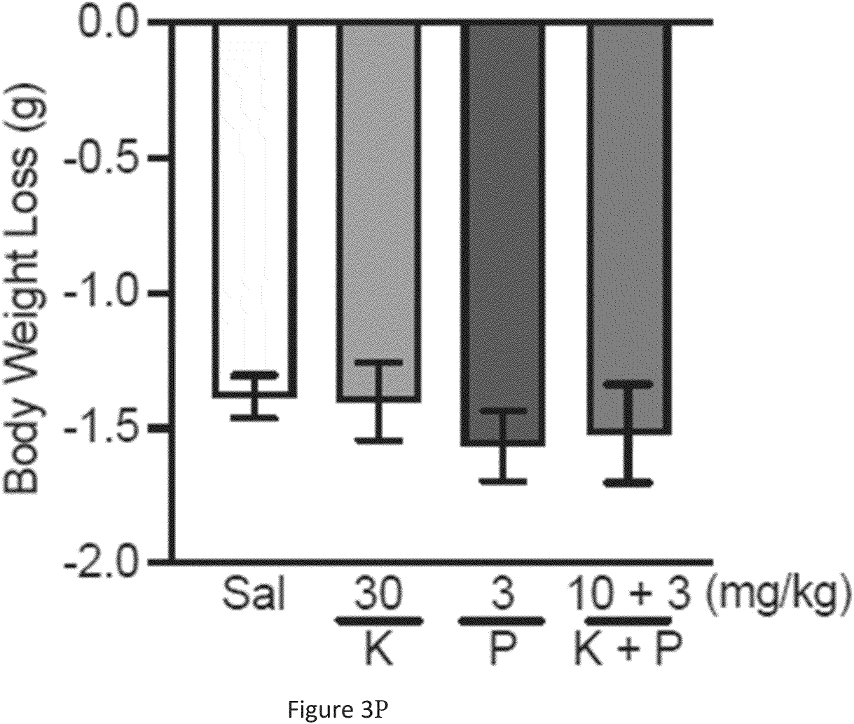

Male mice were injected with saline, (R,S)-ketamine, prucalopride, or combined (R,S)-ketamine and prucalopride five minutes after a 3-shock CFC paradigm followed by behavior testing for fear, depressive-, and anxiety-like behavior using the forced swim test (FST), open field (OF), elevated plus maze (EPM), and novelty-suppressed feeding (NSF). See FIG. 3A.

All groups had comparable freezing during CFC training (FIG. 3B). However, during re-exposure to the CFC context (R,S)-ketamine and prucalopride (10 + 3 mg/kg) significantly reduced freezing, i.e., effective at decreasing fear, when compared with saline (FIG. 3C).

During day 1 of the FST, all groups had comparable immobility time (FIG. 3D). Conversely, during day 2 of the FST, (R,S)-ketamine (30 mg/kg) and (R,S)-ketamine and prucalopride (10 + 3 mg/kg) significantly reduced behavioral despair when compared with saline (FIG. 3E).

Behavior in all groups was comparable in the OF, EPM and NSF assays. See FIGS. 3F- 3P.

These data indicated that the combined dosing of (R,S)-ketamine and prucalopride attenuated learned fear and reduced behavioral despair even when administered after stress.

### Example 5 - Combined Prophylactic (R,S)-Ketamine and Prucalopride Decreased Fear and Depressive-Like Behavior, i.e., Protects Against Stress, in Female Mice When Administered After Stress

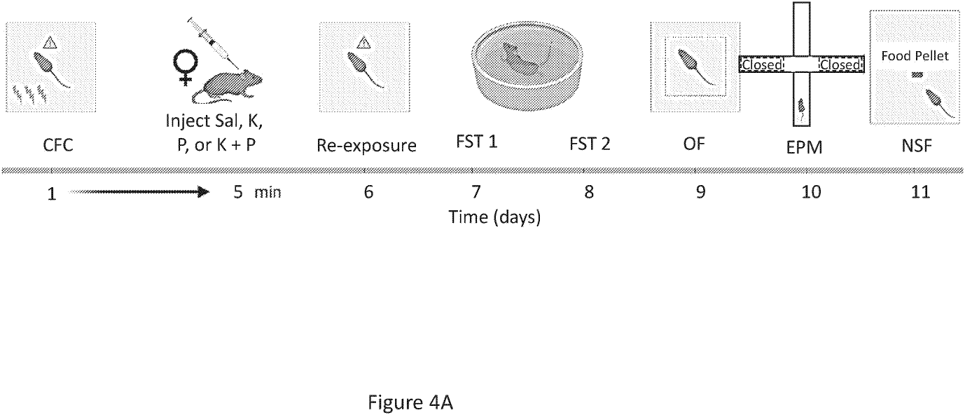

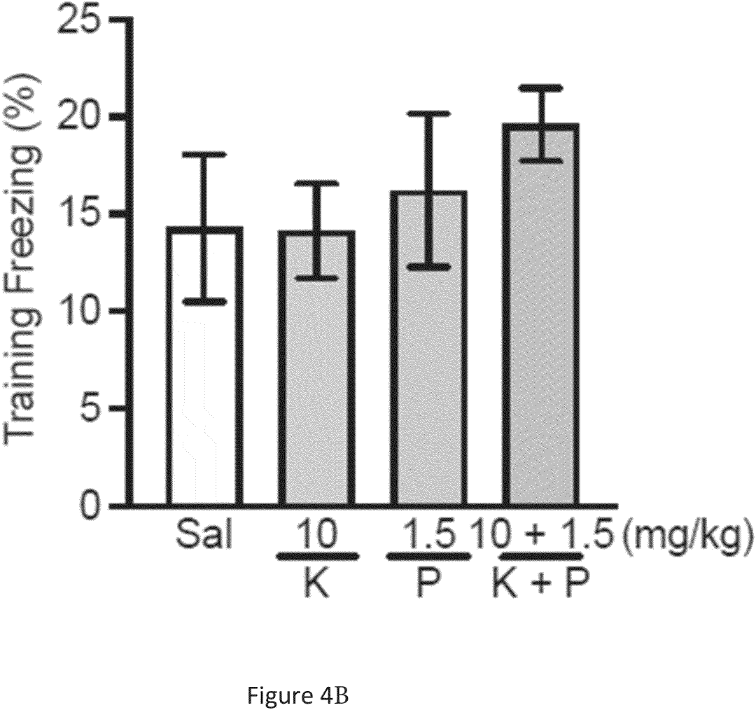

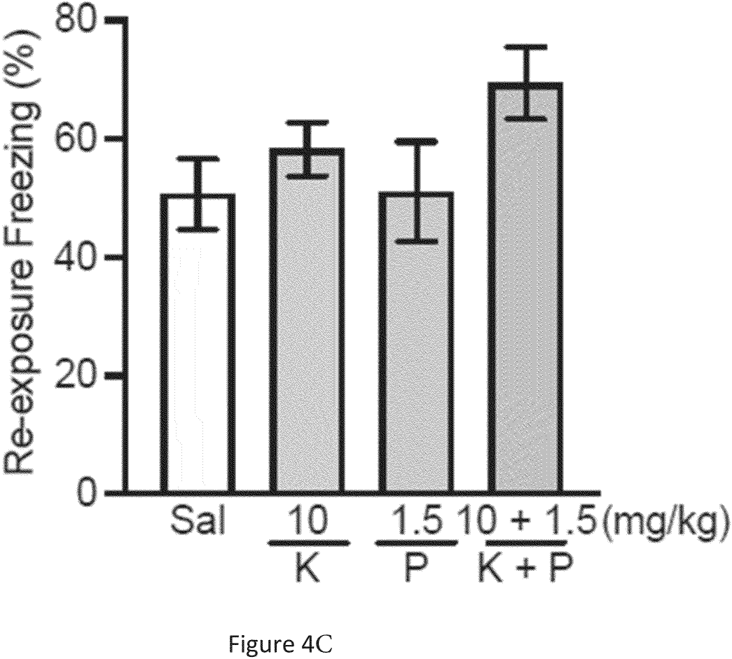

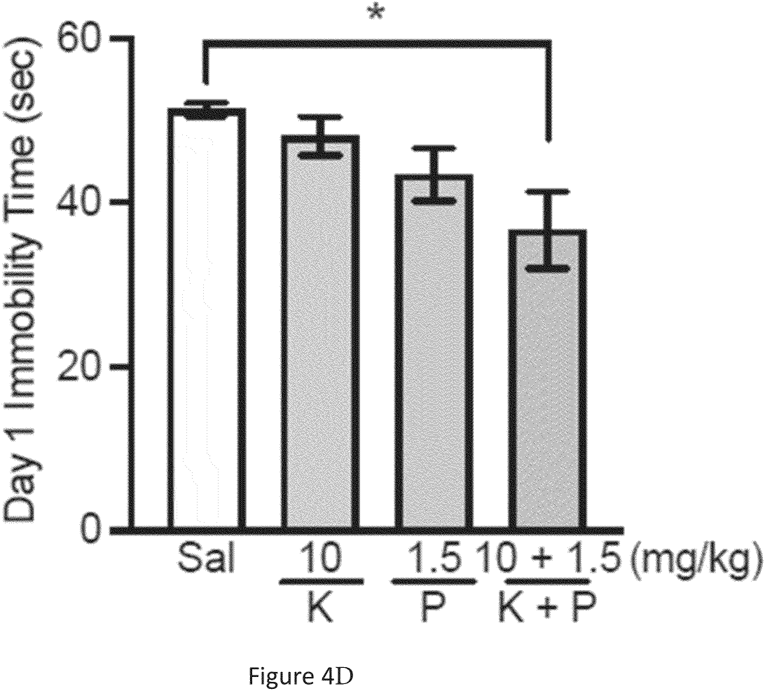

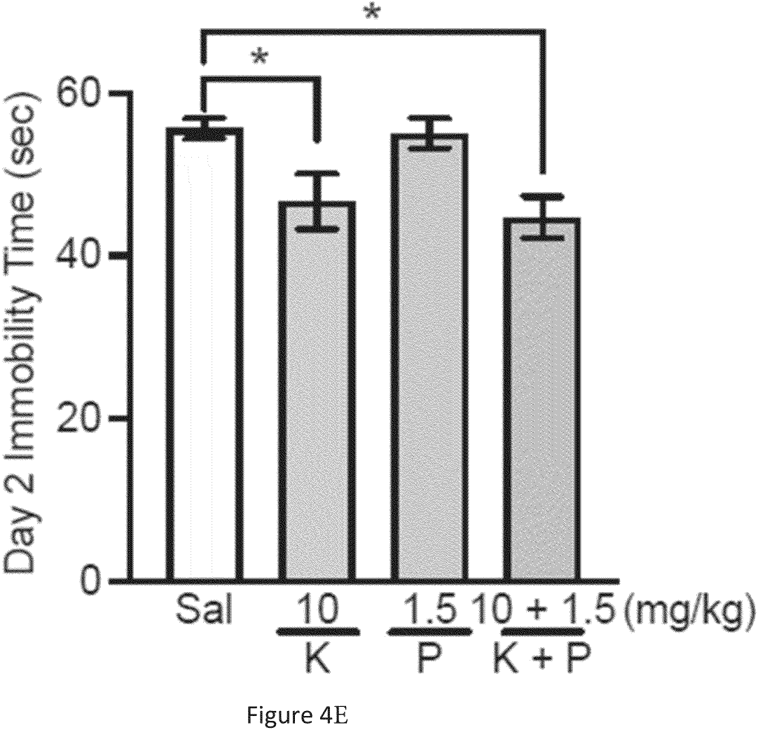

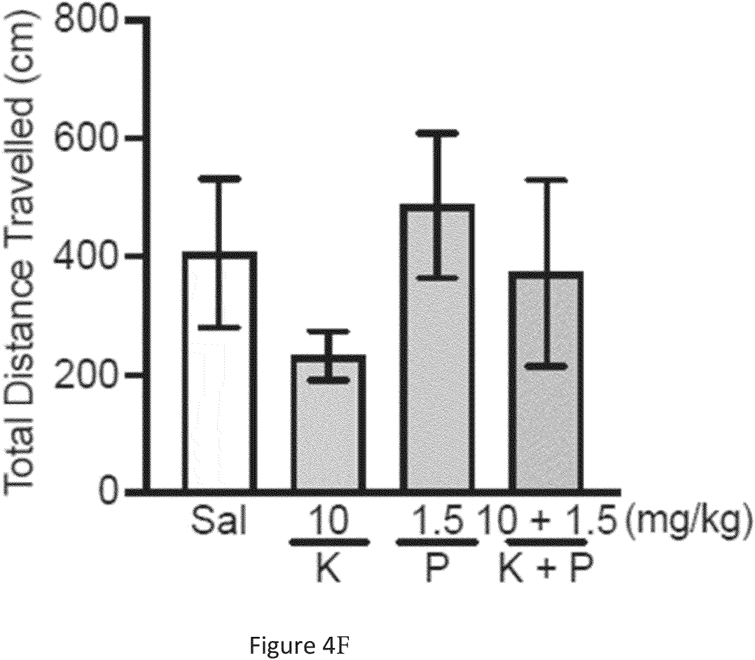

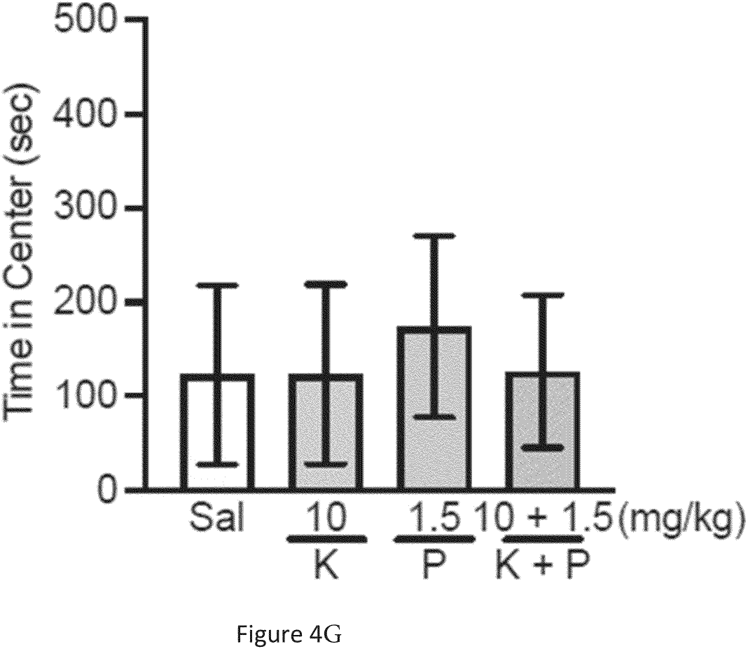

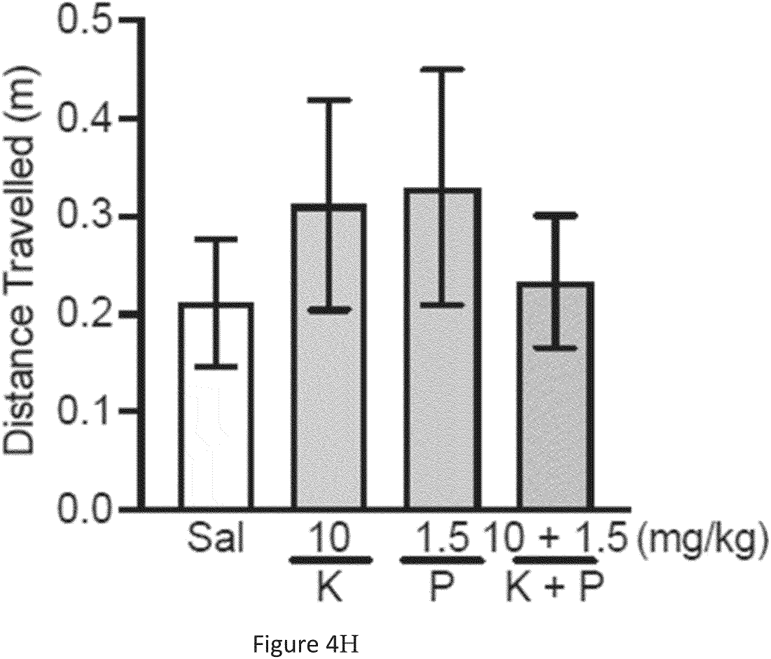

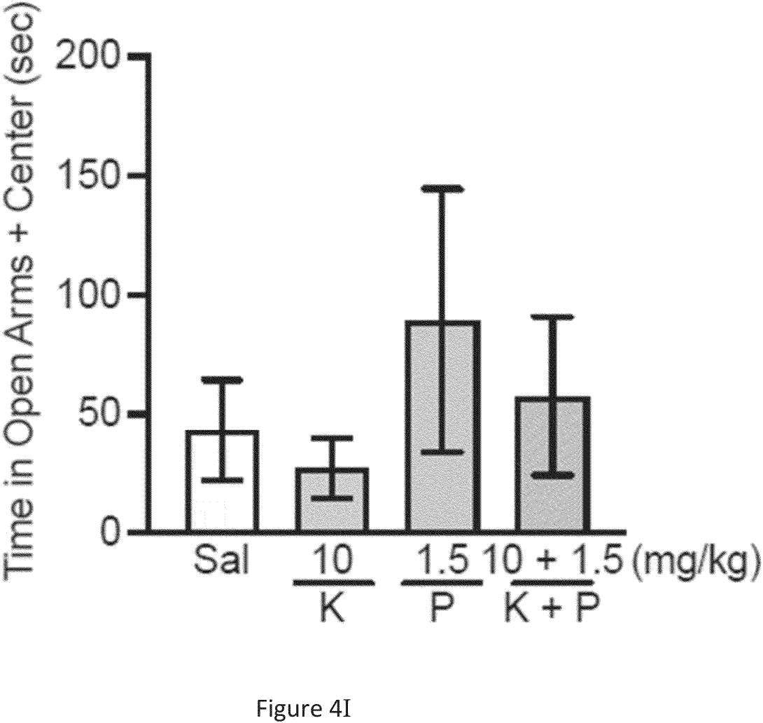

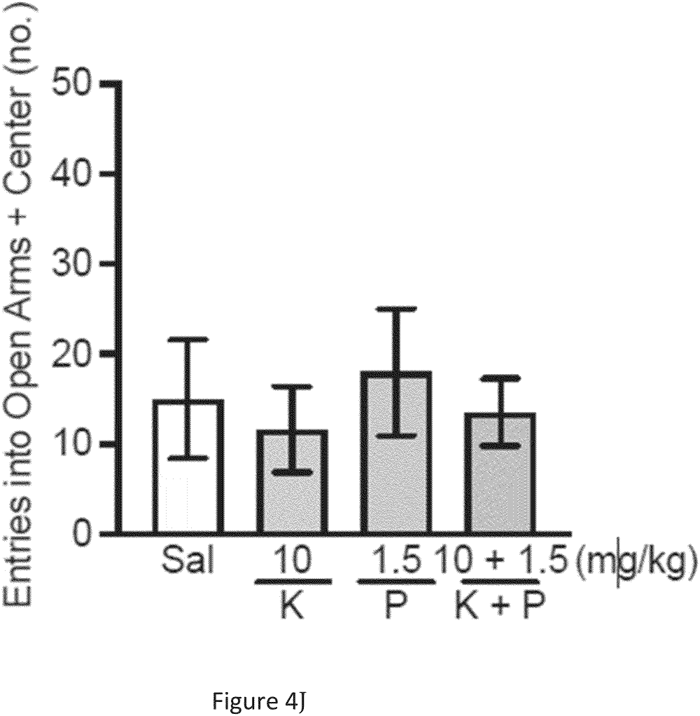

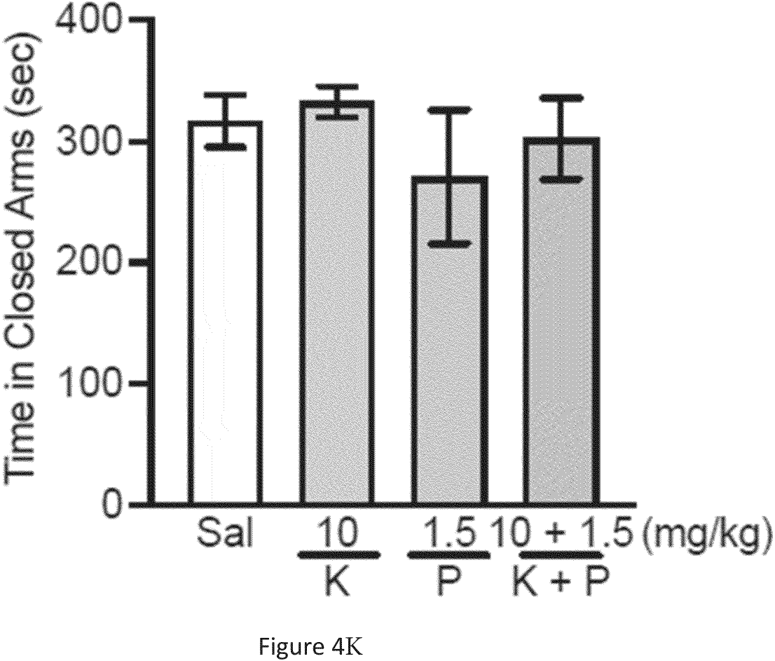

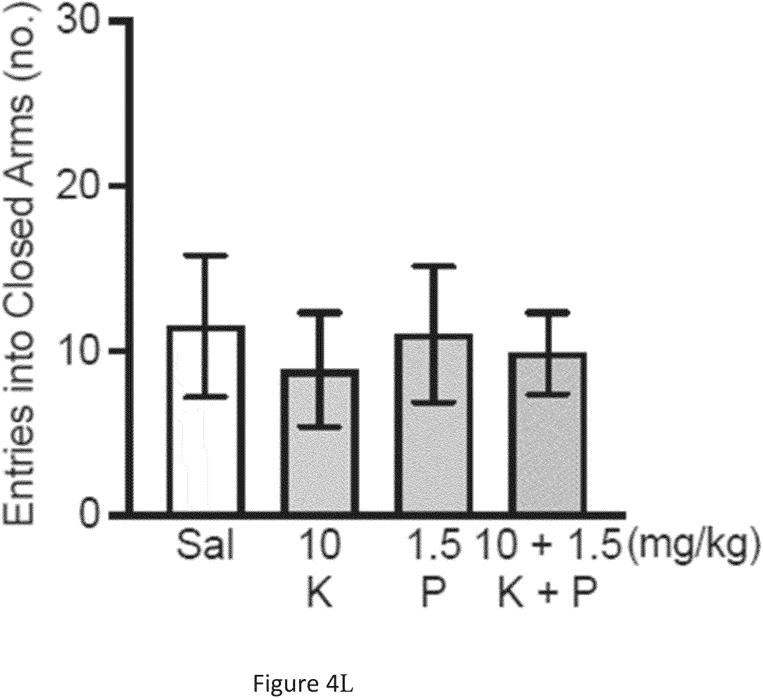

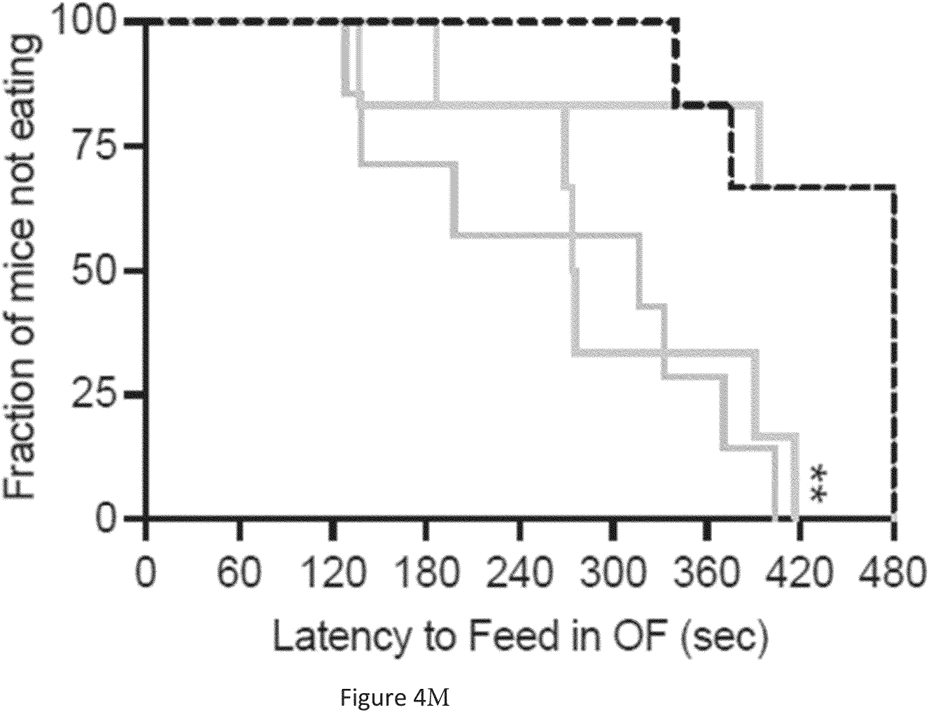

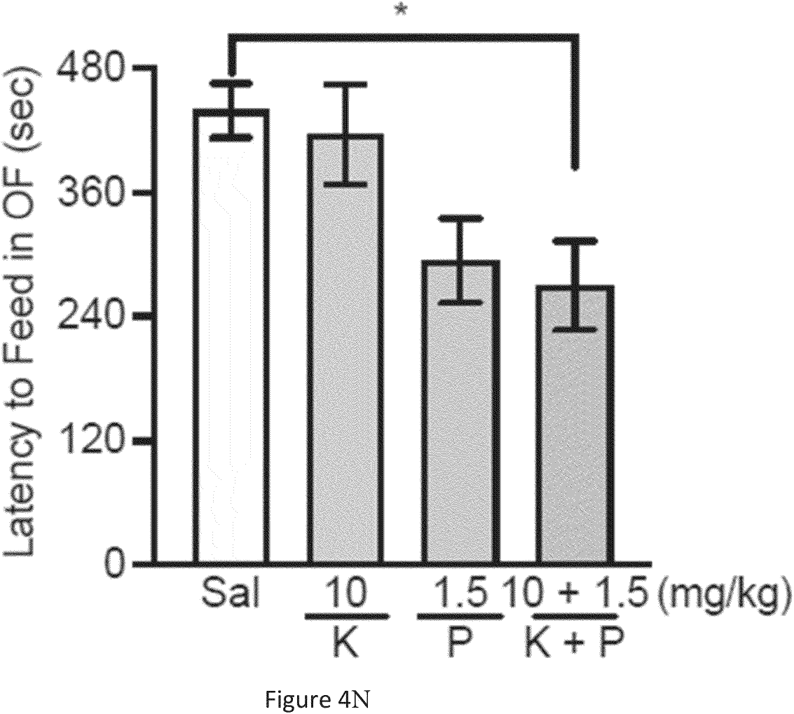

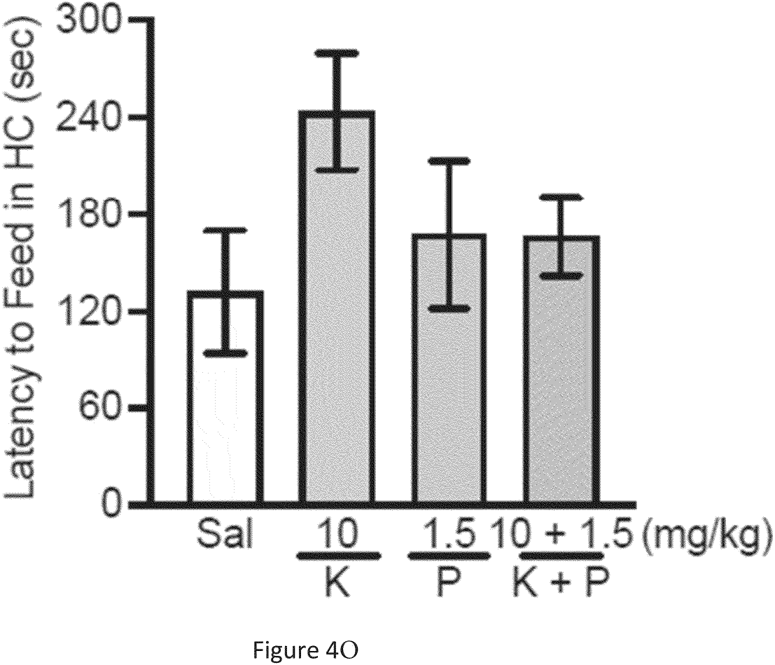

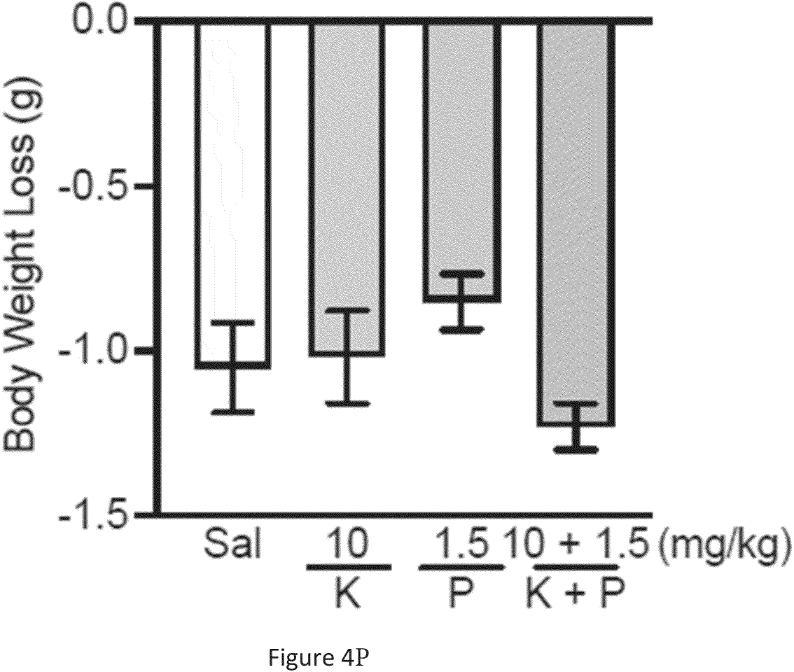

Female mice were injected with saline, (R,S)-ketamine, prucalopride, or combined (R,S)-ketamine and prucalopride five minutes before a 3-shock CFC paradigm followed by behavior testing for fear, depressive-, and anxiety-like behavior using the forced swim test (FST), open field (OF), elevated plus maze (EPM), and novelty-suppressed feeding (NSF). See FIG. 4A.

All groups had comparable freezing during CFC training (FIG. 4B) and during re-exposure to the CFC context (FIG. 4C).

During days 1 and 2 of the FST, combined (R,S)-ketamine and prucalopride (10 + 1.5 mg/kg) reduced immobility time (FIGS. 4D and 4E).

Behavior was comparable in all groups in the OF and EPM assays. See FIGS. 4F -4L

In the NSF assay, again all groups had a similar latency to eat in the OF except mice administered combined (R,S)-ketamine and prucalopride (10 + 1.5 mg/kg) (FIGS. 4M and 4N). Combined (R,S)-ketamine and prucalopride (10 + 1.5 mg/kg) administration reduced the latency to feed in the OF. The latency to eat in the home cage (FIG. 4O) and the weight loss (%) (FIG. 4P) did not differ between the groups.

These data indicated that the combined dosing of (R,S)-ketamine and prucalopride attenuated learned fear and reduced behavioral despair and decreased NSF behavior even when administered after stress.

### Example 6 - Combined (R,S)-Ketamine and Prucalopride Does not Alter Behavioral Despair and Perseverative Behavior in Non-Stressed Males

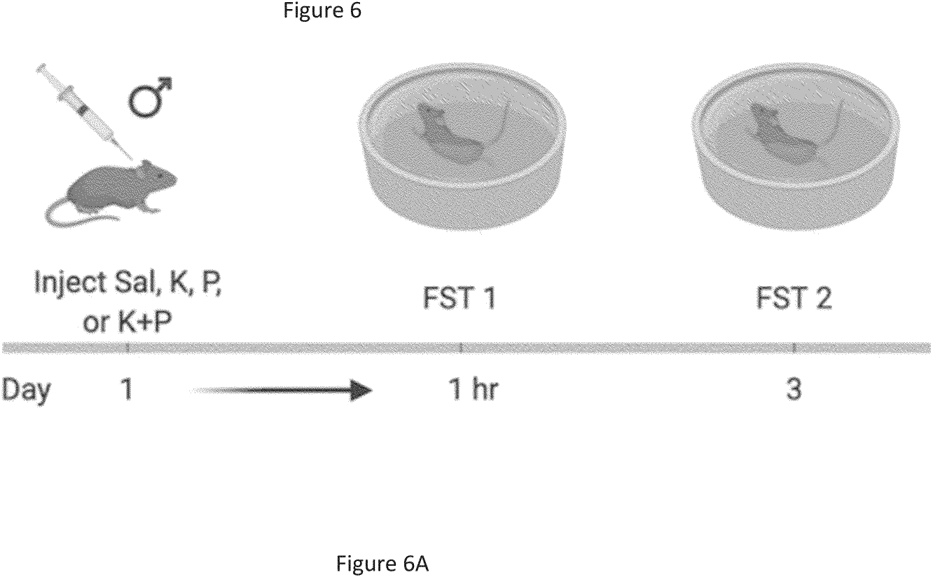

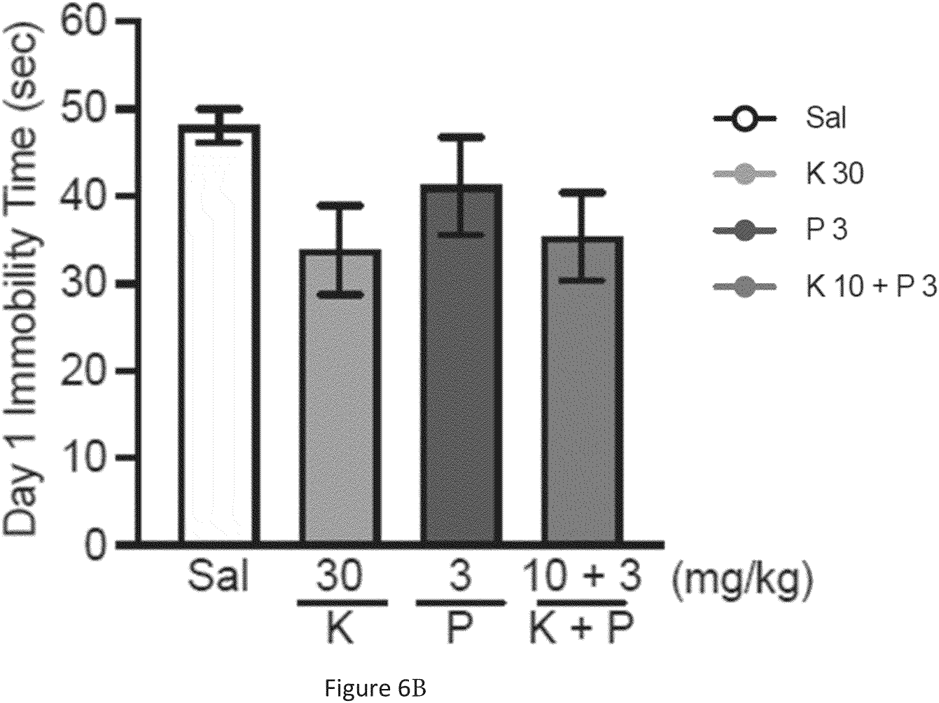

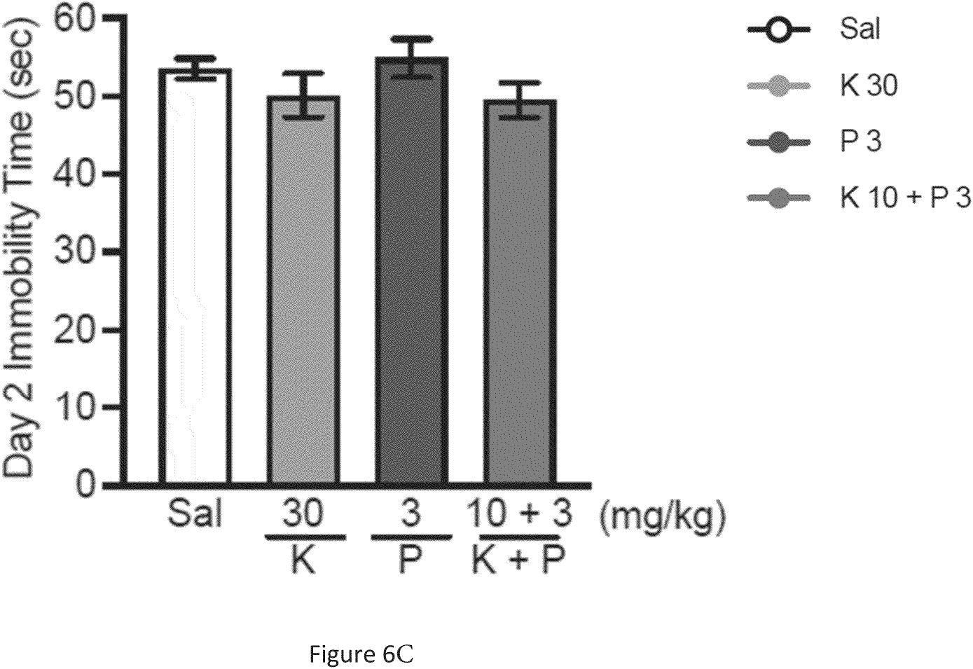

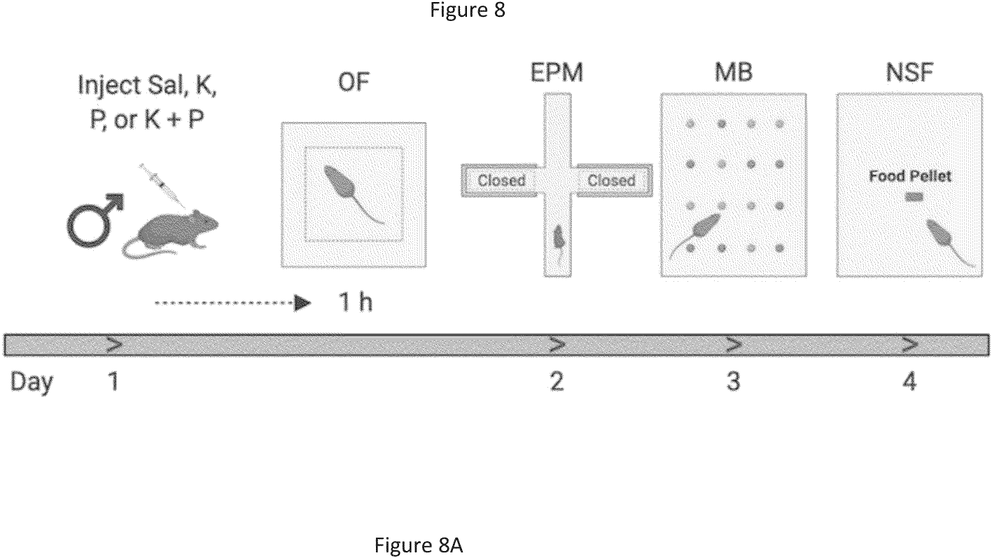

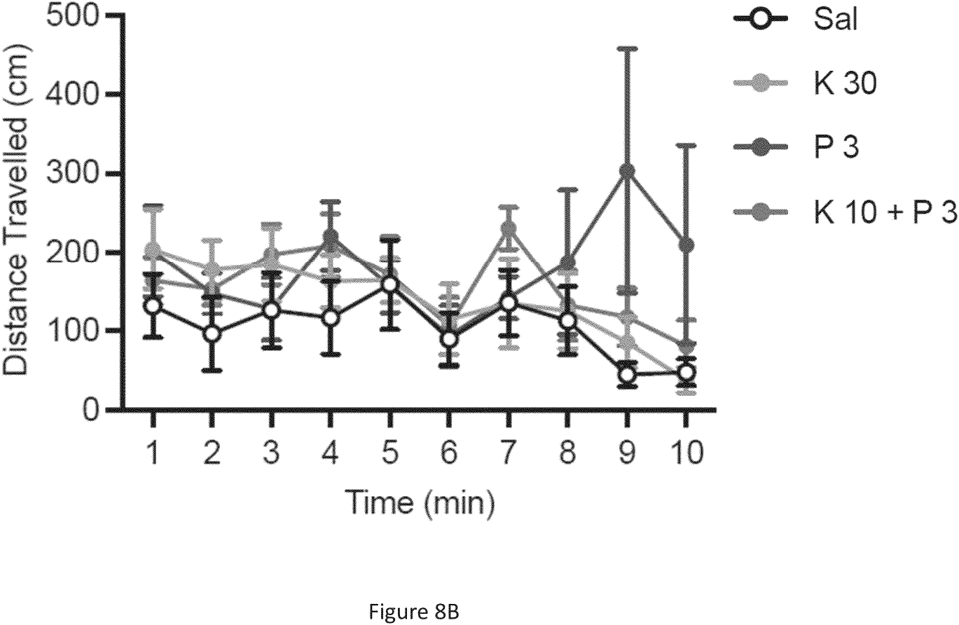

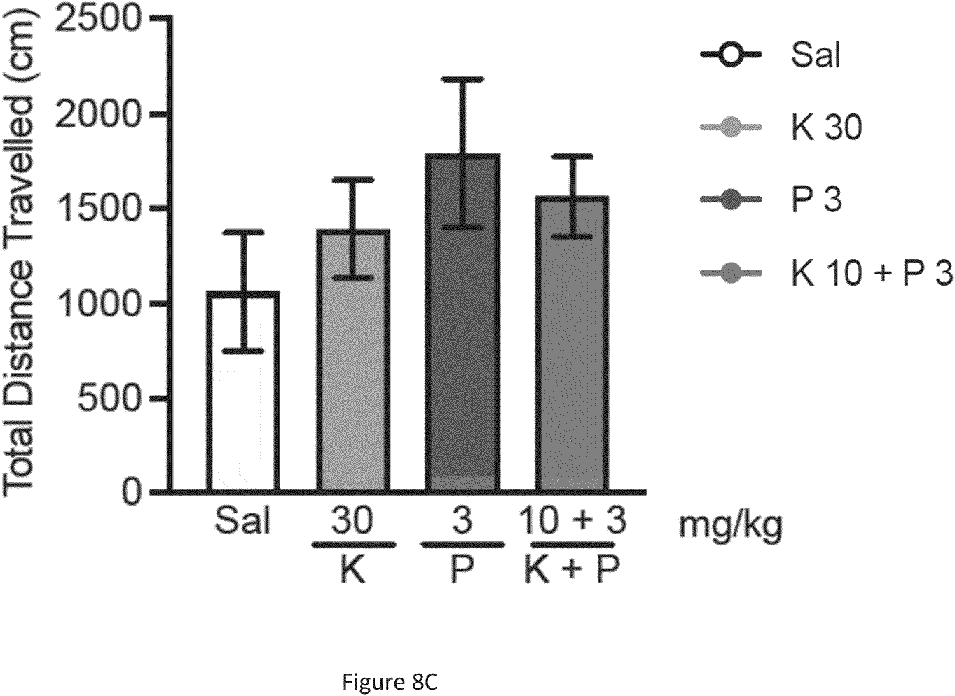

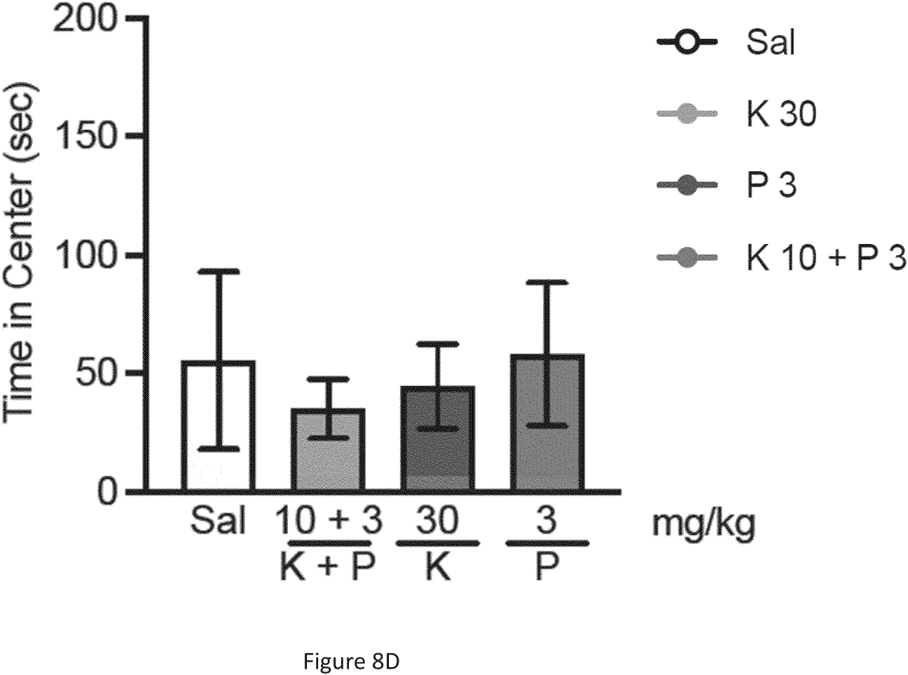

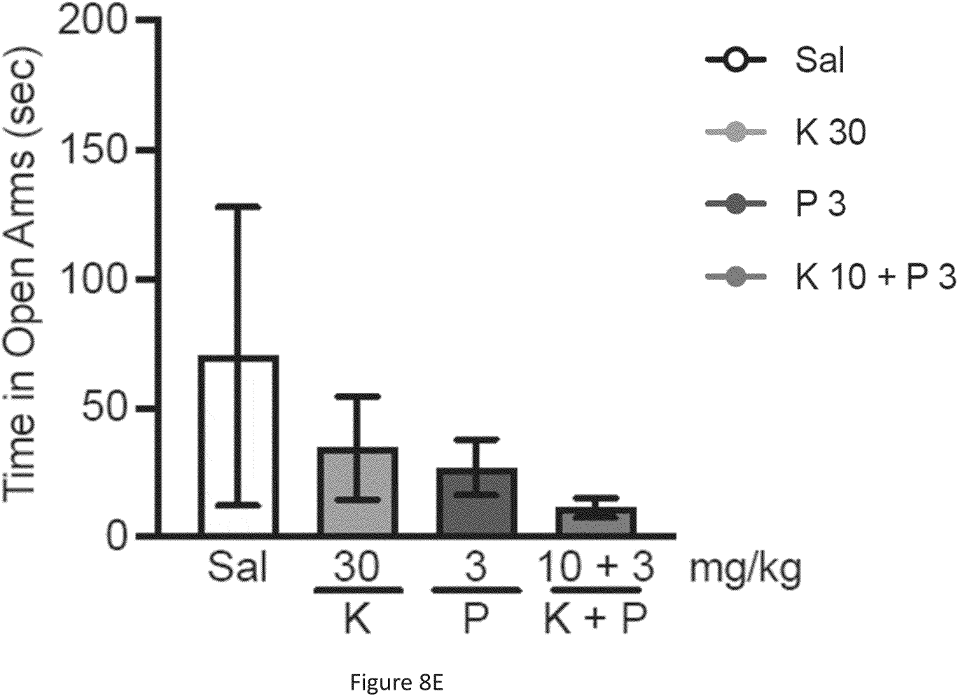

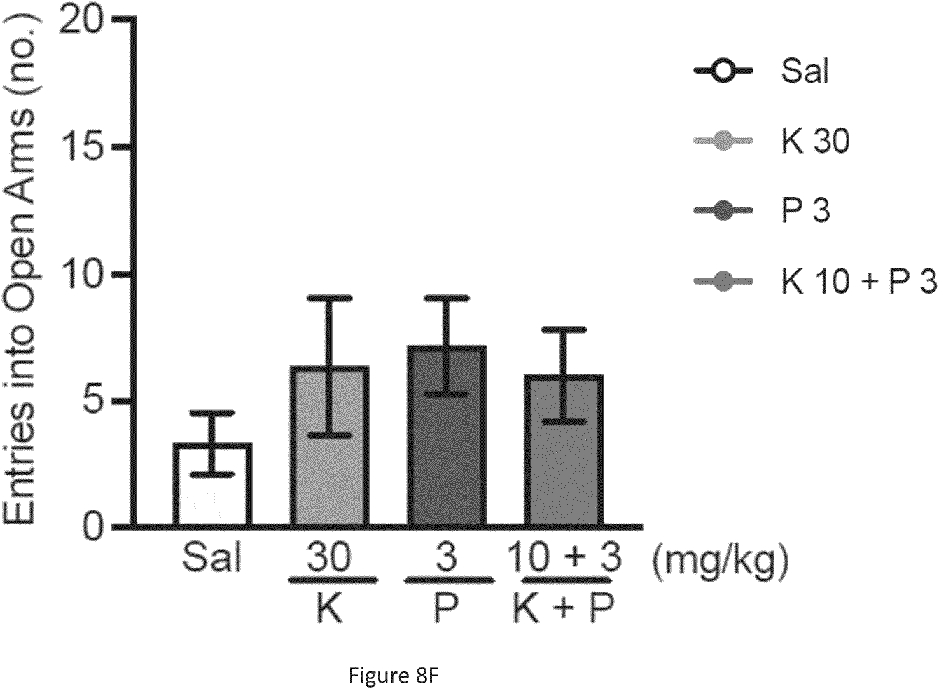

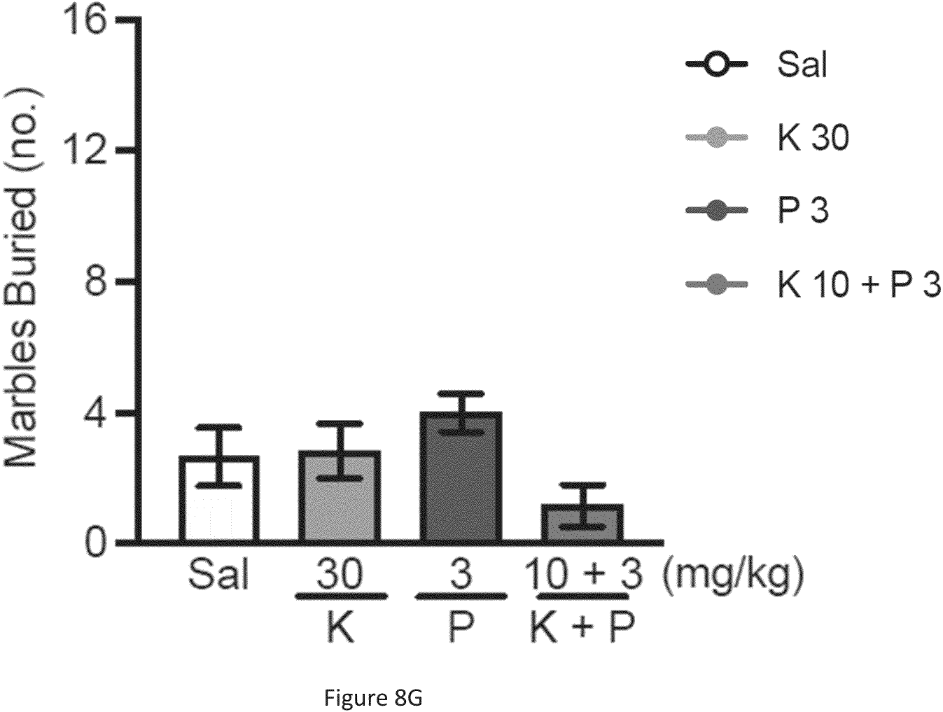

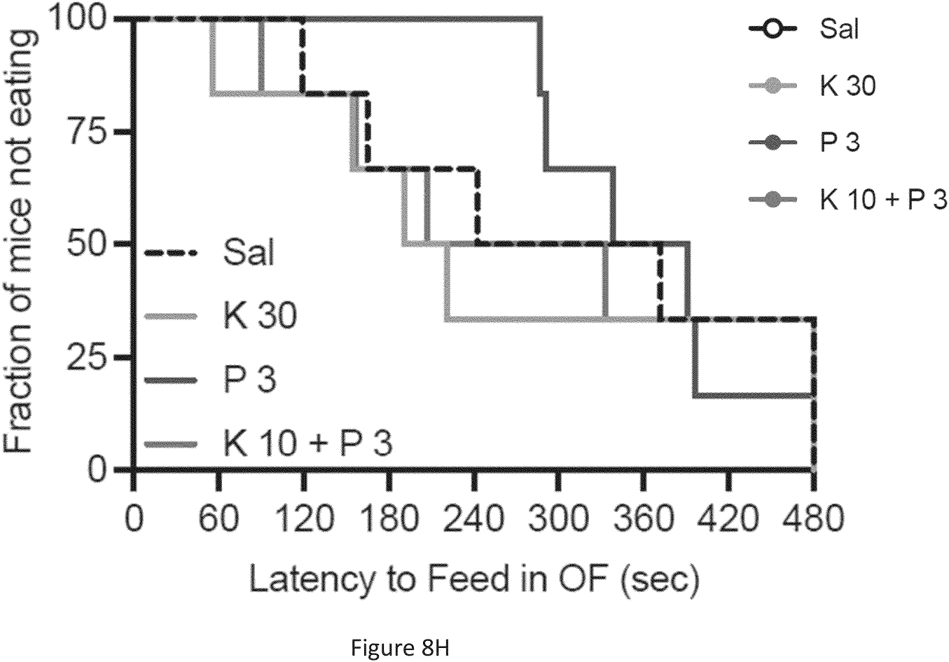

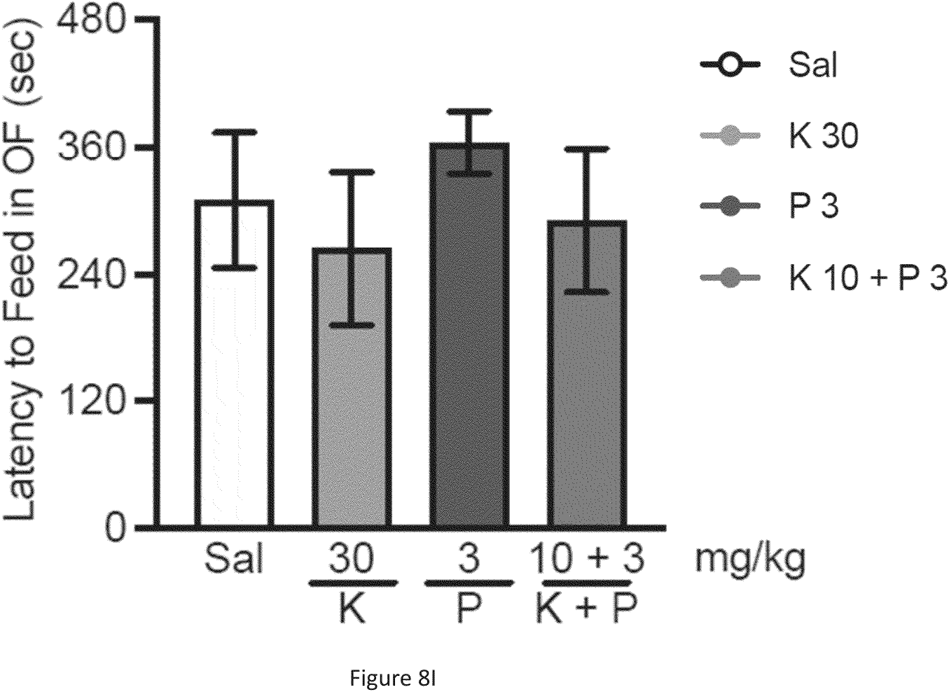

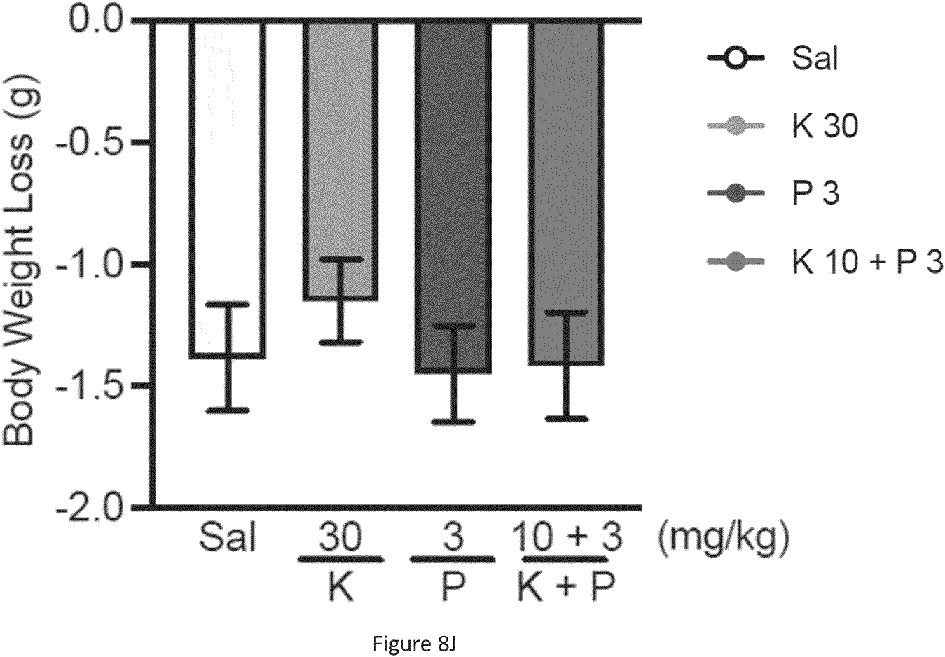

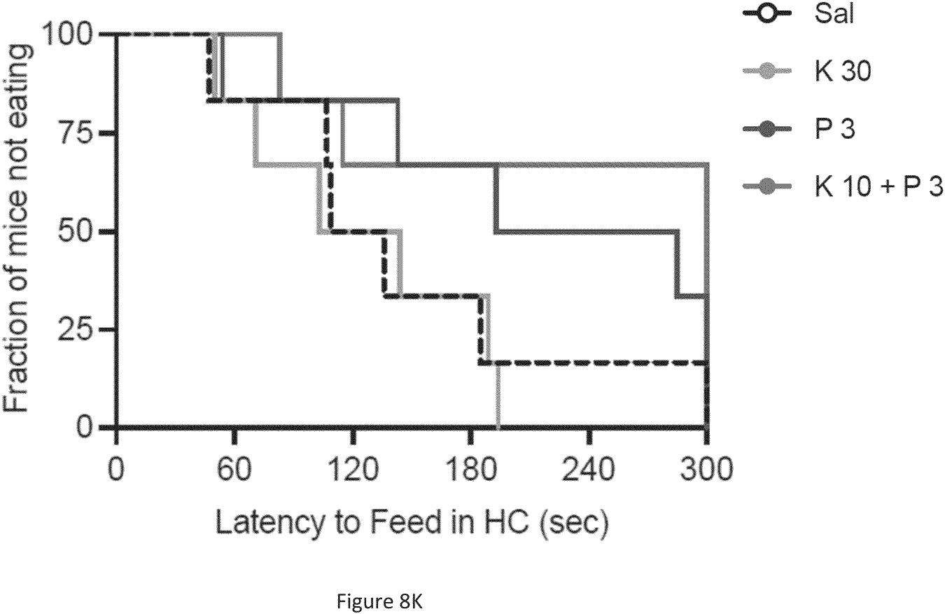

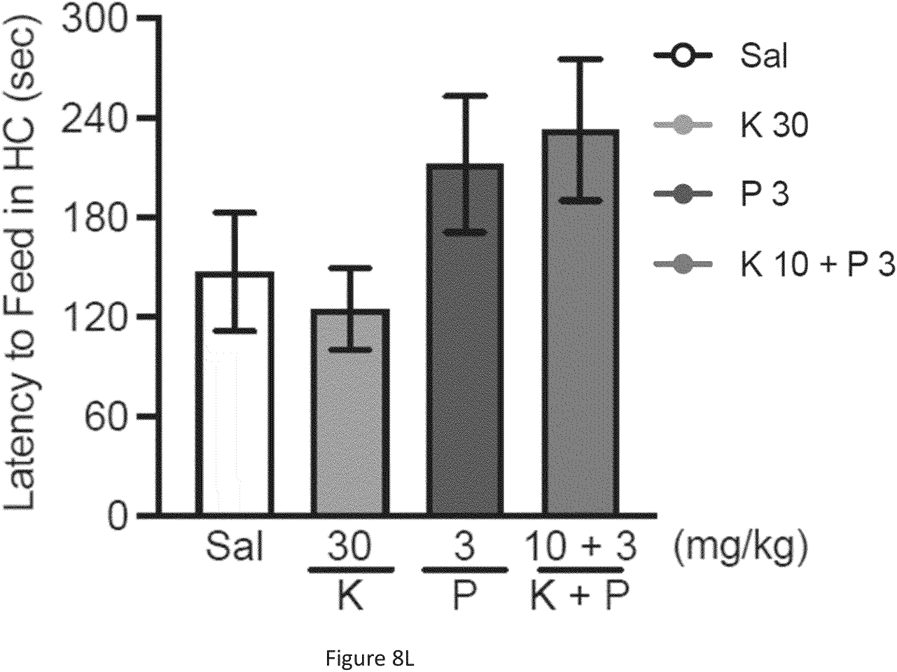

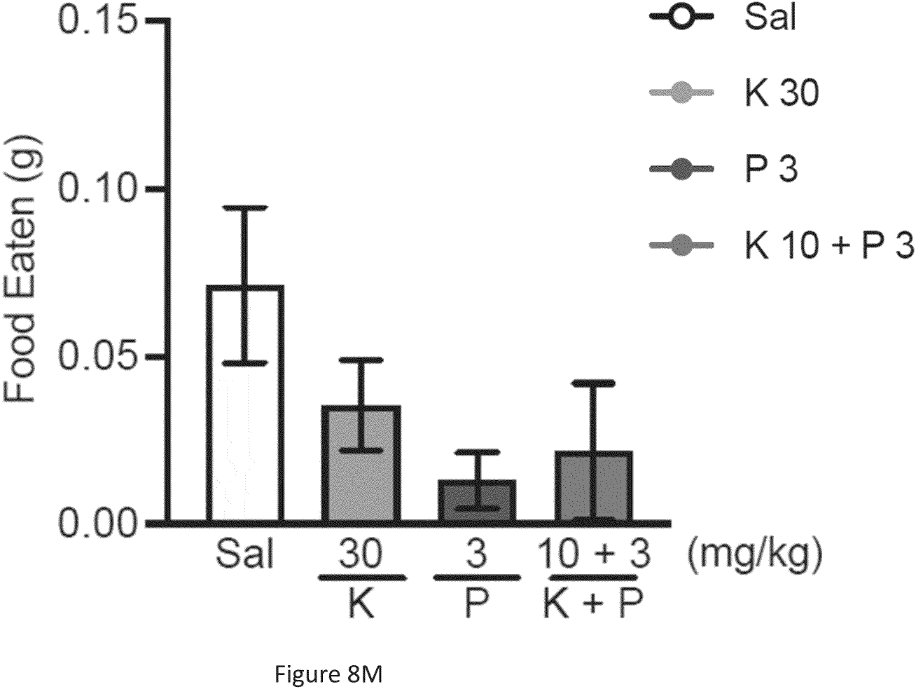

In these studies, male mice were injected with saline, (R,S)-ketamine, prucalopride, or combined (R,S)-ketamine and prucalopride one hour prior to the FST day 1 (FIG. 6A) or open field (OF). Behavior was also assessed using EPM, MB, and NSF (FIG. 8A). No stressor, e.g., 3-shock CFC paradigm, was administered.

Immobility times in the FST at day 1 and day 2 were comparable across all groups. See FIGS. 6B and 6C. Additionally, behavior was comparable across all groups in the OF (FIGS. 8B-8D) as well as in the EPM (FIGS. 8E and 8F), the MB (FIG. 8G), and the NSF (FIGS. 8H-8M).

### Example 7 - Combined (R,S)-Ketamine and Prucalopride Does not Alter Behavioral Despair but Reduces Perseverative Behavior in Non-Stressed Females

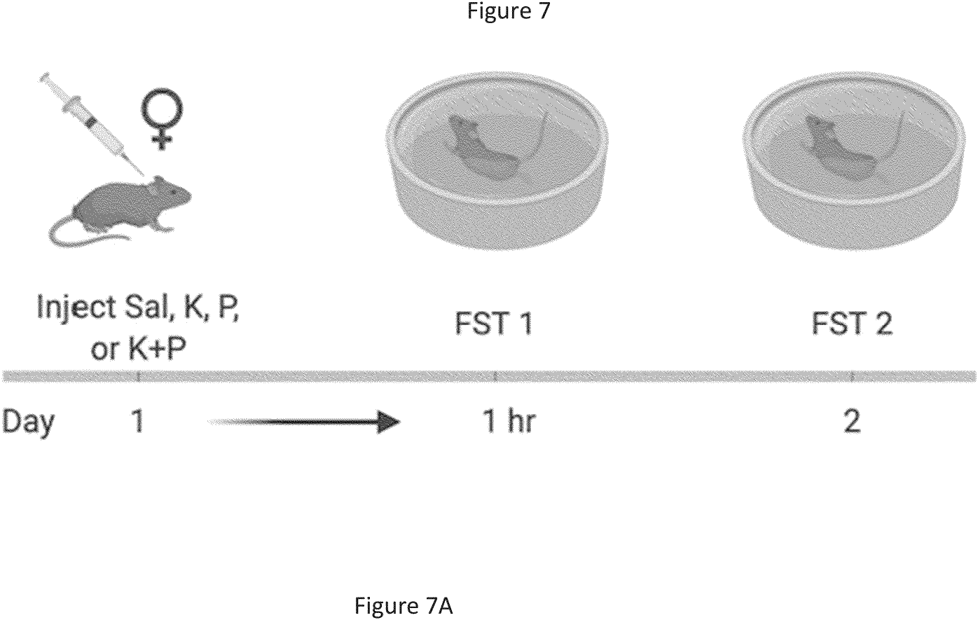

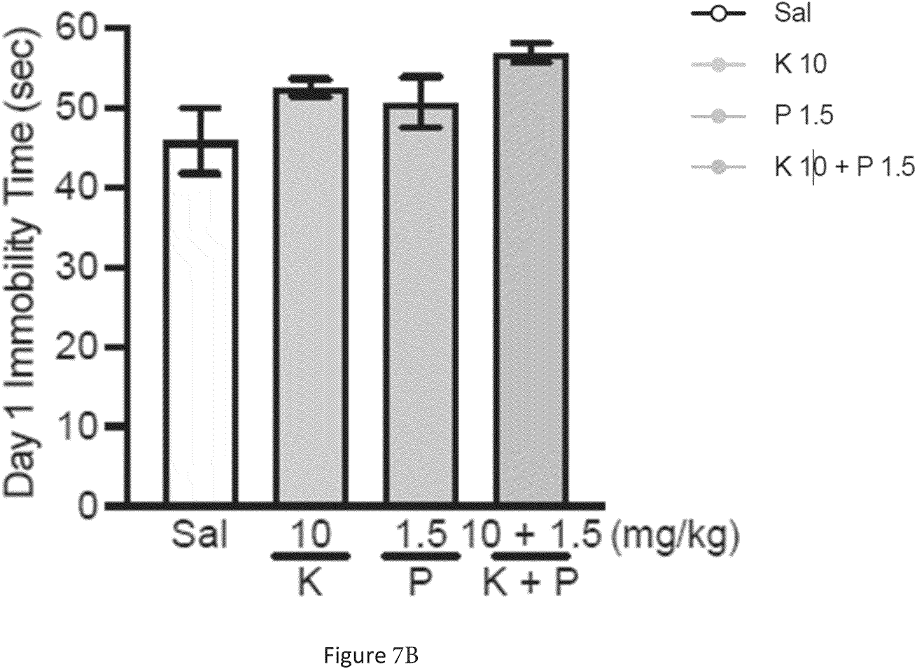

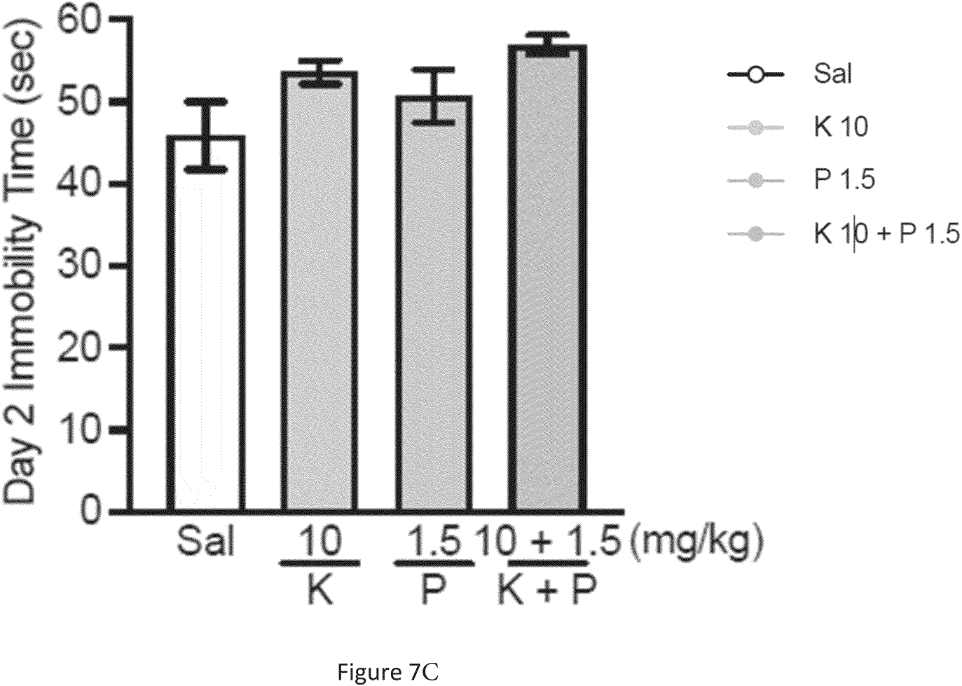

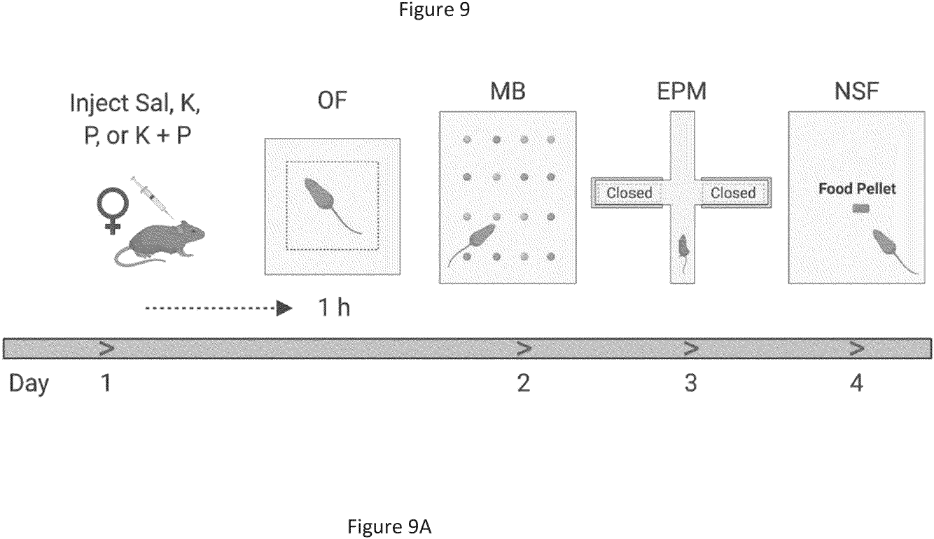

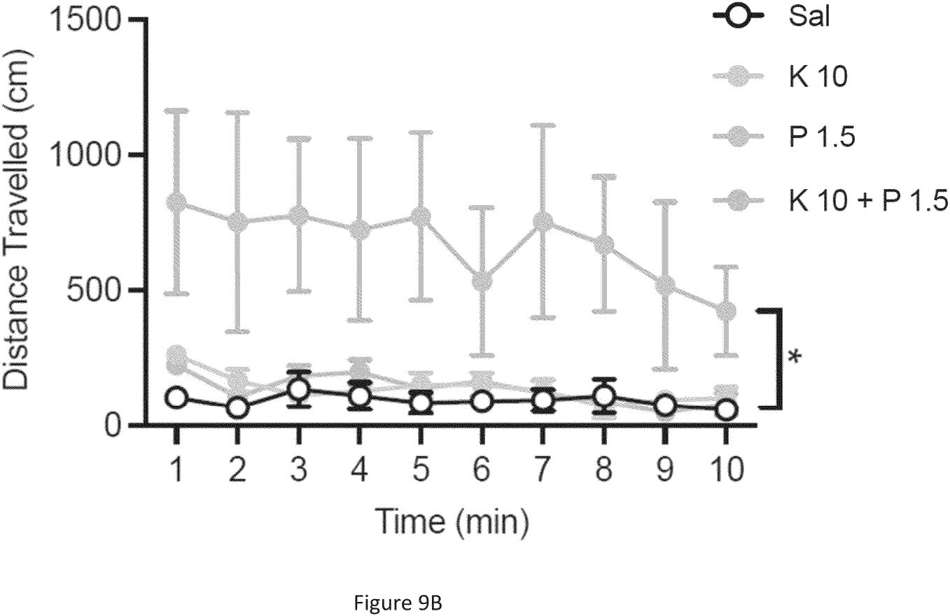

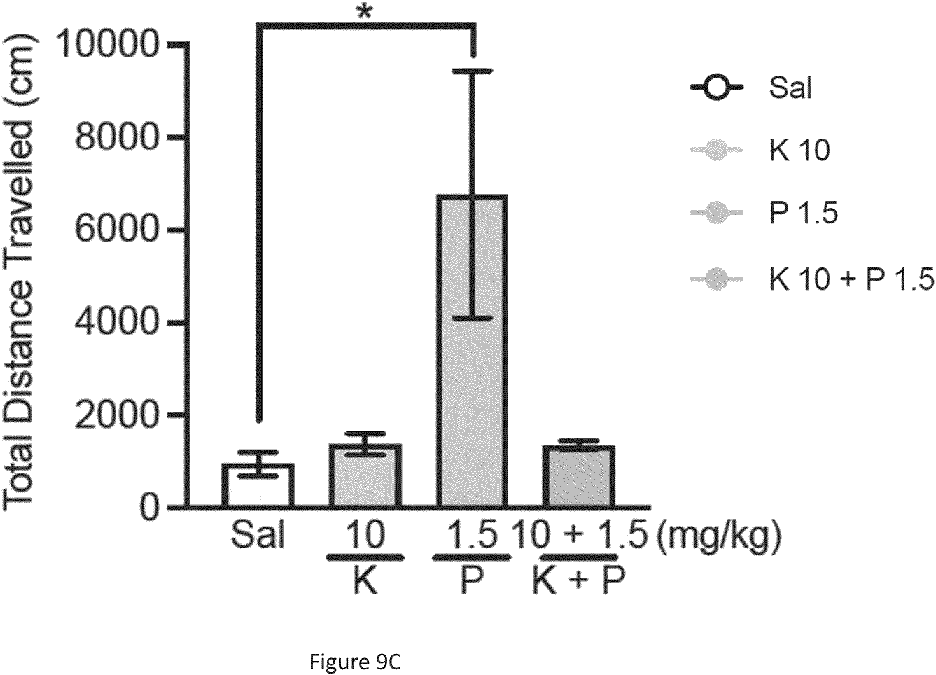

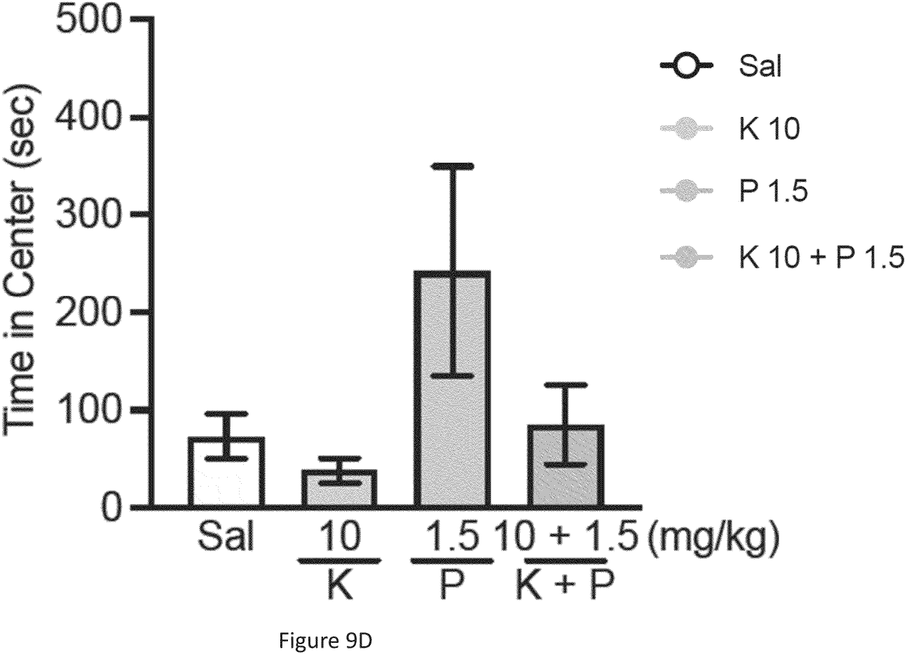

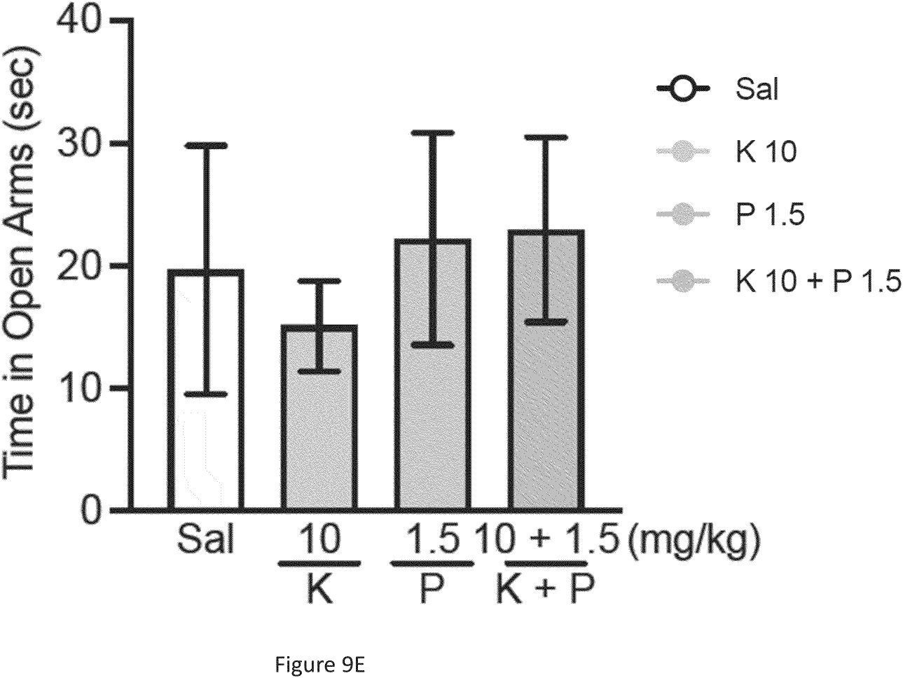

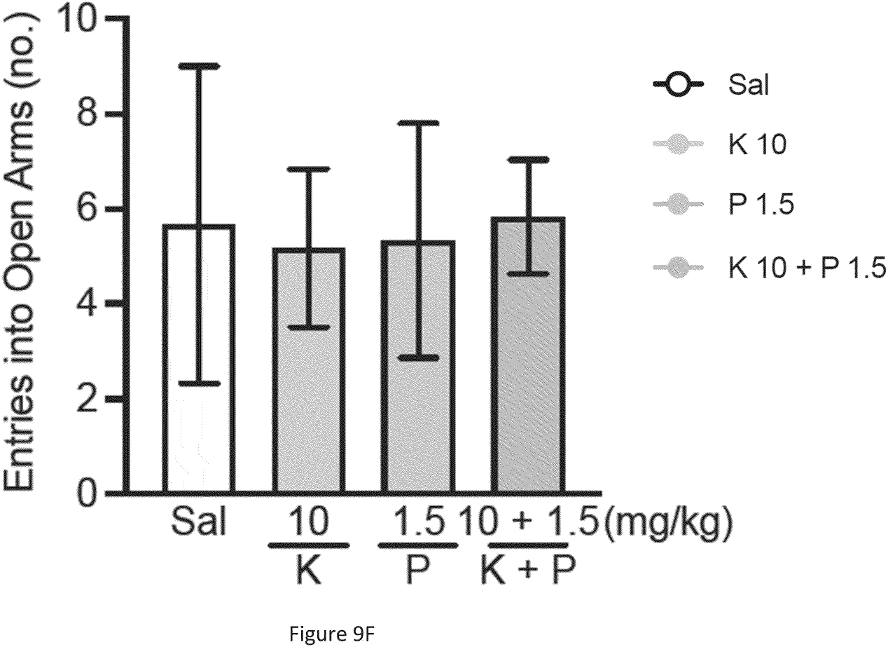

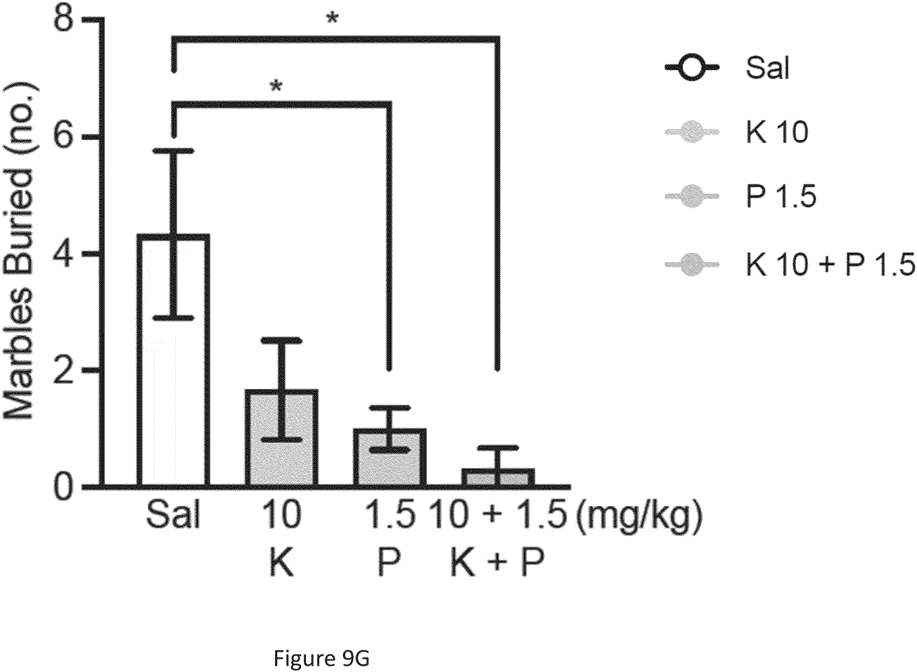

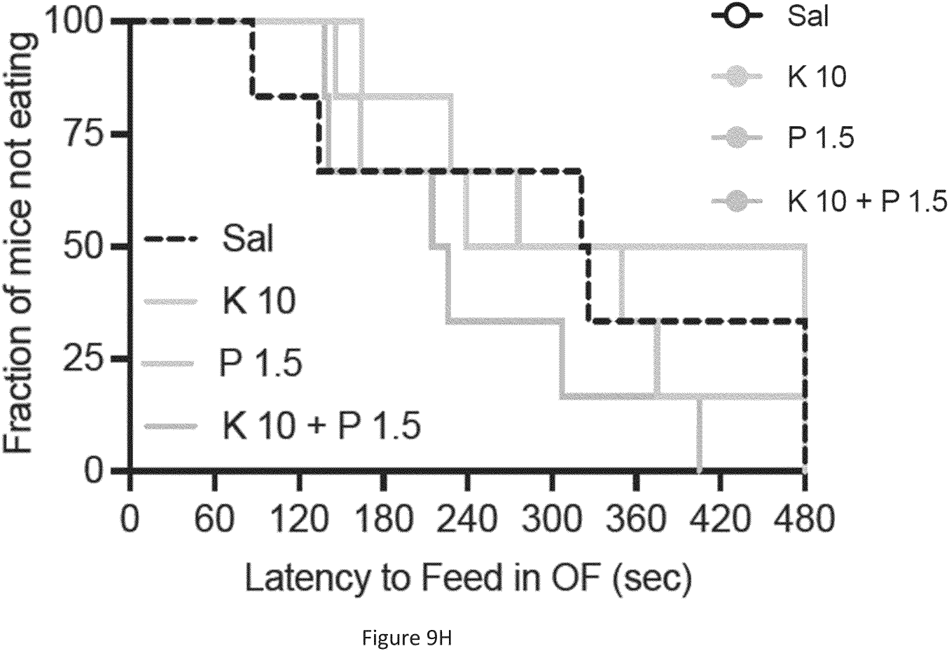

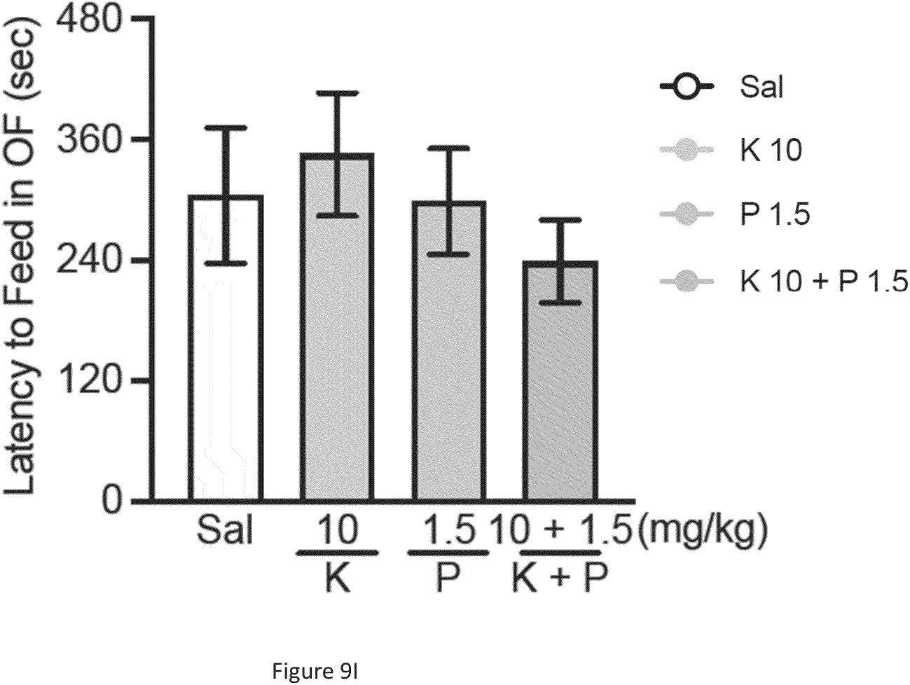

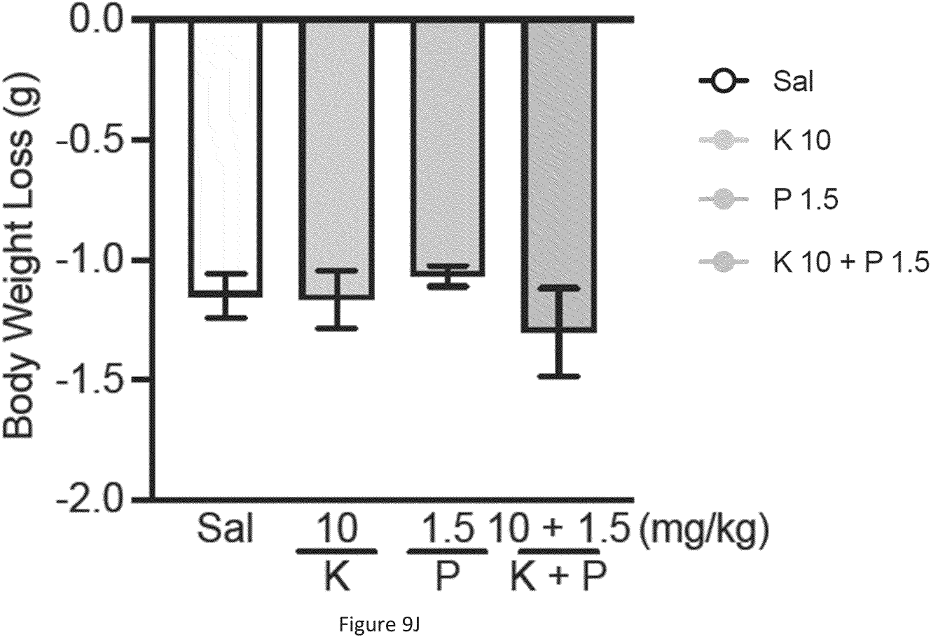

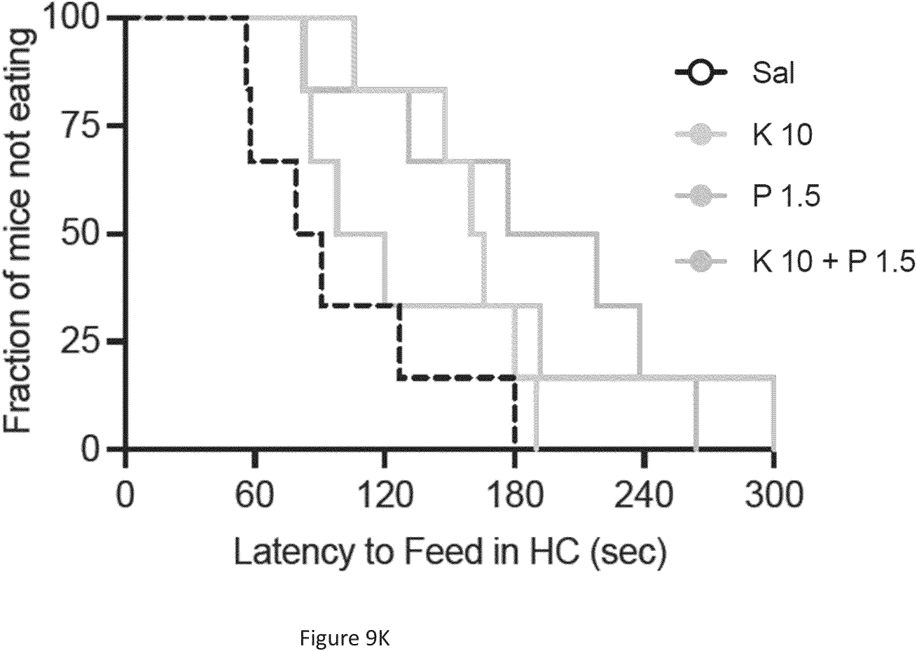

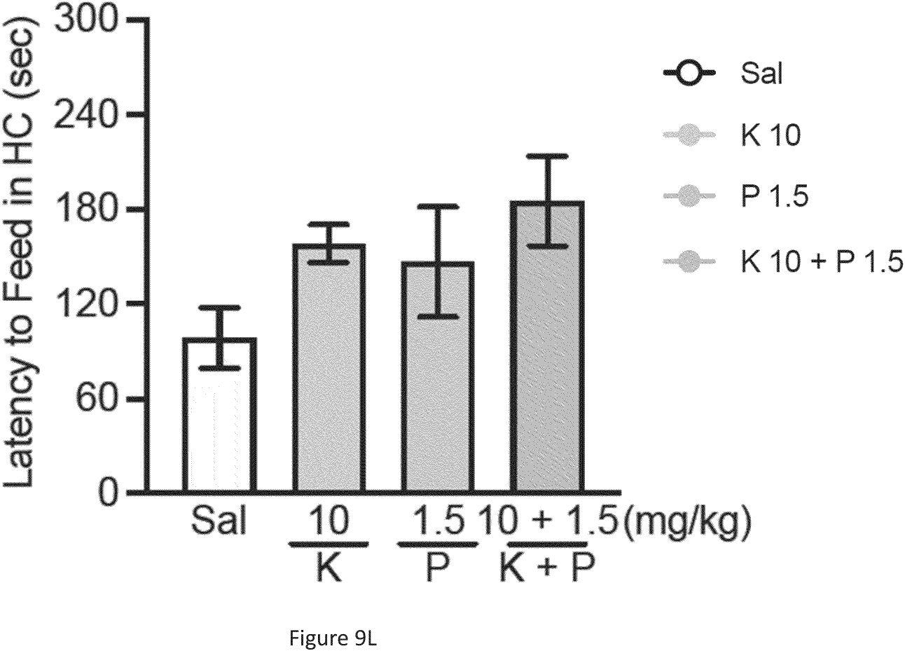

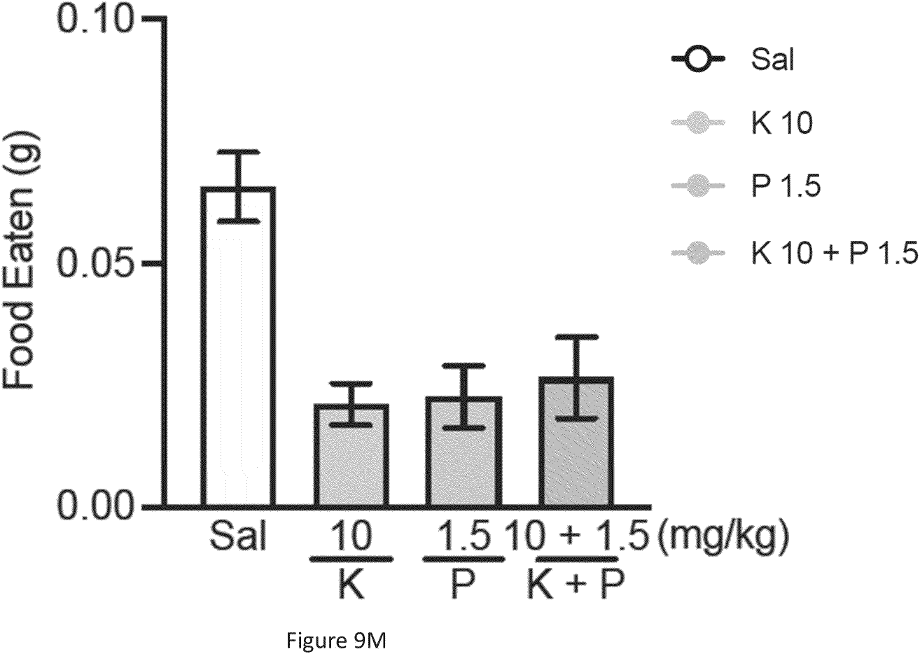

In these studies, female mice were injected with saline, (R,S)-ketamine, prucalopride, or combined (R,S)-ketamine and prucalopride one hour prior to the FST day 1 (FIG. 7A) or open field (OF). Behavior was also assessed using EPM, MB, and NSF (FIG. 9A). No stressor, e.g., 3-shock CFC paradigm, was administered.

Immobility times in the FST at day 1 and day 2 were comparable across all groups. See FIGS. 7B and 7C. Additionally, behavior was comparable across all groups in the OF (FIGS. 9B-9D) as well as in the EPM (FIGS. 9E and 9F) and the NSF (FIGS. 9H-9M).

Mice given prucalopride (1.5 mg/kg) or (R,S)-ketamine + prucalopride (10 + 1.5 mg/kg) significantly reduced the number of marbles buried in the MB assay (FIG. 9G).

These data indicated that the combined dosing of (R,S)-ketamine and prucalopride reduced perseverative behavior in female mice, even in the absence of exposure to stress.

### References

Amat et al. Previous ketamine produces an enduring blockade of neurochemical and behavioral effects of uncontrollable stress. J Neurosci. 2016;36:153-61.
Amigo et al. The absence of 5-HT₄ receptors modulates depression- and anxiety-like responses and influences the response of fluoxetine in olfactory bulbectomised mice: Adaptive changes in hippocampal neuroplasticity marker and 5-HT₁A autoreceptor. Neuropharmacology. 2016; 111:47-58.
Brachman et al. Ketamine as a Prophylactic Against Stress-Induced Depressive-like Behavior. Biological psychiatry 2016; 79(9):776-786.
Castello, Alterations of Expression of the Serotonin 5-HT₄ Receptor in Brain Disorders. Int J Mol Sci. 2018; 19.
Chen et al. Prophylactic efficacy of 5-HT₄R agonists against stress. Neuropsychopharmacology 2020; 45:542-52.
David et al. Neurogenesis-Dependent and -Independent Effects of Fluoxetine in an Animal Model of Anxiety/Depression. Neuron 2009; 62(4):479-493.
Drew et al. Arrest of adult hippocampal neurogenesis in mice impairs single- but not multiple-trial contextual fear conditioning. Behav Neurosci. 2010; 124(4):446-54.
Denny et al. Hippocampal Memory Traces Are Differentially Modulated by Experience, Time, and Adult Neurogenesis. Neuron. 2014; 83(1):189-201.
Dolzani et al. Inhibition of a descending prefrontal circuit prevents ketamine-induced stress resilience in females. eNeuro. 2018;5:pii: ENEURO.0025-18.2018.
Eglen et al. Pharmacological characterization of two novel and potent 5-HT₄ receptor agonists, RS 67333 and RS 67506, in vitro and in vivo. Br J Pharmacol. 1995; 115(8):1387-92.
Fava and Davidson. Definition and epidemiology of treatment-resistant depression. Psychiatr Clin North Am. 1996; 19(2):179-200.
Giannoni et al. Early administration of RS 67333, a specific 5-HT₄ receptor agonist, prevents amyloidogenesis and behavioral deficits in the 5XFAD mouse model of Alzheimer’s disease. Front Aging Neurosci. 2013; 5:96.
Grimwood et al. Translational receptor occupancy for the 5-HT₄ partial agonist PF-04995274 in rats, non-human primates and healthy volunteers. Alzheimer’s & Dementia: The Journal of the Alzheimer’s Association. 2011; 7(4):S653.
Mastrodonato et al. Ventral CA3 activation mediates prophylactic ketamine efficacy against stress-induced depressive-like behavior. Biol. Psychiatry 2018; 84:846-56.
Matthews et al. Ketamine for Treatment-Resistant Unipolar Depression. CNS Drugs. 2012; 1-16.
McGowan et al. Prophylactic ketamine attenuates learned fear. Neuropsychopharmacology. 2017; 42(8):1577-89.
Morris et al. Correction to “Synthesis and N-Methyl-d-aspartate (NMDA) Receptor Activity of Ketamine Metabolites”. Org Lett. 2017; 19(19):5494.
Morris et al. Synthesis and N-Methyl-d-aspartate (NMDA) Receptor Activity of Ketamine Metabolites. Org Lett. 2017; 19(17):4572-75.
Noppers et al., Absence of long-term analgesic effect from a short-term S-ketamine infusion on fibromyalgia pain: A randomized, prospective, double blind, active placebo-controlled trial. Eur. J. of Pain. 2011; 15(9):942-9.
Paskalis et al. Oral Administration of the NMDA Receptor Antagonist S-Ketamine as Add-on Therapy of Depression: A Case Series. Pharmacopsychiatry 2010; 40 :33-35.
Paul et al., Comparison of racemic ketamine and S-ketamine in treatment-resistant major depression: report of two cases. World J. of Bio. Psych. 2009; 10:241-244.
Prins et al. Pharmacological characterization of 5-HT₄ receptors mediating relaxation of canine isolated rectum circular smooth muscle. Br J Pharmacol. 1999; 127(6):1431-7.
Rebholz et al. Alterations of Expression of the Serotonin 5-HT₄ Receptor in Brain Disorders. Int J Mol Sci. 2018; 19.
Samuels et al. Serotonin 1A and Serotonin 4 Receptors: Essential Mediators of the Neurogenic and Behavioral Actions of Antidepressants. Neuroscientist. 2016; 22:26-45.
Saxe et al. Ablation of hippocampal neurogenesis impairs contextual fear conditioning and synaptic plasticity in the dentate gyrus. Proc Natl Acad Sci 2006; 103(46): 17501-17506. Vidal et al. Serotonin 5-HT₄ receptors: A new strategy for developing fast acting antidepressants? Curr Pharm Des. 2014; 20:3751-3762.
Yang et al. R-ketamine: a rapid-onset and sustained antidepressant without psychotomimetic side effects. Transl Psychiatry 2015; 5:e632.
Zanos and Gould. Intracellular Signaling Pathways Involved in (S)- and (R)-Ketamine Antidepressant Actions. Biol Psychiatry. 2018; 83(1):2-4.
Zanos et al. NMDAR inhibition-independent antidepressant actions of ketamine metabolites. Nature. 2016; 533(7604):481-6.

1. A method for preventing or delaying a stress-induced affective disorder or stress-induced psychopathology in a subject, comprising administering an effective amount of a one or more compositions comprising an agonist of serotonin 4 receptor (5-HT₄R), or a pharmaceutically acceptable salt, analog, derivative, or metabolite thereof, and ketamine, a ketamine analog, or a pharmaceutically acceptable salt, derivative, or metabolite thereof, wherein the administration of the one or more compositions prevents or diminishes three types of stress-induced maladaptive behaviors: fear; depressive-like; and anxiety-like behavior.

2. (canceled)

3. A method for preventing or delaying a stress-induced affective disorder or stress-induced psychopathology in a subject, comprising administering an effective amount of a one or more compositions comprising an agonist of serotonin 4 receptor (5-HT₄R), or a pharmaceutically acceptable salt, analog, derivative, or metabolite thereof, and an antagonist of the glutamate N-methyl-D-aspartate (NMDA) receptor (NMDAR), wherein the administration of the one or more compositions prevents or diminishes three types of stress-induced maladaptive behaviors: fear; depressive-like; and anxiety-like behavior.

4. (canceled)

5. A method for preventing or delaying a stress-induced affective disorder or stress-induced psychopathology in a subject, comprising administering an effective amount of a one or more compositions comprising an agonist of serotonin 4 receptor (5-HT₄R), or a pharmaceutically acceptable salt, analog, derivative, or metabolite thereof, and an agonist of the α-amino-3-hydroxy-5-methyl-4-isoxazolepropionic acid (AMPA) receptor (AMPAR), wherein the administration of the one or more compositions prevents or diminishes three types of stress-induced maladaptive behaviors: fear; depressive-like; and anxiety-like behavior.

6. (canceled)

7. The method of claim 1, wherein the agonist of 5-HT₄R comprises 1-(4-amino-5-chloro-2-methoxyphenyl)-3-[1(n-butyl)-4-piperidinyl]-1-propanone HCl (RS-67,333), 4-amino-5-chloro-2,3-dihydro-N-[1-3-methoxypropyl)-4-piperidinyl]-7-benzofuran carboxamide monohydrochloride (prucalopride), 4-[4-[4-Tetrahydrofuran-3-yloxy)-benzo[d]isoxazol-3-yloxymethyl]-piperidin-1-ylmethyl]-tetrahydropyran-4-ol (PF-04995274), or combinations thereof.

8-9. (canceled)

10. The method of claim 1, wherein the ketamine is (R,S)-ketamine.

11. The method of claim 1, wherein the one or more compositions is administered to the subject prior to a stressor.

12. The method of claim 11, wherein the one or more compositions is administered to the subject about 48 hours to about 3 weeks prior to a stressor.

13. The method of claim 11, wherein the one or more compositions is administered to the subject about 72 hours to about 2 weeks prior to a stressor.

14. The method of claim 11, wherein the one or more compositions is administered to the subject about 1 week prior to a stressor.

15. The method of claim 11, wherein the one or more compositions is administered to the subject once prior to a stressor.

16. The method of claim 1, wherein the one or more compositions is administered to the subject after a stressor.

17. The method of claim 16, wherein the one or more compositions is administered to the subject about 1 hour to about 1 day after a stressor.

18. The method of claim 16, wherein the one or more compositions is administered to the subject once after a stressor.

19. The method of claim 1, wherein the one or more compositions is administered at least once to the subject before a stressor and then after a stressor.

20. The method of claim 1, wherein the one or more compositions is administered orally, intravenously, intranasally, or via injection to the subject.

21. The method of claim 1, wherein the stress-induced affective disorder is selected from the group consisting of major depressive disorder and posttraumatic stress disorder (PTSD).

22. The method of claim 1, wherein the stress-induced affective disorder is selected from the group consisting of: stress-induced psychopathology; depressive-like behavior and associated affective disorders; anhedonic behavior and associated affective disorders; anxiety and associated affective disorders; cognitive impairments and deficits and associated disorders; stress-induced fear; and combinations thereof.

23. The method of claim 1, wherein the stress-induced affective disorder comprises stress-induced psychopathology.

24. The method of claim 1, wherein the subject is a human.
